(19) United States
(12) Patent Application Publication
(10) Pub. No.: US 2008/0255498 A1
(43) Pub. Date: Oct. 16, 2008

(54) Sensitizer Solutions, Systems, and Methods of Use

(76) Inventor: Philip R. Houle, Sunnyvale, CA (US)

Correspondence Address: PETERS VERNY , L.L.P., 425 SHERMAN AVENUE, SUITE 230, PALO ALTO, CA, 94306, US

(21) Appl. No.: 12/036,984

(22) Filed: Feb. 25, 2008

Related U.S. Application Data
(63) Continuation-in-part of application No. PCT/US2006/033458, filed on Aug. 25, 2006.
(60) Provisional application No. 60/711,990, filed on Aug. 25, 2005.
(60) Provisional application No. 60/904,454, filed on Feb. 28, 2007.
(60) Provisional application No. 60/904,623, filed on Mar. 1, 2007.

(51) Int. Cl. A61N 1/30; A61L 2/02; A61L 2/08

(52) U.S. Cl. 604/20; 422/186; 422/127; 422/22; 422/20

(57) ABSTRACT

A kit cleaning system and method is disclosed. One embodiment of the present invention has a photosensitizer solution supplied by a pressurized nozzle to the target site. Specific application of the solution is to an oral or skin surface target site. The photosensitizer solution is illuminated with sensitizing light creating reactive chemical species. Pressure and a solvent having an elevated concentration of oxygen or oxygen species improve the efficiency of the killing of pathogens. Methods of using the system within an oral cavity are also disclosed.

### RELATED APPLICATIONS

This application is a Continuation in Part of PCT International Application No. PCT/US2006/033458 filed on Aug. 25, 2006, which claims the benefit of U.S. Provisional Application No. 60/711,990 filed on Aug. 25, 2005; this application also claims the benefit of U.S. Provisional Application No. 60/904,454 filed on Feb. 28, 2007 and U.S. Provisional Application No. 60/904,623 filed on Mar. 1, 2007. All of the applications noted above are incorporated herein by reference in their entireties.

### BACKGROUND OF THE INVENTION

1. Field of the Invention

The invention relates generally to improved compositions that are photosensitizers and/or sonosensitizers and devices and methods for manufacture, application, and activation of those compositions to kill microorganisms and/or bleach colored compounds.

2. Description of the Related Art

Microorganisms are a major source of human, animal, and plant disease throughout the world and can infect virtually every part of the host organism. The primary treatment therapy is the administration of a chemical compound (e.g., antibiotics, biocides, fungicides or pesticides) that interferes with and/or prohibits a specific reaction or reaction type. This strategy of chemical inhibition tends to be narrowly focused on a single or small number of related chemical reactions. Due to this, small variations in the biochemistry of an organism or in its surrounding environment can prevent the chemical from being effective. For example, P. aeruginosa growing on urinary catheter material is 500-1000 times more resistant to antibiotics than the same cells growing in liquid culture (P. D. Marsh; Caries Research 2004; 38: 204-211) and exposure of microorganisms to insufficient levels of these chemical agents can lead to the selection of resistant strains. Based on these observations, it is clear that alternatives to this chemical inhibition strategy are needed for the direct prevention and treatment of disease as well as for the long-term control of microorganisms on non-biological surfaces.

Phototherapy is a term that includes all treatments that use light to induce reactions in the body that are of benefit to patients. Photodynamic therapy (PDT), a specific form of phototherapy, in general utilizes a photosensitizing compound (photosensitizer), which is a molecule having the ability to absorb light energy and use this energy to carry out chemical reactions. In addition to being used in PDT, photosensitizers have also been used for diagnostic purposes (photodiagnosis), such as fluorescent markers for example. Like PDT, photodiagnosis can also utilize photosensitizers that absorb a particular wavelength of light. In the case of fluorescence and phosphorescence photodiagnosis, the absorbed photon from the illuminating radiation excites a photosensitizer's electron from a ground state to a higher state. The excited electron then falls to a lower level, but not immediately back to the ground state, emitting a longer-wavelength photon than it absorbed thereby allowing a practitioner to easily differentiate the labeled structures, for example cells, from the normal tissue by using a fluorescence scanner, for example. Sonodynamic therapy (SDT) in general utilizes a sonosensitizing compound (sonosensitizer), which is a molecule having the ability to absorb vibration energy, for example in the form of ultrasound or sonoluminescence, and use this energy to carry out chemical reactions.

When energy from an appropriate source is applied, photosensitizers and sonosensitizers (hereafter referred to alternately as sensitizers) produce a toxic effect through the production of reactive chemical species (RCS), for example reactive oxygen species (ROS). In particular, it is believed that singlet oxygen, an ROS, is responsible for much of the toxicity of these sensitizers. RCS, for example hydrogen peroxide, are also well known for their ability to kill bacteria and pathogens, and to bleach colored compounds.

Sensitizers, in combination with an appropriate energy source, have shown some level of effectiveness in killing a broad range of microorganisms in planktonic suspension including both drug sensitive and drug resistant varieties. They have also shown promise in killing and/or limiting the growth of organisms in homogeneous and/or mixed species biofilms. Their use for bleaching hard tooth structures is well known, and several products for both professional and home-based application are commercially available. Their successful application, however, faces significant challenges and can depend on several factors related to the sensitizer including; chemical toxicity, pharmacokinetics, stability, delivery, distribution, and specificity. Successful application can also depend heavily on the type and efficiency of the RCS produced, penetration of the activating energy, and availability and performance of the energy source.

For many applications it is advantageous for the sensitizer to be non-toxic when not exposed to an appropriate energy source, and stable during storage and use. As well, RCS are highly reactive surviving for only a short time, many on the order of microseconds, before being involved in a quenching reaction. Because of this it is advantageous for the sensitizer, in combination with its delivery and activation devices and methods, to enable the production of high quantities of RCS inside or in the immediate vicinity of the targeted organism or colored compound.

Use of photosensitizer and sonosensitizer compositions, and the associated devices and methods, has been limited in the ability of these art approaches to produce sufficient quantities of RCS in the required locations to achieve the desired level of bleaching or toxicity in the targeted compounds, organisms and/or cells.

H. Alimi in US Patent Publications 2005/1039808 A1 published Jun. 30, 2005 (as U.S. Ser. No. 10/862,092, filed Jun. 4, 2004), 2005/0142157 A1, published Jun. 30, 2005 (as U.S. Ser. No. 10/916,566, filed Aug. 11, 2004) and 2005/0196462 A1 published Sep. 8, 2005 (as U.S. Ser. No. 10/916,278, filed Aug. 11, 2004) describe oxidative reduction potential water solutions that are stable for hours, equipment and methods for the manufacture of these solutions, and a corresponding method that the compositions are useful to treat a condition in a patient, e.g., wound healing and anti-bacterial action.

The TherOx Corporation of Irvine Calif. has reported solutions, methods, emulsions and equipment to increase oxygen content in compositions, e.g., pharmaceuticals. Some patent and patent publications of interest are found in the following Tables 1 and 2, all of which are incorporated by reference.

TABLE 1
U.S. Patents of TherOx

| Patent No. | Title |
|---|---|
| 6,461,590 | Stabilized Gas-Supersaturated Suspensions and Methods for their Delivery |
| 6,344,489 | Stabilized Gas-Enriched and Gas-Supersaturated Liquids |
| 6,315,942 | System for Delivery of Gas-Enriched Fluids |
| 6,312,647 | Method for Enriching a Fluid with Oxygen |
| RE 37,379 | High Pressure Gas Exchanger |
| 6,241,802 | Method for Delivery of Gas-Enriched Fluids |
| 6,238,645 | Methods of Oxygenating Tissues |
| 6,197,279 | Methods of Delivering Oxygen with Emulsions |
| 6,169,117 | Stabilized Gas-Supersaturated Emulsions |
| 5,958,377 | Methods of Delivering Gases to Gas Depleted Environments |
| 5,922,305 | Methods of Treating Tissues with Oxygen-Supersaturated Emulsions |
| 5,834,519 | Stabilized Gas-Supersaturated Emulsions and Suspensions |
| 5,797,874 | Method of Delivery of Gas-Supersaturated Liquids |
| 5,735,934 | Method for Delivering a Gas-Supersaturated Fluid to a Gas-Depleted Site and Use Thereof |
| 5,693,017 | Apparatus and Method of Delivery of Gas-Supersaturated Solutions to a Delivery Site |
| 5,599,296 | Apparatus and Method of Delivery of Gas-Supersaturated Liquids |
| 5,569,180 | Method for Delivering a Gas-Supersaturated Fluid to a Gas-Depleted Site and Use Thereof |
| 5,407,426 | Method and Apparatus for Delivering Oxygen into Blood |
| 5,261,875 | Method and Apparatus for Injection of Gas Hydrates |
| 5,086,620 | Method of Microencapsulation of Hyperbaric Gas |
| 5,084,011 | Method for Oxygen Therapy Using Hyperbarically Oxygenated Liquid |
| 6,782,924 | System and Method for High Pressure Delivery of Gas-Supersaturated Fluids |
| 6,759,008 | Apparatus and Method for Blood Oxygenation |
| 6,746,417 | Apparatus for Generalized Extracorporeal Support |
| 6,676,900 | Method for the Preparation and Delivery of Gas-Enriched Fluids |
| 6,622,542 | Bubble Detector and Method of Use Thereof |
| 6,613,280 | Disposable Cartridge for Producing Gas-Enriched Fluids |
| 6,607,698 | Method for Generalized Extracorporeal Support |
| 6,605,217 | Apparatus for High Pressure Fluid Filtration |
| 6,602,468 | Method of Blood Oxygenation |
| 6,602,467 | Apparatus and Method for Blood Oxygenation |
| 6,596,235 | Method for Blood Oxygenation |
| 6,582,387 | System for Enriching a Bodily Fluid with a Gas |
| 6,576,191 | Apparatus for Blood Oxygenation |
| 6,565,807 | Method of Blood Oxygenation |
| 6,558,502 | Method for forming Atraumatic Fluid Delivery Device |
| 6,555,059 | Method of Forming Gas-Enriched Fluid |
| 6,533,766 | Coating Medical Device Surfaces for Delivering Gas-Supersaturated Fluids |
| 6,454,997 | Apparatus for the Preparation and Delivery of Gas-Enriched Fluids |
| 6,387,324 | Apparatus and Method fro Blood Oxygenation |
| 6,346,192 | Apparatus for High Pressure Fluid |
| 6,315,754 | System and Method for High Pressure Delivery of Gas-Supersaturated Fluids |
| 6,248,087 | Apparatus for Generalized Extracorporeal Support |
| 6,235,007 | Atraumatic Fluid Delivery Devices |
| 6,180,059 | Method for the Preparation and Delivery of Gas-Enriched Fluids |
| 6,142,971 | System and Method for High Pressure Delivery of Gas-Supersaturated Fluids |
| 6,123,698 | Angioscopy Apparatus and Methods |
| 6,030,357 | System and Method for High Pressure Delivery of Gas-Supersaturated Fluids |
| 5,976,119 | High Pressure Perfusion Device |
| 5,957,899 | High Pressure Transluminal Fluid Delivery Device |
| 5,893,838 | System and Method for High Pressure Delivery of Gas-Supersaturated Fluids |
| 5,797,876 | High Pressure Perfusion Device |

TABLE 2
U.S. Patent Publications of TherOx

| | |
|---|---|
| 20060264811 | System and method for high pressure delivery of gas-supersaturated fluids”[method of |
| | making perfluorocarbon emulsions with non-fluorinated surfactants] |
| 20060193878 | Method of Preparing Gas Delivering Perfluorocarbon Emulsions With Non-Fluorinated |
| | Surfactants |
| 20060193799 | Method of Making Perfluorocarbon Emulsions With Non-Fluorinated Surfactants |
| 20060147492 | Medical implants and anti-scarring agents |
| 20060100639 | System and method for the treatment of reperfusion injury |
| 20060054554 | Method for oxygenating wastewater |
| 20050042132 | Apparatus for blood oxygenation |
| 20050028883 | System and method for high pressure delivery of gas-supersaturated fluids |
| 20050004503 | Method and apparatus for treating acute myocardial infarction with hypothermic perfusion |
| 20040166064 | System for storing and dispensing a gas-solubilized product |
| 20040154374 | System and method of evaluating or calibrating a bubble detector |
| 20040089050 | Bubble detector and method of evaluation or calibration thereof |
| 20040057906 | Perfluorocarbon emulsions with non-fluorinated surfactants |
| 20040019319 | Method for enriching a bodily fluid with a gas |
| 20040013566 | Disposable cartridge for producing gas-enriched fluids |
| 20030231982 | Method of forming gas-enriched fluid |
| 20030215356 | Method of blood oxygenation |
| 20030194348 | Apparatus for blood oxygenation |
| 20030161759 | Apparatus for blood oxygenation |
| 20030095892 | Apparatus for blood oxygenation |
| 20030091470 | Method of Blood Oxygenation |
| 20020192111 | Apparatus for the preparation and delivery of gas-enriched fluids |
| 20020161351 | Method and apparatus for treating acute myocardial infarction with selective hypothermic |
| | perfusion |
| 20020138034 | System for enriching a bodily fluid with a gas |
| 20020136662 | Disposable cartridge for producing gas-enriched fluids |
| 20020134134 | Bubble detector and method of evaluation or calibration thereof |
| 20020070182 | Apparatus for high pressure fluid filtration |
| 20020040232 | Method for forming atraumatic fluid delivery device |
| 20020032402 | System and method for high pressure delivery of gas-supersaturated fluids |
| 20010037975 | Apparatus for high pressure fluid filtration |
| 20010028893 | Stabilized gas-supersaturated suspensions and methods for their delivery |
| 20010016729 | Apparatus and method for blood oxygenation |
| 20010008960 | System and method for high pressure delivery of gas-supersaturated floids |

E. M. Oskar, Great Britain Patent 731 553 A (published 8 Jun. 1995).
TGA Tech Geraete Und Apparateb, German Patent 43 38 622 A1 (published 18 May 1995).
W. Neuberger, U.S. Pat. No. 6,056,548A (issued 2 May 2000).
A. Lanfranchi, US Patent Publication 2002/028420 A1 published 7 Mar. 2002).
P. Lustig, PCT International Application, WO 92/18063 A (published 29 Oct. 1992).

All articles, references, theses, books, standards, patents U.S. and foreign, U.S. patent applications and/or U.S. patent publications cited herein are incorporated herein by reference in their entirety.

It is apparent from the art that a need still exists for compositions, devices, and methods that increase sensitizer delivery, activation, and RCS production effectiveness in the vicinity of targeted compounds, cells, and/or organisms.

### BRIEF SUMMARY OF THE INVENTION

Embodiments of the inventions disclosed herein comprise oxygen, and/or other ROS, at partial pressures above ambient levels and the use of these embodiments to treat various medical conditions is described. For example, in some embodiments of the inventions described herein, the use of hyperbaric pressures and/or increased partial pressures are important to increase the concentration level of active oxygen species, thus, accelerating the destruction of the targeted tissues, and/or pathogens, thereby sterilizing and improving any infection, and/or precancerous, and/or cancerous condition and decreasing the time of healing.

A cleaning system for biological surfaces or structures having a fluid delivery system, a sensitizer composition, and a transducer, such as an illuminating device is described herein. Cleaning includes killing microorganisms, killing cells, treating disorders, coloring (including removing color from) tissue, treating a disease and/or symptoms of a disorder and/or disease, causing or increasing the rate of healing of a wound. The sensitizer composition is in a source in the fluid delivery system. In certain embodiments the source in the fluid delivery system is pressurized or non-pressurized. In certain embodiments the source is a container, can, ampoule, cartridge, syringe (e.g., a plunger-type mechanism), bag, tube, reservoir, squeeze bulb, external source, or combinations thereof.

In certain embodiments the pressure, energy source, solvent, chemical components and combinations and conditions thereof provide for an increase in the concentration of active reactive chemical species to act more quickly and more thoroughly. In certain embodiments the increase in the concentration of chemical species allows the use of a less intense energy source.

A transducer, such as an illumination device, having an electromagnetic energy transducer is disclosed herein. In certain embodiments the electromagnetic energy transducer has an illuminator. In certain embodiments the illuminator has a light emitting diode (LED).

An illumination device having an electromagnetic energy transducer and/or electric (e.g., to activate an electro-sensitizer, and/or for iontophoresis, electrophoresis) and/or magnetic field (e.g., for activating a magnetosensitizer and/or for transporting iron, oxygen) source is disclosed herein. In certain embodiments the electromagnetic energy transducer includes an illuminator. In certain embodiments the illuminator is an LED. In certain embodiments the electric field source has surfaces (e.g., electrodes) that are at a higher and/or lower electric potential than the structures and or regions around them thereby creating an electric potential gradient in which any charged particle experiences a force. In certain embodiments the magnetic field source includes a permanent magnet and/or electromagnet. In certain embodiments the electromagnet includes a loop of conductive material that creates a magnetic field when current is passed through the conductive material, such that any magnetic material (e.g., ferromagnetic, paramagnetic, superparamagnetic and/or diamagnetic) experiences a force.

An applicator, such as a mouthpiece (e.g., dental tray, bite block, flexible sheet) having an electromagnetic energy transducer is disclosed herein. In certain embodiments the electromagnetic energy transducer includes an illuminator. In certain embodiments the illuminator includes an LED.

Compositions containing one or more sensitizers and one or more perfluorocarbons and/or perfluorocarbon derivatives and/or perfluorocarbon precursors are disclosed herein. In certain embodiments one or more components of the compositions target the sensitizer to a specific target (e.g., microorganism, cell type, and the like). In certain embodiments one or more components of the compositions act as surfactants, thus lowering surface tension.

Embodiments that include sensitizer compositions containing gases at, and above, concentrations found under standard atmospheric conditions are disclosed herein. In certain embodiments the gas is oxygen, ozone, air, nitrogen, carbon dioxide, an inert gas (e.g., a noble gas) and/or mixtures thereof. In certain embodiments the compositions contain gas solubility-increasing compounds. In certain embodiments the gas solubility-increasing compounds include perfluorocarbons, and/or their derivatives, and/or their precursors, and/or hemoglobin, and/or modified hemoglobin compounds, and/or their precursors. In certain embodiments one or more components of the compositions target the sensitizer to a specific target (e.g., microorganism, cell type, and the like). In certain embodiments compositions herein are directed to killing microorganisms and/or cells and/or to bleaching colored compounds and/or to increasing the rate of wound healing.

In certain embodiments the sensitizer composition produces an increased concentration of RCS (including ROS), and/or their precursors and/or derivatives, and/or concentration of available. For example, in certain embodiments the concentrations of RCS (e.g., hydrogen peroxide) and available oxygen in the sensitizer solution are increased by increasing the partial pressure of oxygen (e.g., increase the concentration of oxygen in the gas and/or increase the pressure of the gas) in a gas in contact with the sensitizer solution. In certain embodiments the sensitizer solution includes a solubility-increasing compound (e.g., perfluorocarbon and/or hemoglobin, their derivatives and/or precursors). In certain embodiments the sensitizer composition includes production-increasing compounds, activation compound (e.g., catalyst), bleaching agents or combinations thereof. In certain embodiments the sensitizer composition targets general targets or specific targets (e.g., a microorganism or a cell type or a colored compound).

A method for cleaning a biological surface or other tissue including orally dispensing a sensitizer composition under pressure is disclosed herein. In certain embodiments the sensitizer composition is delivered, under pressure, to a treatment site. In certain embodiments the oxygen content of the sensitizer composition is increased before, during, or after, delivery to the treatment site. In certain embodiments electromagnetic energy and/or ultrasound energy and/or thermal energy and/or electrical energy and/or an electric field and/or a magnetic field are delivered and/or applied to the treatment site to activate or otherwise aid in the performance and/or distribution of the sensitizer composition before composition delivery, and/or during composition delivery, and/or after composition delivery.

A method for cleaning a biological surface or other tissue including dispensing a sensitizer composition under pressure is disclosed herein. In certain embodiments the sensitizer composition is delivered, under pressure, to an applicator (e.g., a mouthpiece, a flexible planar surface, or a cleaning device) and/or directly to a treatment site. In certain embodiments the oxygen content of the sensitizer composition is increased before, during, or after, delivery to the target site. In certain embodiments electromagnetic energy and/or ultrasound energy and/or thermal energy and/or electrical energy and/or an electric field and/or a magnetic field are delivered and/or applied to the treatment site to activate or otherwise aid in the performance and/or distribution of the sensitizer composition during composition delivery, after composition delivery, or both.

A method for cleaning a biological surface or other tissue including dispensing a sensitizer composition under pressure onto or through an applicator and then applying the applicator and/or sensitizer solution to a treatment site is disclosed. In certain embodiments the oxygen content of the sensitizer composition is increased before, during, or after, delivery to the applicator and/or target site. In certain embodiments electromagnetic energy and/or ultrasound energy and/or thermal energy and/or electrical energy and/or an electric field and/or a magnetic field are delivered and/or applied to the treatment site to activate or otherwise aid in the performance and/or distribution of the sensitizer composition before composition delivery, and/or during composition delivery, and/or after composition delivery.

A method is disclosed for cleaning a biological surface or other tissue including dispensing a sensitizer composition onto an applicator and then applying the applicator and/or sensitizer solution to a target site. In certain embodiments the oxygen content of the sensitizer composition is increased before, during, or after delivery to the applicator and/or target site. In certain embodiments electromagnetic energy and/or ultrasound energy and/or thermal energy and/or electrical energy and/or an electric field and/or a magnetic field are delivered and/or applied to the treatment site to activate or otherwise aid in the performance and/or distribution of the sensitizer composition before composition delivery, and/or during composition delivery, and/or after composition delivery.

A method for cleaning a non-biological surface including dispensing a sensitizer composition onto an applicator and then applying the applicator to the target site is disclosed. In certain embodiments the oxygen content of the sensitizer composition is increased before, during, or after delivery to the applicator and/or the target site. In certain embodiments electromagnetic energy and/or ultrasound energy and/or thermal energy and/or electrical energy and/or an electric field and/or a magnetic field are delivered and/or applied to the treatment site to activate or otherwise aid in the performance and/or distribution of the sensitizer composition before composition delivery, and/or during composition delivery, and/or after composition delivery.

A method for cleaning a non-biological surface including dispensing a sensitizer composition under pressure is disclosed herein. The sensitizer composition is delivered, under pressure, to a target site. In certain embodiments the oxygen content of the sensitizer composition is increased before during or after delivery to the target site. In certain embodiments electromagnetic energy and/or ultrasound energy and/or thermal energy and/or electrical energy and/or an electric field and/or a magnetic field are delivered and/or applied to the treatment site to activate or otherwise aid in the performance and/or distribution of the sensitizer composition before composition delivery, and/or during composition delivery, and/or after composition delivery.

A method of treating sepsis and/or cancer, includes systemically delivering a therapeutically effective amount of sensitizer solution. The sensitizer and/or a component therein having a high specificity for the sepsis and/or cancerous cells is disclosed herein. In certain embodiments the method includes waiting after delivery for an appropriate time period for absorption or close association (e.g., bound through an antibody, or non-pair member moiety) of the sensitizer by the sepsis and/or cancerous cells and/or for clearance of excess sensitizer. In certain embodiments the method includes applying electromagnetic and/or ultrasound and/or thermal and/or electrical energy and/or an electric field and/or a magnetic field to a target site. In certain embodiments the applied energy is of a type and characteristic (i.e., emission profile) that allows the applied energy to penetrate to and/or through the target site and activate and/or otherwise aid in the performance and/or distribution of the sensitizer composition.

A method of diagnosing sepsis or cancer is disclosed, which method includes applying the sensitizer solution through an appropriate method (e.g., oral, parenteral, including injection, or topical) wherein the sensitizer solution has a high specificity for the sepsis (e.g., microorganisms) and/or cancerous cells. The sensitizer solution emits a detectable wavelength of RF energy when activated by a particular stimulation wavelength of energy. In certain embodiments the method includes allowing an appropriate time period for absorption and/or close association (e.g., bound through an antibody, or non-pair member moiety) of the sensitizer by the sepsis and/or cancerous cells, and/or for clearance of excess sensitizer. In certain embodiments the method includes activating the administered sensitizer with the particular stimulation energy wavelength and detecting the sensitizer's emitted wavelength of radiofrequency (RF) energy.

A method of delivering the sensitizer solutions to a treatment site is disclosed. The method includes incorporating one or more solution containers and/or pressurized cartridges into a dental device or system (e.g., oral irrigation equipment, rinse equipment, drill, ultrasonic scaler, probe), wound care device or system (e.g., wound irrigation device), laparoscopic and/or arthroscopic surgical device or system (e.g., irrigation device), liquid ventilator device or system (e.g., ventilator used for total liquid ventilation of the lungs), mechanical gas ventilator device or system (e.g., ventilator used for gas and/or partial liquid ventilation of the lungs), drug delivery device or system, for example transdermal delivery devices, or combinations thereof.

A method of treating a patient with liquid ventilation is disclosed. The method includes delivering the sensitizer solution to the lung of the patient through total and/or partial liquid ventilation of the lung. In certain embodiments the method includes waiting after delivery for an appropriate time period for absorption and/or close association (e.g., bound through an antibody, or non-pair member moiety) of the sensitizer by a target site (e.g., organism and/or tissue) and/or for clearance of excess sensitizer. In certain embodiments the method further applies electromagnetic and/or ultrasound and/or thermal and/or electrical energy and/or an electric field and/or a magnetic field to a target site on the patient. In certain embodiments the applied energy is of a type and characteristic (i.e., emission profile) so that the applied energy penetrates the patient and activates and/or otherwise aids in the performance and/or distribution of the sensitizer composition.

Methods for detecting and/or killing microorganisms in a volume of liquid are disclosed. The methods include adding the sensitizer composition to the volume of liquid. In certain embodiments the method further comprises waiting for a period of time after adding the sensitizer composition. In certain embodiments the method further comprises applying electromagnetic and/or ultrasound and/or thermal and/or electrical energy and/or an electric field and/or a magnetic field to the volume of liquid. In certain embodiments the applied energy produces an energy emission profile that allows it to sufficiently penetrate the liquid and activate and/or otherwise aid in the performance and/or distribution of the sensitizer composition.

### BRIEF DESCRIPTION OF THE DRAWINGS

For these figures, similar components from one figure to another are considered to have a similar or identical function.

FIG. 1 is a schematic representation of one embodiment of the cleaning system.

FIG. 2A is a graphic representation of spectral absorption of light for two embodiments of the sensitizer solution plotting absorbance versus wavelength in nm.

FIG. 2B is a graphic representation of the spectral absorption of light for four embodiments of the sensitizer solution plotting absorbance versus wavelength in nm.

FIG. 3 is a schematic representation of a partial cutaway isometric view of an embodiment having an internal bladder.

FIG. 4 is a schematic representation of a partial cut-away isometric view of one fluid delivery embodiment having direct pressurized fluid delivery.

FIG. 5 is a schematic representation of an isometric view of two pressurized cans wherein the sensitizer is mixed in a co-joined nozzle for immediate delivery.

FIG. 6 is a schematic representation of a partial cut-away view of a fluid delivery system in a single container having a first and second flexible bladder.

FIG. 7 is a schematic representation of an isometric view of an embodiment utilizing a cartridge and pressure applicator.

FIG. 8 is a schematic representation of an isometric view of a fluid delivery system in conjunction with various applicators including a wand, a wafer, and a mouthpiece.

FIG. 9 is a schematic representation of an isometric view of a fluid delivery system having a light control device and an illumination conduit.

FIG. 10 is similar to FIG. 9 except that the delivery conduit has an offset neck and nozzle that is fixed or removably detachable.

FIG. 11 is a schematic representation of an isometric view of the fluid delivery system having a fluid control and light control.

FIG. 12 is a schematic representation in partial isometric view of the fluid delivery system having various fluid controls and light controls.

FIG. 13 is a schematic representation in isometric view of a bladder container without a rigid cartridge.

FIG. 14 is a schematic representation in isometric view similar to FIG. 7 showing a fluid delivery outlet and light source control.

FIG. 15 is a schematic representation in isometric view similar to FIG. 14 having a shaped delivery body.

FIG. 16 is a schematic representation in partial cut-away isometric view similar to FIG. 15 showing the connecting plate, fluid outlets, and light sources and control.

FIG. 17 is a schematic representation in partial isometric view of an applicator (see FIG. 16) having permanent and electromagnets for use with a fluid having magnetic susceptibility influenced by a flowing current.

FIG. 18 is a schematic representation similar to FIG. 17 having the electrical current flowing in the opposite direction.

FIG. 19 is a schematic representation in isometric view of a fluid cleaning system designed for direct surface application.

FIG. 20 is a schematic representation in isometric view of one embodiment of the fluid delivery system and/or the fluid cleaning system having a solution delivery system (SDS).

FIG. 21 is a schematic representation in isometric view similar to FIG. 20 showing a light source with the fluid outlet.

FIG. 22 is a schematic representation in isometric view similar to FIG. 21 wherein the reservoir has a cover imparting its own properties.

FIG. 23 is a schematic representation in isometric view similar to FIG. 22 wherein the reservoir has separate chambers for liquids, which are mixed and then applied.

FIG. 24 is a schematic representation in isometric view similar to FIGS. 22 and 23 having a cartridge.

FIG. 25 is a schematic representation in isometric view similar to FIG. 24 wherein the SDS includes a concurrent fluid intake conduit.

FIG. 26 is a schematic representation in isometric view wherein the SDS is in fluid communication with the cartridge and delivery conduit.

FIG. 27 is a schematic representation in isometric view similar to FIG. 26 showing a concurrent fluid intake conduit.

FIG. 28 is a schematic representation of the connection and interaction of the components of one embodiment.

FIG. 29 is a schematic representation similar to FIG. 28 of the conditions and interactions wherein a vacuum is created as fluid flows through the venturi tube.

FIG. 30 is a schematic representation similar to FIG. 29 wherein the cleaning system has an external vacuum source.

FIG. 31 is a schematic representation similar to FIG. 30 wherein the cleaning system has a fixed vacuum pump.

FIG. 32 is a schematic representation similar to FIG. 30 wherein the first fluid source and/or the second fluid source are in direct communication with the vacuum pump.

FIG. 33 is a schematic representation similar to FIG. 32 wherein a first pump and a second pump are present.

FIG. 34 is a schematic representation in partial isometric view of a delivery conduit having multiple channels.

FIG. 35 is a schematic representation similar to FIG. 34 having an energy transport device.

FIG. 36 is a schematic representation similar to FIG. 35 having a first and second energy transport device.

FIG. 37 is a schematic representation in isometric view showing a different embodiment of the delivery conduits.

FIG. 38 is a schematic representation in isometric view that shows one or more controls for the power and/or the fluid.

FIG. 39 is a schematic representation of a delivery conduit (applicator).

FIG. 40 is a schematic representation of a partial isometric view of a delivery conduit showing a light source and fluid outlet.

FIG. 41 is a schematic representation of an application similar to FIG. 40 showing a flexible neck.

FIG. 42 is a schematic representation of an application similar to FIGS. 40 and 41 having a flexible neck with a light source and a fluid outlet.

FIG. 43 is a schematic representation of an applicator similar to FIGS. 40-42 having a segmented flexible neck.

FIG. 44 is a schematic representation in isometric view showing the applicator has a flat surface with light source and fluid outlet.

FIG. 45 is a schematic representation similar to FIG. 41 showing that the applicator face is curved.

FIG. 46 is a schematic representation similar to FIG. 45 showing the applicator has a v-shape.

FIG. 47 is a schematic representation showing the applicator having two articulating faces.

FIG. 48 is a schematic representation of a top view of an applicator as a sheet with an active side and an exterior side.

FIG. 49 is a schematic representation in isometric view of a cross-section of FIG. 48 along line A-A.

FIG. 50 is a schematic representation of the sensitizer system having two or more components.

FIG. 51 is a schematic representation in cross-sectional view of the sensitizer solution along axis B-B of FIG. 50 having two or more components.

FIG. 52 is a schematic representation in angled front view of a multi-component sensitizer solution on a flexible applicator.

FIG. 53 is a schematic representation of an applicator sheet for the fluid delivery system.

FIG. 54 is a schematic representation in cross-sectional view at axis C-C of FIG. 53 of the applicator sheet.

FIG. 55 is a schematic representation in isometric view of a mouthpiece applicator having transducers and light sources.

FIG. 56 is a schematic representation in isometric view of a mouthpiece applicator fluid inlets and vacuum removal.

FIG. 57 is a schematic representation in general cross-sectional view of FIG. 55 showing the motion of the charged species.

FIG. 58 is a schematic representation in general cross-sectional view of FIG. 55 showing multiple field lines.

FIG. 59 is a schematic representation in isometric view showing the mouthpiece having multiple openings.

FIG. 60 is a schematic representation in isometric view of a mouthpiece having a notched structure.

FIG. 61 is a schematic representation in isometric view showing that the mouthpiece (bite panel) having multiple light sources.

FIG. 62 is a schematic representation in isometric view showing that the mouthpiece has one or more fluid inlets and light sources.

FIG. 63 is a schematic representation in isometric view of the mouthpiece as a plain surface having light sources and/or fluid inlets.

FIG. 64A is a schematic representation of a mouthpiece configured as a sidewall.

FIG. 64B is a schematic representation in cross-sectional view shows a light source on the lingual side.

FIG. 64C is a schematic representation in cross-sectional view showing multiple light sources.

FIG. 64D is a schematic representation in cross-sectional view showing that the mouthpiece has light sources that extend over multiple surfaces.

FIG. 64E is a schematic representation in cross-sectional view having a mouthpiece with one or more diffusers.

FIG. 65 is a schematic representation in isometric view of a mouthpiece having a bite panel and a single sidewall and lingual wall.

FIG. 66 is a schematic representation in isometric view of a mouthpiece having a sidewall top and bottom.

FIG. 67 is a schematic representation in isometric view having a mouthpiece attached to a palate panel.

FIG. 68 is a schematic representation in isometric view of the mouthpiece (palate of FIG. 67) connected to a power source.

FIG. 69 is a schematic representation in isometric view of the mouthpiece of FIG. 67 further including transducers, fluid outlets, and light sources.

FIG. 70 is a schematic representation in isometric view of the mouthpiece/palate of FIG. 69 connected to a power source.

FIG. 71 is a schematic representation in isometric view of a mouthpiece (bite panel), handle, and power source.

FIG. 72 is a schematic representation in isometric view of a bite panel and applicator similar to FIG. 71.

FIG. 73 is a schematic representation in isometric view of a bite block of an applicator similar to FIG. 72.

FIG. 74 is a schematic representation in isometric view of an applicator wherein the bite block is adjustable.

FIG. 75 is a schematic representation in isometric view of a bite block.

FIG. 76 is a schematic representation in isometric view of a hand held applicator for use with a patient.

FIG. 77 is a schematic representation in isometric view of FIG. 76 showing controls for transducers, fluid outlets, illuminators, etc.

FIG. 78 is a schematic representation in isometric view showing the applicator as a catheter using a balloon.

FIG. 79 is a schematic representation in isometric view showing the applicator as a catheter having two balloons.

FIG. 80 is an enlargement of the distal end of the applicator of FIG. 79.

FIG. 81 is a schematic representation in isometric view of the cleaning system configured as a bath or soaking device.

FIG. 82 is an enlargement of the tip of FIG. 81 having one or more fluid outlets and optionally light sources.

FIG. 83 is a schematic representation in cross-sectional view of a method of cleaning a tooth site in need of treatment.

FIG. 84 is a schematic representation in cross-sectional view of the method of cleaning tooth site by direct application of solution.

FIG. 85 is a schematic representation in cross-sectional view of a tooth site being cleaned by application of the solution and illumination.

FIG. 86 is a schematic representation in cross-sectional view of a tooth having sepsis on the exterior.

FIG. 87 is a schematic representation in cross-sectional view of sepsis on the gingiva surface being treated by the method and apparatus of the present invention.

FIG. 88 is a schematic representation in cross-sectional view of a tooth extraction site being cleaned by the method and apparatus of the present invention.

FIG. 89 is a schematic representation in cross-sectional view of a mouth wherein the tooth/gingiva site is being cleaned.

FIG. 90 is a schematic representation of a mouth where the tooth site is being treated using an applicator.

FIG. 91 is a schematic representation in cross-sectional view of a tooth being treated according to FIG. 90.

FIG. 92 is a schematic representation in isometric view of a mouthpiece in place over the upper teeth.

FIG. 93 is a schematic representation in cross-sectional view of a tooth, gum infection, and mouthpiece with its components.

FIG. 94 is a schematic representation in isometric view of teeth with a bite block.

### DETAILED DESCRIPTION OF THE INVENTION

### Definitions

As used herein:
“Antibiotics” refers to antibiotics generally known in the art, some specific antibiotics contemplated are; Aminoglycoside antibiotics, e.g., Aminoglycoside, Amikacin, Apramycin, Capreomycin, Ertapenem, Faropenem, Gentamicin, Imipenem, Kanamycin, Neomycin, Paromomycin sulfate, Streptomycin, Tobramycin; Beta-lactam antibiotics, e.g., Carbapenem antibiotics, e.g., Carbapenem, Doripenem, Ertapenem, Faropenem, Imipenem, Meropenem; Cephalosporin antibiotics, e.g., Cephalosporin, Carbacephem, Cefacetrile, Cefaclor, Cefadroxil, Cefalexin, Cefalotin, Cefazolin, Cefdinir, Cefepime, Cefixime, Cefinenoxime, Cefotaxime, Cefotetan, Cefotiam, Cefpodoxime, Cefprozil, Cefradine, Ceftazidime, Ceftriaxone, Cefuroxime, Cephamycin; Loracarbef; Monobactam antibiotics, e.g., Aztreonam; Beta-lactamase inhibitors, e.g., Clavulanic acid, Potassium clavulanate, Tazobactam; Combination antibiotics, e.g., Ampicillin/sulbactam, Co-amoxiclav, Co-fluampicil, Co-trimoxazole, Imipenem/cilastatin, Neosporin, Piperacillin/tazobactam, Quinupristin/dalfopristin; Fluoroquinolone antibiotics, e.g., Quinolone, Ciprofloxacin, Enoxacin, Gatifloxacin, Gemifloxacin, Grepafloxacin, Levofloxacin, Lomefloxacin, Marbofloxacin, Nalidixic acid, Norfloxacin, Ofloxacin, Sparfloxacin, Trovafloxacin; Glycopeptide antibiotics, e.g., Nonribosomal peptide, Teicoplanin, Vancomycin, ramoplanin, and decaplanin; Glycylcycline antibiotics, e.g., Tigecycline; Lincosamide antibiotics, e.g., Clindamycin, Lincomycin, Lincosamides; Nitroimidazole antibiotics, Nitroimidazole, Metronidazole; Polyketide antibiotics; Macrolide antibiotics, e.g., Macrolide, Azithromycin, Brefeldin A, Clarithromycin, Dirithromycin, Erythromycin, Ketolides, Roxithromycin, Tacrolimus, Telithromycin; Tetracycline antibiotics, e.g., Chlortetracycline, Demeclocycline, Doxycycline, Lymecycline, Minocycline, Oxytetracycline, Periostat, Tetracycline; Polymyxin antibiotics, e.g., Colistin, Polymyxin; Polypeptide antibiotics, e.g., Lantibiotics, Nisin; Rifamycin antibiotics, e.g., Rifabutin, Rifampicin, Rifamycin, Rifapentine, Rifaximin; Sulfonamide antibiotics, e.g., Sulfonamides, Aldesulfone sodium, Dapsone, Prontosil, Sulfamethoxazole, and Sulfisoxazole. Actinomycin, Antistaphylococcal penicillins, Azlocillin, Bacitracin, Cefaloglycin, Cefapirin, Ceforanide, Cefpiramide, Cinoxacin, Cloxacillin, Ethambutol, Furazolidone, Hetacillin, Hitachimycin, Monobactams, Myriocin, Nafcillin, Oxacillin, Penicillin, Puromycin, Sulfamethizole, Sultamicillin, Thioacetazone, Viomycin, Spectinomycin, Trimethoprim, and Chloramphenicol.
“Applicator” refers to any device used to apply the composition and light energy.
“Illuminator” or “light source” refers to any electromagnetic radiation source, or any vibrational energy source, or any magnetic or electric field energy source.
“Is,” “are,” “have,” “had,” and similar verbs are normally to be interpreted as associated with the term “in this embodiment” or with “optionally.” Thus, where choices or options are shown, the invention has the described or doesn't have the described feature, but the invention also functions with other components and embodiments.
“Oxidative reductive potential water solution” is synonymous with our term “reactive chemical species” and refers to the general and specific aqueous solutions as described above in US Patent Publications 2005/1039808 A1, 2005/0142157 A1 and 2005/0196462 A1. The uses of these solutions are contemplated in the present invention in combination with 1) gas solubility-increasing compounds such as perfluorocarbons, 2) elevated pressure, 3) concentrations of oxygen at or above those found at ambient conditions 4) photosensitizing solutions in combination with electromagnetic radiation, and 5) combinations of #1 to 4 thereof.
“PDT” or “photodynamic therapy” refers to a specific form of phototherapy that utilizes a photosensitizer (a molecule having the ability to absorb light energy and then use this energy to carry out chemical reactions).
“RCS” or “reactive chemical species” refers to those energetic chemical species that are responsible for toxic properties to kill microspecies and pathogens, and to bleach colored compounds, e.g., hydrogen peroxide.
“ROS” or “reactive oxygen species” refer to specific RCS of oxygen, e.g., singlet oxygen, hydrogen peroxide, ozone, etc.
“SDT” or “sonodynamic therapy” utilizes a sonosensitizer (a molecule having the ability to absorb vibrational energy and then use this energy to carry out chemical reactions).
“Sensitizer solution” refers to those solutions as described herein. It is contemplated that some sensitizer solutions optionally will have a sensitizer.
“Target” or “target area” refers to the area being treated according to the current invention.

Other definitions of terms appear in the subsequent text.

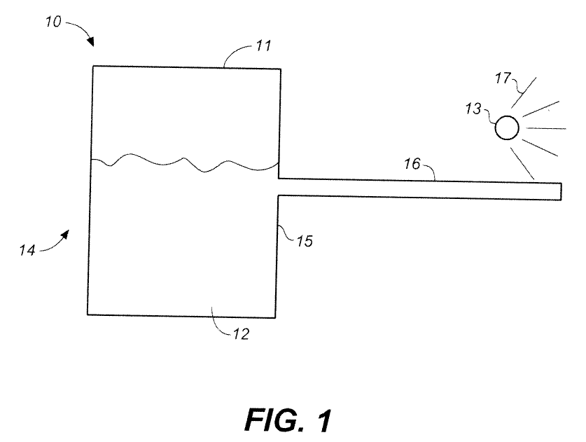

With reference to the figures, FIG. 1 illustrates an embodiment of a cleaning system 10. In certain embodiments the cleaning system 10 has a fluid source 11, sensitizer solution 12, and a transducer 13. In certain embodiments the sensitizer solution 12 is a sonosensitizer solution, a photosensitizer solution or combinations thereof. The sensitizer solution 12 is in the fluid delivery system 14, for example inside of a fluid container 15. The sensitizer solution 12 is in a flowable or optionally non-flowable form. In certain embodiments the sensitizer solution 12 is also a composition. A composition is an aqueous or non-aqueous solution, suspension, or dispersion such as a liquid or solid aerosol, foam, gel, emulsion (e.g., oil-in-water, water-in-oil, etc.), paste, powder, solid, crystal, micelle, liquid crystal, sols, sol gel, semisolid or macroscopic suspension, or combinations thereof. In certain embodiments the composition is in, or contains one or more components in, a microencapsulated form such as alginate beads or agar gel beads, liposomes, niosomes, particles (e.g., macro, micro and/or nano scale particles and/or spheres (e.g., microspheres, such as albumin microspheres, and/or crystals) or other form in which a boundary layer is formed to surround the sensitizer and/or other components of the sensitizer solution. Such formulations are known in the art, for example as disclosed in U.S. Pat. Nos. 6,375,985; 6,375,968; 6,319,507; 6,217,908, and Microencapsulation: Methods and Industrial Applications in Drugs and the Pharmaceutical Sciences, Vol. 73; S. Benita (Ed.); Marcel Dekker; 1996. A time-release formulation is also contemplated, such as that disclosed in U.S. Pat. No. 6,197,331.

The fluid source 15 is selected from a container, a cartridge, a substantially unbreakable or breakable ampoule, a syringe (e.g., a plunger-type mechanism), a bag, a tube, a reservoir, a squeeze bulb, an external supply such as a well-fed or municipal water supply, or combinations thereof. The fluid source 15 may or may not be pressurized.

The transducer 13 is separate from or integral with the fluid source 15. The transducer 15 emits energy 17. The transducer has an energy emission profile. The energy emission profile is defined by all relevant characteristics of the energy emission, for example energy type, frequency, intensity, pulse duration, orientation, polarity, pulse repetition rate (frequency) and/or a characteristics rate of change. The energy profile 17 is selected from electromagnetic energy such as ultraviolet, visible, near infrared, infrared, microwave, radio, X-ray and nuclear magnetic resonance, vibration energy such as acoustic energy and/or ultrasonic energy, or combinations thereof.

In certain embodiments the transducer 13 is an electromagnetic emitting transducer, for example an RF emitting transducer. For example, the RF emitting transducer emits infrared (IR), and/or near infrared, and/or ultraviolet (UV), and/or visible light, and/or microwave, and/or radio, and/or x-ray energy. In certain embodiments the transducer 13 is configured to emit light energy, such as an illuminating device. In certain embodiments the transducer 13 emits RF energy in the visible spectrum. In certain embodiments the transducer 13 is configured to emit light energy 17. In certain embodiments the illuminating device emits RF energy in the non-visible spectrum. In certain embodiments the transducer 13 is selected from an LED, Laser diode, laser, x-ray source, RF generator, microwave generator, positron source, electron beam generator, and/or nuclear magnetic resonance machine.

The transducer 13 is optionally also a thermal device, such as a heating and/or cooling device. In certain embodiments the thermal device is a heating coil, an electrical resistor, a peltier thermoelectric device, or combinations thereof.

The transducer 13 is optionally also a vibrating device, acoustic source, and/or ultrasonic energy-producing device (e.g., which is used with a sonosensitizer solution in lieu of or in combination with the photosensitizer solution).

In certain embodiments the transducer 13 is configured to have an adjustable energy emission profile. For example, transducer 13 is configured to have an adjustable frequency (i.e., wavelength) and/or intensity level. In certain embodiments the transducer is configured to have a continuous and/or discontinuous (i.e., pulsatile or strobing) emission duration at a preset and/or adjustable repetition rate. In certain embodiments the emission duration has a regular and/or irregular repetition rate. In certain embodiments the transducer 13 is configured, for example through the use of a microprocessor, to have an energy emission profile, in which the characteristics of the energy emission vary as a function of time. In certain embodiments the feedback from sensors is used to automatically adjust the transducers energy emission profile, and/or to alert the user of recommended actions, for example through the sounding of a tone and/or the flashing of a light. In certain embodiments the cleaning system is configured to allow the user to choose if the energy emission profile is adjusted manually or automatically through, for example, a switch.

The transducer emits an energy dosage, for example a light dosage, ranging from about 0.5 J/cm² to about 300 J/cm², and/or from about 5 J/cm² to about 30 J/cm², and/or from about 30 J/cm² to about 60 J/cm², and/or from about 60 J/cm² to about 100 J/cm², and/or from about 100 J/cm² to about 200 J/cm², and/or from about 200 J/cm² to about 300 J/cm². In certain embodiments the transducer emits a power density from about 0.1 mW/cm² to about 300 mW/cm². In certain embodiments the transducer 13 emits energy for a duration from about 100 nsec to about 24 hours. In certain embodiments the transducer 13 emits energy 17 at a repetition rate, for example, from about 0.01 Hz to about 10 kHz. In certain embodiments the transducer 13 is configured to emit the energy 17 at a frequency and/or intensity, and/or repetition rate that would substantially activate the sensitizer solution 12. For example, transducer 13 is configured to emit the electromagnetic energy 17 at a frequency and/or intensity and/or duration and/or repetition rate that would substantially activate the photosensitizer solution 12. Also for example, transducer 13 is configured to emit the acoustic energy 17 at a frequency and/or intensity and/or duration and/or repetition rate that would substantially activate the sonosensitizer solution 12. The therapeutic use of light energy alone, for example low level laser therapy (LLLT), or low level laser biostimulation (LLLB), is well known to those skilled in the art, and has been shown to induce changes in an organism that lead to healing, pain reduction, increased rate of cellular attachment to implants, and/or destruction of bacteria, cancer, and viruses. In certain embodiments the sensitizer activation transducer, and/or one or more separate transducers dedicated to this purpose, can emit light energy as called for by therapeutic applications of LLLT and LLLB. As an example of LLLT, U.S. Pat. No. 5,658,148, by Neuberger et al., discloses the use of a GaAs pulsed diode laser at a pulse width of 200-300 nsec, wavelength of 904 nm, power of 5-10 mW and application duration of 1-3 minutes with the following 3 different pulse frequencies to treat specific conditions: F1=73 Hz for parodontitides, F2=292 Hz for gingivitis and stomatitis, F3=584 Hz for gingivitis, stomatitis paradontophaties. L. J. Walsh reviews several LLLT applications for both soft and hard tissues in “The current status of low level laser therapy in dentistry. Part 1. Soft tissue applications”, L. J. Walsh, Australian Dental Journal 1997; 42:(4):247-54 and “The current status of low level laser therapy in dentistry. Part 2. Hard tissue Applications”, L. J. Walsh, Australian Dental Journal 1997; 42:(5):302-6, both of which are incorporated by reference herein in their entirety.

In certain embodiments transducer 13 emits vibratory energy 17 at a frequency, for example, between 5 kHz and 12 MHz, more narrowly between about 50 kHz and 5 MHz, yet more narrowly between about 1 MHz and 3 MHz. In certain embodiments the vibratory energy has an intensity range of from about 0.05 to about 80 W/cm², for example from about 0.05 W/cm² to about 0.25 W/cm², and/or from about 0.25 W/cm² to about 3 W/cm², and/or from about 3 W/cm² to about 10 W/cm², and/or from about 10 W/cm² to about 20 W/cm², and/or from about 20 W/cm² to about 40 W/cm², and/or from about 40 W/cm² to about 60 W/cm², and/or from about 60 W/cm² to about 80 W/cm². U.S. Pat. No. 6,627,664, by Miller et al. The energy emission profile is preset and/or adjustable depending on the selection of the sensitizer solution 12. The ability of the user to choose how the cleaning systems energy emission profile is controlled (e.g., whether it is automatic or manual), is dependent on the selection of the sensitizer solution 12 and/or requirements of the application. In certain embodiments the cleaning system is configured so that the acoustic energy produces a pressure gradient that results in the sensitizer solution flowing down (i.e., from a region of higher pressure to a region of lower pressure) the pressure gradient. In certain embodiments the cleaning system is configured so that the pattern of flow is recirculating.

The sensitizer solution 12 is in any suitable form and/or composition. The particular sensitizer solution composition to be used will depend on the intended method of administration, whether the mode of administration is oral, parenteral, including injection, or topical, and the like, for example. During shipping and/or storage and/or delivery and/or use the sensitizer solution 12 is in a form that is flowable, for example, an aqueous or non-aqueous solution, suspension, or dispersion such as a liquid or solid aerosol, foam, gel, emulsion (e.g., oil-in-water, water-in-oil), paste, powder, micelle, liquid crystal, liposome, sols, sol gel, semisolid or macrosolid suspension or combinations thereof. Details on how to prepare many of these forms are provided in Remington's Pharmaceutical Sciences, 18th ed. 1990, which is hereby incorporated by reference in its entirety.

The sensitizer solution in one embodiment is in a non-flowable form, for example a solid, or crystal. In certain embodiments the sensitizer solution 12 is a pharmaceutically acceptable composition, for example, the proportion and nature of which is determined by the solubility and chemical properties of the sensitizer selected, the chosen route of administration and standard pharmaceutical practice. For example, in certain embodiments the sensitizer solution 12 is made from a pharmaceutically acceptable sensitizer mixed with a pharmaceutically acceptable aqueous carrier. In certain embodiments the aqueous carrier is water such as distilled water, demineralized water, pyrogen-free water, sterile water, or water having combinations of the aforementioned characteristics. “Pharmaceutically acceptable” is acceptable to be included as a component of a composition that comes in contact with a living organism. A sensitizer solution utilized in accordance with the teachings herein is administered in any form or mode that makes the sensitizer available to participate in the ways described herein, including oral, parenteral, and topical routes. A non-exclusive list of administration routes includes, oral, subcutaneous, intramuscular, intravenous, transdermal, intranasal, rectal, and topical routes.

Sensitizer 12 is any compound that absorbs the energy 17 to reach an excited state that can then undergo further reactions. In certain embodiments the sensitizer is a photosensitizer and/or a sonosensitizer. The photosensitizer is a compound that reaches an activated state through the absorption of electromagnetic energy, for example light. The sonosensitizer is a compound that reaches an activated state through the absorption of acoustic energy, for example, ultrasound and/or sonoluminescence. Some compounds are photosensitizers and sonosensitizers. The excited state of the sensitizer directly participates in a reaction with a substrate (Type I reaction), or reacts with oxygen in the triplet (ground) state to produce singlet (excited state) oxygen or superoxide anion (Type II reaction). “DNA Damage and Cell Lethality by Photodynamically Produced Oxygen Radicals”, Paula Burch, Ph.D. Thesis, Rice University, 1989 is incorporated by reference herein in its entirety.

Sensitizers are those sensitizers known to those having skill in the art to be cytotoxic when illuminated with electromagnetic and/or acoustic energy of a particular intensity and/or wavelength or combination of wavelengths. Sensitizers are those sensitizers known to those having skill in the art to produce singlet oxygen upon absorption of electromagnetic and/or acoustic energy at a particular energy intensity and/or wavelength or combination of wavelengths. Singlet oxygen has direct toxic effects on microorganisms and also undergoes further non-photolytic reactions, for example chemical reactions, to produce other toxic reactive oxygen species (ROS), for example hydroxyl radical, superoxide anion, peroxides (e.g., (H₂O₂), and hypochlorous acid (HOCl), which themselves may have a toxic effect on microorganisms. Singlet oxygen undergoes a chemical reaction with hydrogen peroxide to produce the hydroxyl radical (OH.) through the Haber-Weiss reaction or through the Fenton reaction if Fe++ and hydrogen peroxide are present together.

ROS refers collectively to oxygen and many of oxygen's reaction products. ROS are toxic in varying degrees to living organisms including those microorganisms discussed herein. In certain embodiments ROS are free radicals, such as superoxide radical (O₂.⁻), the protonated superoxide radical (HO₂.), peroxyl radicals (ROO.), alkoxyl radicals (RO.) and hydroxy radical (OH.). In certain embodiments ROS include oxygen derivatives that do not contain unpaired electrons, such as peroxides (for example, hydrogen peroxide (H₂O₂), carbamide peroxide (also known as urea hydrogen peroxide, hydrogen peroxide carbamide, and perhydrol-urea and available in over the counter compositions as “Gly-Oxide®” by Marion Laboratories, Kansas City, Kans. and “Proxigel” by Reed and Cammick Pharmaceuticals, Jersey City, N.J.), singlet oxygen (1Δg O₂), superoxide anion (O₂⁻), and hypochlorous acid (HOCl). A “derivative” is a compound that is formed from a similar compound.

In certain embodiments the sensitizer solution 12 has an RCS concentration. In certain embodiments the RCS are generally or completely composed of ROS. RCS and ROS mixtures are contemplated. In certain embodiments the sensitizer solution 12 is fully saturated with one or more ROS. In certain embodiments the ROS concentration is increased, for example, by increasing the partial pressure of the ROS in contact with the sensitizer solution 12. In certain embodiments the partial pressure of the ROS is increased, for example, by including a gas containing reactive oxygen species in contact with the sensitizer solution 12; increasing the pressure of the gas in the fluid delivery system 12, for example by increasing the quantity of gas in a fixed volume element of the fluid delivery system 14; and/or increasing the concentration of ROS in the gas, relative to the other components of the gas.

In certain embodiments the ROS concentration in the sensitizer solution 12 is increased, for example, by including a gas in contact with the sensitizer solution 12. In certain embodiments the gas has reactive components. In certain embodiments the reactive components react with components in the sensitizer solution to produce ROS. In certain embodiments the ROS concentration in the sensitizer solution is increased by increasing the partial pressure of the reactive components in the gas and/or the concentration of the reactive components in the sensitizer solution. In certain embodiments the partial pressure is increased by increasing the quantity of gas in a fixed volume element of the fluid delivery system 14; and/or increasing the concentration of the reactive components in the gas and/or sensitizer solution, relative to the other components of the gas and sensitizer solution respectively. This phenomenon is described by Le Chatelier's principle. For example, oxygen reacts with water to produce the ROS hydrogen peroxide. In certain embodiments the partial pressure of oxygen in contact with a sensitizer solution that contains water is increased, thereby creating an increased concentration of hydrogen peroxide.

In certain embodiments the sensitizer solution can comprise oxidative reductive potential water as the reactive chemical species.

In certain embodiments the sensitizer solution comprises oxidative reductive potential water solution as described in the incorporated by reference Oculus patent publications. For example, a tooth whitener, for example, a commercially available tooth whitener comprising a hydrogen peroxide concentration of between about 3% up to about 35%, or a concentration of carbamide peroxide of between about 9% up to about 70%, and water concentration of between about 20% up to about 70%, wherein the water concentration, in whole or in part, is replaced by oxidative reductive potential water.

In certain embodiments the sensitizer solution 12 contains a therapeutically effective amount of one or more sensitizers to provide a therapeutic effect for a given condition under a given administration regimen. The therapeutically effective amount is an amount that provides a therapeutic effect for a given condition and administration regimen. The therapeutically effective amount is at least a biostatic amount and/or a biocide amount and/or an insecticide amount.

In certain embodiments sensitizer solution 12 is present in a sensitizer concentration. In certain embodiments the sensitizer concentration is, for example, from about 0.0000001% w/v to about 25% w/v, more narrowly from about 0.0000001% w/v to about 5% w/v, yet more narrowly from about 0.0000001% w/v to about 1% w/v, yet more narrowly from about 0.0001% w/v to about 1% w/v, yet more narrowly from about 0.0001% w/v to about 0.1% w/v, yet more narrowly from about 0.001% w/v to about 0.01% w/v, for example about 0.005% w/v. In certain embodiments the sensitizer concentration is also present from about 0.0000001% w/v to about 0.1% w/v, more narrowly from about 0.0000001% w/v to about 0.01% w/v, yet more narrowly from about 0.0000001% w/v to about 0.001% w/v, yet more narrowly from about 0.0000001% w/v to about 0.0001% w/v, and/or yet more narrowly from about 0.0000001% w/v to about 0.00001% w/v, for example about 0.000005% w/v. We contemplate inorganic compounds as sensitizers (e.g., RCS, ROS, hydrogen peroxide that have a concentration as high as 40%

Examples of sensitizers are disclosed by Wilson in U.S. Pat. No. 5,611,793, by Hasan in U.S. Pat. No. 6,462,070, by Miller in U.S. Pat. No. 6,627,664, by Alfheim in U.S. Pat. No. 6,498,945, by Bommer et al. in U.S. Pat. No. 4,977,177, by Pandey et al., in U.S. Pat. No. 5,591,847, U.S. Pat. No. 5,770,730, and Australian Patent, 669,876, by Infrared Absorbing Dyes; Masaru Matsuoka, Ed., Plenum Press, New York, 1990, by Chang et al. in PCT application WO 2004/105860, and by Graf, et al. in U.S. Pat. No. 4,648,992, which describes phthalocyanine compounds and their methods of manufacture, all of which are incorporated herein by reference in their entireties.

The sensitizers are often naturally occurring. The sensitizers and/or sensitizer solutions 12 may contain dyes, cationic dyes, phthalocyanines, naphthalocyanines, pheophorbides, purpurins, natural and modified porphyrins, and porphyrin derivatives (define the term derivatives), naturally occurring plant pigments such as chlorins, and bacteriochlorins, perylenequinones, natural perylenequinonoid pigments (PQP), and their derivatives, analogs, isomers, metabolites, pharmaceutically acceptable salts, pharmaceutical products, hydrates, N-oxides, or any combination thereof. Isomers include optical isomers and analogs, structural isomers and analogs, conformational isomers and analogs, combinations thereof.

Additional examples of sensitizers include, but are not limited to, toluidine blue, toluidine blue O, rose bengal, neutral red, arianor steel blue, tryptan blue, crystal violet, methylene blue, fluorescein, xanthenes, thiazines, acridines (e.g., acridines orange, acridines yellow), flavins (e.g., proflavin, riboflavin), azure blue cert, azure B chloride, azure 2, azure A chloride, azure B tetrafluoroborate, thionin, psoralens, psoralens with UVA, benzoyl peroxide, azure A eosinate, azure B Eosinate, azure mix sicc., azure II eosinate, 5-aminolaevulinic acid (ALA), haematoporphyrin HCl, haematoporphyrin ester, benzoporphyrin derivatives, meso-substituted porphyrins, Erythrosin B, Hypericin, benzol-alphenoxazinium dyes, benzo[a]phenothiazinium dyes, kryptocyaine dyes, tellurapyrylium dyes, phenothiazines, chalcogenapyrylium dyes, metalated phthalocyanines (e.g., Zn (II) phthalocyanine, aluminum tetrasulfonated phthalocyanine, zinc tetrasulfonated phthalocyanine, silicon tetrasulfonated phthalocyanine and aluminum disulphonated phthalocyanine), sulfonated phthalocyanines, hydroxylated phthalocyanines, alkoxylated phthalocyanines, metalated naphthalocyanines (e.g., silicon naphthalocyanine, zinc naphthalocyanine, aluminum naphthalocyanine, and Pd (OBu).sub.8. naphthalocyanine, tetrapyrrole derivatives, chlorin, polylysine-bound chlorin, chlorin e₆, mono-L-aspartyl chlorin e6, mono-L-glutamyl chlorin e6, pheophorbide a, bacteriochlorin a, purpurins (e.g., etiopurpurin (SnET₂), ZnET₂, NT₂H₂), metalated purpurins, Lu-texaphyrin, tetrahydroxyphenylchlorin (THPC), rhodamines (e.g., mitochondria-specific Rhodamine 123), titanium dioxide, and verdins. Examples of sensitizers that are perylenequinones and/or natural perylenequinonoid pigments include hypocrellins (hypocrellin A (HA), and hypocrellin B (HB)), cercosporin, phleichrome, cladochrome, elsinochromes, erythroaphins, and calphostins. Further examples of hypocrellin derivatives include; HA-Mg++, HB-Mg++, Deacetylated-HA, Cystamine-HB, n-butylaminated HB, 2-morpholino-ethyl-aminated-HB, 2-(N,N-diethyl-amino) ethylamine-HB, 2-(N,N-diethyl-amino) propylamine-HB, Ethanolamine-HB, Ethylenediamine-HB, Methylamine-HB, 5,8-dibromo-HB, demethylated HB, 1,12-Bis[2-(acetyloxy)propyl]-2,4,6,7,9,11-hexamethoxy-3,10-perylenedione. The following is a non-exclusive list of photosensitizer brands that the compositions and methods described herein is used with: PHOTOFRIN (QLT, Vancouver, Canada), PHOTOFRIN II (QLT, Vancouver, Canada), PHOTOFLORA, PHOTOSENSE (Russia), PHOTOHEM Russia), VERTEPORFINS (QLT, Vancouver, Canada), LUTRIN (Pharmacyclics, USA), FOSCAN (Biolitec AG, Germany), EVULAN (Dusa Pharmaceuticals, Toronto, Canada), VISUDYNE (QLT and Novartis Opthalmics, Vancouver, Canada, and Duluth, Georgia), METVIX (Photocure, Oslo, Norway), PHOTOPOINT SnET2 (Miravant Medical Technologies, Santa Barbara, Calif.), PHOTOPOINT MV9411 (Miravant Medical Technologies, Santa Barbara, Calif.), ANTRIN (Pharmacyclics, Sunnyvale, Calif.), LUTRIN (Pharmacyclics, Sunnyvale, Calif.).

The sensitizer solution 12 has one or more sensitizers mixed with a carrier. The carrier is one or more pharmaceutically acceptable carriers, solvents, diluents, or combinations thereof. The carrier is a liquid carrier for liquid formulations, a solid carrier for solid formulations, or combinations thereof. Pharmaceutically acceptable liquid carriers include aqueous and/or non-aqueous carriers, or combinations thereof. The carrier optionally is an oil-based carrier.

Examples of aqueous carriers include water such as distilled water, demineralized water, pyrogen-free water, sterile water, oxidative reductive potential water as described in the incorporated by reference Oculus patent publications, or water having combinations of the aforementioned characteristics, alcoholic/aqueous compositions, saline, ringers lactate, buffered media, and combinations thereof. Examples of oil-based carriers include those of petroleum, animal, plant, vegetable, or synthetic origin, for example, peanut oil, soybean oil, mineral oil, olive oil, sunflower oil, flax oil, fish liver oil, and combinations thereof. Examples of pharmaceutically acceptable non-aqueous carriers, include but are not limited to, ethanol, propylene glycol, polyethylene glycol of the liquid series, injectable organic esters such as ethyl oleate, acetone, dimethyl acetamide, dimethyl formamide, dimethyl sulfoxide ethanol, glycerin, polyethylene glycol 300 and 400, sorbitol, polyoxyethylene sorbitan, fatty acid esters such as laureate, palmitate, stearate, and oleate, polyoxyethylated vegetable oil, sorbitan monopalmitate, 2-pyrrolidone; n-methyl-2-pyrrolidine; n-ethyl-1-pyrrolidine; tetrahydrofurfuryl alcohol, TWEEN 80 and dimethyl isosorbide, or combinations thereof. The carrier is both an aqueous and non-aqueous carrier. For example, dimethyl isosorbide (ARLASOLVE®. DMI, ICI Specialty Chemicals, Wilmington, Del.) is both water- and oil-soluble. The carrier is gelled with a gelling agent to produce gel formulations. The gelling agent is, for example, about 4% KLUCEL® by Hercules, Inc., Wilmington, Del. Dimethyl isosorbide is gelled with 4% KLUCEL®.

The solid carriers (e.g., diluents) include a gum, a starch (e.g., corn starch, pregelatinised starch), a sugar (e.g., lactose, mannitol, sucrose, dextrose, fructose, maltose), a cellulosic material (e.g., microcrystalline cellulose), an acrylate (e.g., polymethylacrylate), a gelling agent, calcium carbonate, magnesium oxide, talc, or combinations thereof.

In certain embodiments the sensitizer solution 12 includes ROS and/or precursors of ROS and/or derivatives of ROS. In certain embodiments the sensitizer solution 12 includes peroxides and other peroxy compounds (e.g., hydrogen peroxide, carbamide peroxide, sodium perborate (monohydrate or tetrahydrate), sodium percarbonate).

In certain embodiments the sensitizer solution 12 is divided into two or more parts of differing compositions. The parts of the sensitizer solution 12 are prevented from mixing until a time determined by the user and/or by the design of the fluid delivery system. For example, the parts of the sensitizer solution 12 can flow through separate conduits until the parts reach the desired mixing location. In certain embodiments the desired mixing location is the treatment site. In certain embodiments mixing of the parts of the sensitizer solution 12 results in one or more chemical reactions, and/or a series of chemical reactions, to produce one or more sensitizers, and/or their precursors, and/or their derivatives. In certain embodiments mixing the parts of the sensitizer solution 12 results in one or more chemical reactions to produce singlet oxygen, and/or molecular oxygen, and/or other ROS, and/or precursors of ROS, and/or derivatives of ROS.

Examples of chemical reactions that produce ROS include the following: Combining tetramethyl-ammonium superoxide and/or potassium superoxide with water produces a superoxide anion. The superoxide anion reacts with protons to produce hydrogen peroxide and molecular oxygen. This reaction is catalyzed by the enzyme superoxide dismutase in vivo. Hydrogen peroxide reacts with superoxide anion to produce the hydroxyl free radical (OH.) through the Haber-Weiss reaction. Alternatively, superoxide anion through two steps, by way of the Fenton reaction, produces the hydroxyl free radical (OH.). Hydrogen peroxide is produced in a chemical reaction from sodium perborate (monohydrate and/or tetrahydrate) or carbamide peroxide (e.g., 10% carbamide peroxide releases 3.5% hydrogen peroxide). Oxygen can react with water to produce hydrogen peroxide.

Examples of chemical reactions that produce the ROS singlet oxygen include the following: a Fenton type metal catalyzed reaction between superoxide anion and hydrogen peroxide; a reaction between hypochlorite with hydrogen peroxide; a reaction between superoxide anion and diacyl peroxides; a reaction between superoxide anion and the hydroxyl free, dismutation of the superoxide anion to produce singlet oxygen and hydrogen peroxide.

Singlet oxygen reacts with membrane polyunsaturated fatty acids to form lipid hydroperoxides. Transition metals, such as Fe++, catalyze the production of cytotoxic free radicals from lipid hydroperoxides.

In certain embodiments the sensitizer solution 12 has production-increasing compounds, for example catalysts. In certain embodiments the production-increasing compounds increase the rate of production of RCS, and/or ROS and/or ROS precursors (e.g., hydroxyl free radical (OH.) and/or lipid hydroperoxides) through chemical reactions. In certain embodiments the sensitizer solution 12 has a production-increasing compound concentration. In certain embodiments the production-increasing compound concentration is from about 0.00001% v/v to about 5% v/v.

Examples of production-increasing compounds include ascorbate, free metal ions of transition metals (e.g., iron, copper, manganese), chelated/complexed iron and/or copper, and combinations thereof. Examples of iron chelating/complexing agents include phosphate esters, such as ADP, ATP, GTP and pyrophosphate, succinate pyrophosphate, citrate, oxalate, ethylenediaminetetraacetic acid (EDTA), diethylenetriaminepentaacetic acid (DTPA), dipyridyl, phenanthroline, and nitrilotriacetic acid and their derivatives, and combinations thereof.

Ascorbate reduces Fe+++ to Fe++. Fe++ optionally participates, for example as a catalyst, in additional chemical reactions to produce toxic species. Toxic species include RCS (e.g., ROS) that produce a toxic effect. For example, Fe++ reacts with peroxide to produce hydroxide radical through the Fenton reaction and/or with lipid hydroperoxides to produce cytotoxic free radicals.

In certain embodiments the sensitizer solution 12 contains fluoride compounds, for example to fight tooth decay. Fluoride compounds (fluoride therapies) promote the remineralisation of teeth, making teeth harder and more resistant to the formation of tooth decay, inhibiting oral bacteria's ability to create acids. Examples of fluoride compounds and several exemplary concentration ranges or example concentrations include sodium fluoride, for example, sodium monofluorophosphate (MFP), from about 225 ppm to about 22,500 ppm, acidulated phosphate fluoride (APF) from about 200 ppm to about 12,300 ppm, stannous fluoride from about 900 ppm to about 1500 ppm, for example 960 ppm and 1512 ppm, tin(II) fluoride (SnF2), amine fluorides (e.g., OLAFLUR® (N′-octadecyltrimethylenediamine-N,N,N′-tris(2-ethanol)-dihydrofluoride), DECTAFLUR® (9-octadecenylamine-hydrofluoride)), about 1000 ppm difluorsilane, and calcium fluoride.

The sensitizer solution 12 optionally includes one or more antiperspirants (e.g., aluminum chloride, aluminum chlorhydrate, aluminum zirconium, alum (e.g., crystallized double sulfates of the typical formula M₂SO₄.Mⁱⁱⁱ₂(SO₄)₃.24H₂O, where M is an alkali metal (e.g., lithium, sodium, potassium) and Mⁱⁱⁱ denotes one of the trivalent metals (e.g., aluminum, chromium, or ferric iron), and or perfume fragrances.

In certain embodiments the sensitizer solution 12 contains one or more activation compounds. The activation compounds increase the rate at which other compounds contained in the sensitizer composition (e.g., singlet oxygen, ROS, oxidizers, bleaching agents) undergo chemical reactions. In certain embodiments the activation compounds are catalysts. Examples of activation compounds include macrocyclic metal ligand complexes (such as those disclosed by Collins et al. in U.S. Pat. Nos. 5,853,428, 5,847,120, 6,054,580, 6,099,586, 6,136,223, and 6,241,779, tetraamido macrocycle ligands such as those disclosed by Deline et al. in U.S. Pat. Nos. 6,127,536, 6,297,400 and 6,384,279 and anionic bleaching activators such as those disclosed by Danjo in U.S. Pat. No. 6,797,196, all of which are incorporated herein by reference in their entireties), peroxyacids (e.g., perbenzoic acid), bleach activators, peracid precursors (e.g., esters, ketones, nitrites), transition metal chelates (e.g., those using manganese, copper, and iron), or combinations thereof. Transition metal chelates are used as catalysts for bleaching agents, such as found in U.S. Pat. No. 4,119,557, by Postlethwaite, which discloses the use of iron-polycarboxyamine complexes with hydrogen peroxide releasing substances to clean fabrics. Similarly, U.S. Pat. No. 5,244,594 by Favre et al., U.S. Pat. No. 5,246,621 by Favre et al., U.S. Pat. No. 5,194,416 by Jureller et al., and U.S. Pat. No. 5,314,635 by Hage et al., all of which are incorporated by reference herein in their entireties, describe the use of manganese complexes of nitrogen- (or other heteroatom-) coordinated macrocycles as catalysts for peroxy compounds. Activation compounds can include oxidants. Examples of oxidants include potassium peroxymonosulfate (e.g., Oxone, by DuPont Corp., Wilmington, Del. (CAS 10058-23-8)), complexes of high oxidation state transition metals under the influence of a protein matrix, monooxygenase catalysts, ligands that are resistant to oxidative degradation when coordinated to highly oxidizing metal centers, for example, diamido-N-diphenoxido and diamido-N-alkoxido acyclic chelate compounds and macrocyclic tetraamido-N chelate compounds such as those described by Collins, T. J., “Designing Ligands for Oxidizing Complexes,” Accounts of Chemical Research, 279, Vol. 27, No. 9 (1994), which is incorporated by reference herein in its entirety, macrocyclic tetraamido ligands (e.g., made from azide based synthesis) such as those described by Uffelman, E. S., Ph.D. Thesis, California Institute of Technology, (1992), which is incorporated by reference herein in its entirety, an aryl bridged tetraamido ligand (e.g., synthesized via the azide based route using an aromatic diamine as a starting material), or combinations thereof.

In certain embodiments the transducer 13, such as the acoustic transducer, produces temperature elevation, the formation and/or collapse of microbubbles, and/or rapid expansion (e.g., expansion of a gas contained in microbubbles, boiling: conversion of liquid forms into gaseous forms), and/or cavitation in the sensitizer solution 12 and/or surrounding tissue structures and/or fluids. In certain embodiments the elevated temperature (e.g., through the denaturation of proteins) kills microorganisms. In certain embodiments the elevated temperature in combination with the sensitizer solution 12 kills microorganisms. Elevated temperatures also increase the rate of chemical reactions and the rate of movement of certain compounds into and out of solution, for example oxygen and other dissolved gases, which can increase the effectiveness of certain sensitizer solutions 12. Bubbles, for example, microbubbles are commonly used in conjunction with ultrasound, either external or catheter based, for example, in contrast agents (e.g., OPTISON, AND LEVOVIST, by Molecular Biosystems, Inc., United States). Bubbles contain soluble and/or insoluble components as described herein, for example microspheres (e.g., albumin microspheres containing one or more perfluorocarbons and/or sensitizers). Exposure of microbubbles to ultrasound results in the rapid expansion of the microbubbles and the transmission of mechanical force to the contents of the expanded microbubbles or to the solution components immediately surrounding the expanded microbubbles. In certain embodiments the rapid expansion of sensitizer solution components, for example those contained within or in the immediate vicinity of the microbubbles, results in an increased effectiveness in the penetration and/or delivery of certain sensitizer solution components (e.g., sensitizer) into target organisms and/or tissues. In certain embodiments the cavitation alone, for example through mechanical stress and/or local regions of high temperature, and/or in combination with the effects of the sensitizer solution 12 kills microorganisms. The cavitation produces small bubbles (i.e., cavities) in the sensitizer solution 12, and/or other fluids (e.g., blood, extracellular, and/or intracellular fluids), during the rarefaction half of the wave cycle, followed by the collapse of these bubbles during the compression half of the cycle, as is known to those having ordinary skill in the art. The cavities focus the energy of the incident ultrasonic radiation. The cavities are sites of extremely high temperature (e.g., less than 5000 K to 10⁶ K) and pressure and produce significant mechanical forces such as shear. Cavitation is used in combination with a heat source, for example through the use of an ultrasonic transducer in combination with a heating element (e.g., a resistive conductor with current flowing through it and/or a peltier device), alone and/or in combination with the sensitizer solution 12.

In certain embodiments the sensitizer solution 12 is configured to target, and/or interact closely with, and/or penetrate into microorganisms through various strategies known to one skilled in the art, such as those described by Hasan et al. in U.S. Pat. No. 6,462,070, by Graber et al. in U.S. Pat. No. 6,251,419, and by Wu et al. in U.S. Pat. No. 6,262,030, all of which are incorporated herein by reference in their entireties.

In certain embodiments the sensitizer is coupled, either directly and/or indirectly through a linking molecule, to a compound that targets a specific or limited range of molecules (i.e., a pair-member moiety), for example an antibody. The sensitizer is optionally coupled, either directly and/or indirectly through a linking molecule, to a targeting moiety (e.g., a peptide) other than an antibody or either member of a receptor-ligand pair. (i.e., a non-pair member moiety). The targeting moeity is optionally configured to interact closely or penetrate into a bacteria, virus, fungus or other microorganism. In certain embodiments the targeting moeity increases the cytotoxic effect of the sensitizer, for example, to the target. In certain embodiments the sensitizer is configured to interact closely or penetrate into negatively charged bacteria.

In certain embodiments the targeting moiety includes a polypeptide, for example a linear, branched, or cyclic polypeptide. In certain embodiments the targeting moiety includes a small anti-microbial peptide (SAMP) and or SAMP derivative. Histatins, defensins, cecropins, magainins, Gram-positive bacteriocins, peptide antibiotics, bactericidal/permeability increasing protein (BPI) and combinations thereof. The targeting moiety includes a bacterial, fungal, animal, (e.g., mammalian, such as human), SAMP, an active fragment or analog thereof, or combinations thereof.

The targeting moiety includes a defensin, an active fragment or analog thereof, or combinations thereof. The defensin is: a human defensin (e.g., HNP-1, -2, -3, or -4); a guinea pig defensin (e.g., GPNP); a rabbit defensin (e.g., rabbit NP-1, -2, -3A, -3B, or 5); a rat defensin (e.g., rat NP-1, -2, -3, or -4); murine cryptin; bovine granulocyte bacteriocin or indolicidin; or bovine seminal plasmin.

The targeting moiety optionally includes a SAMP of insect origin, or an active fragment or analog thereof, for example, a cecropin from Cecropia moths, bumble bees, fruit flies, or other insects, an apidaecin from honeybees, or an adropin from fruit flies. The targeting moiety includes a SAMP of amphibial origin, or an active fragment or analog thereof, for example, a magainin, a PGLA, a XPF, a LPF, a CPG, a PGQ, a bombinin, a bombinin-like peptide BLP-1, -2, -3, or -4, or a brevinin. The targeting moiety includes a SAMP from an invertebrate, or an active fragment, or analog thereof, for example, tachyplesin I, II, or III, or polyphemusin I or II, from horseshoe crab. The targeting moiety includes a SAMP of a fish origin (e.g., pardaxin).

The targeting moiety optionally includes a bacteriocin, for example a Gram-positive bacteriocin, or an active fragment, or analog thereof (e.g., a nisin, a subtilin, epidermin, gallidermin, salivarin, a lacticin).

The targeting moiety optionally includes a peptide antibiotic, or an active fragment or analog thereof (e.g., a tyrocidin, or a bacitracin).

The targeting moiety optionally includes a histatin, or an active fragment or analog thereof (e.g., histatin-1 through -8, preferably histatin-1, -3, or -5). The targeting moiety includes histatin-5 residues 13-24, or corresponding residues from other histatins. The targeting moiety includes a histatin molecule that has been engineered to include an internal duplication.

The targeting moiety optionally includes a polypeptide having an affinity for a polysaccharide target (e.g., a lectin). The lectin is a seed, bean, root, bark, seaweed, fungal, bacteria, or invertebrate lectin. The targeting moiety includes a plant polypeptide, e.g., a lectin from jack bean (e.g., concanavalin A, or a lectin from a lentil, Lens culinaris). The targeting moiety includes a salivary polypeptide, or an active fragment or analog thereof. Examples of salivary polypeptides are the histatins (e.g., histatin-1 through -8, or, histatin-1, -3, or -5). The targeting moiety includes histatin-5 residues 13-24, or corresponding residues from other histatins. The targeting moiety includes a histatin molecule that has been engineered to include an internal duplication.

The targeting moiety optionally includes a Gram-negative bacteriocin (e.g., colicin B, colicin E1, or colicin Ia). The targeting moiety includes a bacterially elaborated polypeptide (e.g., nisin, subtilin, epidermin, gallidermin, salivarin, or lacticin).

The targeting moiety optionally includes a molecule (e.g., a peptide) other than an antibody or either member of a receptor-ligand pair. The molecule other than an antibody or either member of a receptor-ligand pair excludes (e.g., it is not coupled covalently or noncovalently) a pair-moiety; an antibody; an enzyme; a hormone; a receptor on a cell surface; or the ligand for a receptor on a cell surface. The targeting moiety includes a peptide in which a single amino ratio of the amino acid residues are of one amino acid residue (e.g., a positively charged amino acid residue), for example, a lysine reside, an arginine residue, an ornithine residue, or combinations thereof. The single amino ratio is more than about 10%, more narrowly more than about 20%, yet more narrowly more than about 30%, yet more narrowly more than about 40%, yet more narrowly more than about 50%, yet more narrowly more than about 60%, yet more narrowly more than about 70%, yet more narrowly more than about 80%, yet more narrowly more than about 90%.

The targeting moiety optionally includes polyamino acids (e.g., polylysine, polyarginine, polyornithine). The targeting moiety is cationic. The targeting moiety has a net positive elementary charge of +1, +2 or +3 per molecule (e.g., a single unit elementary charge is approximately 1.602×10⁻¹⁹ Coulomb). The targeting moiety has a net positive elementary charge equal to or greater than +4. The targeting moiety includes a positively charged amino acid residue (e.g., lysine). The targeting moiety includes at least 2, 3, 4, or more positively charged amino acid residues (e.g., a lysine, arginine, or ornithine residue). The sensitizer is configured to interact closely or penetrate into negatively charged bacteria and/or other microorganisms. The targeting moiety is poly-L-lysine.

The targeting moiety optionally is: anionic. The targeting moiety has a net negative elementary charge of −1, −2 or −3 per molecule. The targeting moiety has a net negative elementary charge equal to or greater than −4. The targeting moiety includes a negatively charged amino acid residue (e.g., aspartic acid, glutamic acid). The targeting moiety includes at least 2, 3, 4, or more negatively charged amino acid residues (e.g., glutamic). The targeting moiety includes at least 10%, 20%, 30%, 40%, or 50% or more negatively charged amino acid residues (e.g., aspartic acid, glutamic acid). The sensitizer is configured to interact closely or penetrate into positively charged bacteria and/or other microorganisms.

The targeting moiety optionally is: approximately neutral in charge. The targeting moiety includes at least 50%, 60%, 70%, 80%, or 90% amino acid residues that are neutral amino acid residues, such as serine, threonine, alanine, methionine, cysteine, or valine.

The targeting moiety has a molecular weight selection from about 1200, 1800, 2400, 3000, 6000, 10,000, 25,000, 50,000, 100,000, or 200,000 daltons or larger. The targeting moiety has a molecular weigh of less than about 250,000, 150,000, 60,000, 25,000, 10,000, 8,000, or 6,000 daltons. The targeting moiety has a molecular weight between about 300 and 1800, 600 and 2400, 1200 and 6,000, 5,000 and 8,000, 8,000 and 15,000, 15,000 and 30,000, 35,000 and 70,000, 70,000 and 150,000, or 150,000 and 300,000 daltons.

The targeting moiety optionally includes a peptide at least 3, 6, 12, 18, 24, 30, 60, 100, 250, 500, 1,000, or 2,500 residues in length. The targeting moiety is a peptide less than 3,000, 1,500, 700, 300, 150, 100, 80, 60, 40, 30, or 15 residues in length. The targeting moiety includes a peptide of between 6 and 15, 12 and 18, 18 and 30, 20 and 40, 30 and 60, 80 and 120, 150 and 300, 300 and 600, 800 and 1,200, or 2,000 and 3,000 residues in length.

The targeting moiety optionally includes a protein that forms a pore in the permeability barrier of the target organism (e.g., in Staphylococcus aureus, Klebsiella pneumoniae, Candida albicans, Leishmania donovani, Giardia lamblia). The targeting moiety is selected using a surface molecule of the target organism as an affinity selection or screen, for example the targeting moiety is selected in a chemical or phage display library.

The targeting moiety optionally includes a low-density lipoprotein, a high-density lipoprotein, a very low-density lipoprotein, or combinations thereof.

The targeting moiety optionally includes a polylysine molecule. The polylysine molecule is between 6 and 15, 12 and 18, 18 and 30, 20 and 40, 30 and 60, 80 and 120, 150 and 300, 300 and 600, 800 and 1,200, or 2,000 and 3,000 residues in length.

The targeting moiety optionally includes a polypeptide (e.g., a polyamino acid) that has been chemically modified to alter its charge (e.g., the charge of side chains of one or more amino acid residues of the polyamino acid). For example, one or more, or approximately 10%, 25%, 50%, 75%, 90% or 100% of the charged side chains is reversed. “Reversed” refers to making a negative side chain (e.g., glutamic acid, aspartic acid), positive or neutral in charge, and/or making a positively charged side chain (e.g., lysine, arginine, ornithine), negative or neutral in charge. For example, one or more of the side chains of polylysine is made neutral or negative in charge.

The conjugate optionally includes a backbone member. The backbone member is coupled to the sensitizer and to the targeting moiety. The backbone member is a targeting moiety, for example polylysine.

In certain embodiments the sensitizer is linked to other molecular fragments and/or particles to increase the residence time, toxicity and/or target specificity of the sensitizer solution 12. Examples of other molecular fragments and or particles include nanoparticles as well as microparticles, polymers, dendrimers, and antibodies such as those described by Chen in U.S. Pat. Nos. 6,344,050 and 6,554,853.

The sensitizer solution 12 optionally includes additives such as buffering agents, acidulants, sequestrants (chelators), nitroxides, antioxidants and/or inert gases. The additives enhance or maintain chemical stability and physiological suitability. Examples of buffering agents include alkali metal hydroxides, carbonates (e.g., sodium carbonate, sodium hydrogen carbonate), sesquicarbonates, borates, silicates, phosphates, imidazole, ammonia, amines, pyridines and other basic aromatic ring compounds, and mixtures thereof. Examples of buffering agents include monosodium phosphate, trisodium phosphate, sodium benzoate, benzoic acid, sodium hydroxide, potassium hydroxide, alkali metal carbonate salts, sodium carbonate, ammonia, pyridine, bipyridine, pyrimidine, pyrazine, imidazole, pyrophosphate salts, citric acid, and sodium citrate. Examples of acidulants include acetic acid, adipic acid, ascorbic, acid, benzoic acid, citric acid, lactic acid, hydrochloric acid, sulfuric acid, carbonic and bicarbonic acid, tartaric acid, malic acid and phosphoric acid; and their corresponding salts such as potassium, sodium, magnesium, calcium and diethanolamine salts. Examples of sequestrants include mono, di and tribasic sodium phosphate, sodium hexametaphosphate, ethylenediaminetatraacetic acid and its alkali metal and alkaline earth metal salts, butyl hydroxyanisol, butyl hydroxytoluene, edetate sodium, edetate disodium, edetate trisodium, edetate calcium disodium, deferoxamine, ditiocarb sodium, aluminum salts, citric acid-sodium salt, gluconic acid-sodium salt, tartaric acid, sodium hexametaphosphate, trientinesodium metaphosphate, sodium pyrophosphate, tetrasodium and tetrapotassium pyrophosphate, sodium tripolyphosphate, polycarboxylic acid and their salts and esters, salts of phosphoric acid and pyrophosphoric acid, citric and tartaric acids. The solution can contain nitroxides, for example, as described by Chang et al. in PCT application WO 2004/105860, by Proctor in U.S. Pat. No. 5,352,442, and by Mitchell et al. in U.S. Pat. No. 5,462,946, all of which are hereby incorporated by reference in their entireties. Nitroxides are stable free radical compounds capable of reacting with a variety of biologically relevant compounds such as free radicals, for example oxy radicals. The nitroxides are free radical scavengers or anti-oxidants. Nitroxides and anti-oxidants ameliorate a portion of negative side effects that result from using photosensitizers and sonosensitizers. The negative side effects include, but are not limited to, oxidative stress, skin phototoxicity, skin sensitivity, and damage caused to healthy cells by the formation of free radicals, including necrosis and apoptosis. Nitroxides prevent subcellular damage including damage to organelles and molecules, such as DNA and RNA. Examples of nitroxides include 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO. Examples of antioxidants, and an exemplary concentration range or example for each antioxidant, include acetone sodium bisulfite from about 0.1% to about 0.8%, ascorbic acid from about 0.05% to about 1.0%, monothioglycerol from about 0.1% to about 1.0%, potassium metabisulfite from about 0.05% to about 0.11%, propyl gallate at about 0.02%, sodium bisulfite from about 0.01% to about 1.0%, sodium formaldehyde sulfoxylate from about 0.03% to about 0.1%, sodium metabisulfite from about 0.02% to about 0.25%, sodium sulfite from about 0.011% to about 0.1%, sodium thioglycolate from about 0.05% to about 0.1%. Other anti-oxidants that are used include, but are not limited to: Vitamins A, B, C, and E, selenium, isoflavones, polyphenols, carotenoids, carnosines, citric acid, phenolic compounds, BHA (butylated hydroxyanisole), BHT (butylated hydroxytoluene), propyl gallate, TBHQ (tert-butyl hydroquinone), lecithins, gum or resin guiac, THBP (trihydroxybutyrophenone), thiodipropionic acid, dilauryl thiodipropionate, co-enzyme Q10, alphalipoic acid, anthocyanins, beta carotene, catechins, ginkgo bilboa, lutien, lycopene, glutathione, and proanthocyanidins.

The inert gas is any gas that, during use with the fluid delivery system, is not reactive. Examples of inert gases include, but are not limited to, molecular nitrogen, carbon dioxide, the noble gases (e.g., helium, neon, argon, krypton, xenon), and combinations thereof.

Certain of the additives, for example acidulants and buffering agents, are used to adjust the pH of the sensitizer solution to be either more basic or more acidic than the treatment site. In certain embodiments the sensitizer solution has a pH from about 3.5 to about 11.5, or about 4, or about 5, or about 6, or about 7, or about 7.14, or about 8, or about 9, or about 10, or about 11. In certain embodiments a sensitizer solution pH of greater than about 7.5 lowers the activation energy required to form free radicals, for example from hydrogen peroxide, thereby increasing the rate of free radical formation.

In certain embodiments the sensitizer solution 12 includes salts, for example the salts of sodium, potassium, chlorine, calcium, magnesium, iron, or combinations thereof. The salts adjust the tonicity of the sensitizer solution 12. The salts can make the sensitizer solution 12 physiologically compatible. The sensitizer composition 12 optionally contains bicarbonate, glucose and/or hydroxyethyl starch.

The sensitizer solution optionally has abrasives. In certain embodiments the abrasives are visible, an example of which is disclosed in U.S. Pat. No. 3,935,306 by Roberts et al., which is incorporated herein by reference. In certain embodiments the abrasives are clear, an example of which is disclosed in U.S. Pat. No. 3,864,470 by Watson. In certain embodiments the sensitizer solution has clear abrasive particles and/or opaque abrasive particles. In certain embodiments the abrasives are transparent and/or transmissive and/or conductive to the energy emissions of the transducers, for example clear abrasives allow the transmission of visible light energy.

In certain embodiments the sensitizer solution 12 includes one or more antimicrobial or preservative agents. In certain embodiments the antimicrobial or preservative agents are in concentrations in the sensitizer solution that provide effective protection from bacteria, yeasts, and/or fungi. The antimicrobial or preservative agents possess anti-microbial inhibitory powers. In certain embodiments the antimicrobial or preservative agents are essentially non-toxic towards humans. The composition and concentration of antimicrobial or preservative agents can depend on the composition of the sensitizer solution, the sensitizer solution's final pH and water activity in the finished formulation. In certain embodiments the antimicrobial and/or preservative function is accomplished by the reactive chemical species contained in the sensitizer solution, for example the sensitizer solution can comprise oxidative reductive potential water solution as described in the incorporated by reference Oculus patent publications and/or the sensitizer solution can comprise solutions with elevated oxygen concentrations, for example those described elsewhere in this application and/or those described in the incorporated by reference TherOx patents and patent publications. In certain embodiments the antimicrobial agent is ethyl alcohol, an acidulant, a sequestrant, a surfactant and/or a flavorant. In certain embodiments the sensitizer solution is preserved by limiting the water available for microbial growth. In certain embodiments the water in the sensitizer solution is limited by replacing it with a humectant such as sorbitol and/or glycerin. The sensitizer solution is evaluated according to known guidelines, (e.g., U.S. Pharmacopeia) to demonstrate that the preservative agent is effective in preventing the multiplication of microorganisms during the shelf life of the product. Antimicrobial and/or preservative agents include sodium benzoate, potassium benzoate, benzoic acid, esters of para-hydroxybenzoic acid (e.g., methylester paraben, ethylester paraben, propylester paraben, butylester paraben, etc.), sorbic acid and its salts, and propionic acid and its salts, boric acid, dioxin (6-acetoxy-2,4-dimethyl-m-dioxane), Bronopol (2-bromo-2-nitropropane-1,3-diol), and salicylanilides (e.g., dibromosalicylanilide, tribromosalicylamilides), CINARYL® 100 and 200 or DOWICIL® 100 and 200 (Cis isomer of 1-(3-chloroallyl-3,5,7-triaza-1-azanidadamantane chloride), hexachlorophene, ethylene diamidetetraacetic acid and its alkali metal and alkaline earth metal salts, phenolic compounds such as chloro- and bromocresols and chloro- and bromo-oxylenols, quaternary ammonium compounds like benzalkonium chloride, aromatic alcohols such as phenylethyl alcohol, benzyl alcohol, chlorobutanol, and quinoline derivatives such as iodochlorhydroxyquinolin, betanapthol, chlorothymol, thymol, anethole, eucalyptol, carvacrol, menthol, phenol, cresol, amylphenol, hexylphenol, heptylphenol, octylphenol, hexylresorcinol, laurylpyridinium chloride, myristylpyridinium chloride, cetylpyTidinium fluoride, cetylpyridinium chloride, cetylpy idinium bromide, phenylmercuric acid, thimerosal, benzethonium chloride, benzalkonium chloride, benzyl alcohol, methyl p-hydroxybenzoate, propyl, p-hydroxybenzoate, and ethylenediaminetetraacetic acid (EDTA) and its alkali metal and alkaline earth metal salts. Examples of antibacterial preservatives, and an exemplary concentration range or example for each antibacterial preservative, include phenylmercuric acid from about 0.002% to about 0.01%, thimerosal at about 0.01%, benzethonium chloride at about 0.01%, benzalkonium chloride at about 0.01%, phenol or cresol at about 0.5%, chlorbutanol at about 0.5%, benzyl alcohol at about 2.0%, methyl p-hydroxybenzoate at about 0.18%, and propyl, p-hydroxybenzoate at about 0.02%.

The sensitizer solution optionally includes peptides. In certain embodiments the peptides are polypeptides, such as linear, branched or cyclic polypeptides. In certain embodiments the peptides are a targeting moiety sensitizer conjugate. In certain embodiments the polypeptides are small anti-microbial peptides (SAMP) and/or SAMP derivatives, histatins, defensins, cecropins, magainins, Gram positive bacteriocins, peptide antibiotics, bactericidal/permeability increasing protein (BPI), enzymes (e.g., those normally found in saliva, e.g., lysozyme, lactoferrin, lactoperoxidase, glucose oxidase), and combinations thereof. The polypeptides include a bacterial, fungal, animal, (e.g., mammalian, such as human) polypeptide, an active fragment or analog thereof, or combinations thereof.

The sensitizer solution 12 optionally includes anticollagenolytic compounds known in the art.

The sensitizer solution 12 optionally includes protection compounds. In certain embodiments the protection compounds protect the composition components from the effects of blood, saliva, sweat, and/or other bodily fluids. Examples of protection compounds include silicon dioxide, fumed silica, silica gels, hydroxyethylcellulose, lanolate, other fatty acids or combinations thereof. Examples of protection agents include those described by Yarborough in U.S. Pat. No. 6,254,388, which is included by reference herein in its entirety.

In certain embodiments protection of the composition components is accomplished by the sensitizer solution, or components therein, acting as a physical barrier, for example, the compositions described in the incorporated by reference TherOx patents and patent publications, for example, the emulsions described in the incorporated by reference TherOx patents and patent publications.

Certain embodiments of the inventions disclosed herein comprise oxygen, and/or other ROS, at partial pressures above ambient levels and the use of these embodiments to treat various medical conditions, such as those disclosed herein and known to those skilled in the art of Hyperbaric Medicine, is contemplated as described herein. In some embodiments of the invention described herein, the use of increased partial pressures of oxygen, and/or other ROS, is important to increase the concentration level of reactive oxygen species, thus, upon use of the embodiment, accelerating the destruction of targeted tissues and/or pathogens, sterilizing and improving any infectious condition and decreasing the time of healing.

The use of oxygen at partial pressures above ambient pressures and concentrations to treat various medical conditions, which include but are not limited to, gas gangrene, necrotizing soft tissue infections caused by aerobic, anaerobic, and mixed bacterial floras viruses and parasites, e.g., progressive bacterial gangrene, necrotizing fascitis, myositis and myonecrosis, infectious diseases, e.g., osteomyelitis, e.g., sternal wound infections, dehiscence, and sternal osteomyelitis, fungal disease, radiation injury, e.g., pelvic radiation necrosis and radiation cystitis, crush injury, compartment syndrome, and other acute traumatic peripheral ischemias, ischemia reperfusion, adjunct therapy to skin grafts and flaps, wound healing, e.g., diabetic ulcers and non or slow healing wounds, treatment of thermal burns, necrotizing chemical exposure, e.g., necrotic arachnidism, is well known and underlies the field of Hyperbaric Medicine and hyperbaric oxygen therapy (HBO). Kindwall E P M.D., Whellan, H T M.D., “Hyperbaric Medicine Practice”, second edition, Best Publishing Company, copyright 1999, ISBN-10: 0941332780, ISBN-13: 978-0941332781, describes the background, history, rational and many uses of hyperbaric oxygen therapy and is incorporated herein by reference in its entirety. A broad review of the role of oxygen tensions in infections is presented in Park M K, Myers R A M, Marzella L. “Oxygen tensions and infections: Modulation of microbial growth, activity of antimicrobial agents, and immunologic responses.” Clin Infect Dis 1992; 14:720-740, which is incorporated herein by reference in its entirety. It is further known that the pharmacological and physiological effects of certain drug products, e.g. antibiotics, chemotherapeutic agents, and the like, can be affected by exposure to partial pressures of oxygen at various levels above those found at ambient conditions, for example, they can be more effective in the killing of targeted tissues e.g., precancerous, cancerous, and the like, and/or pathogens, e.g., microorganisms, and viruses. In certain embodiments the increased partial pressure of ROS, e.g., oxygen, functions to increase the stress, e.g., oxidative stress, that is applied to the targeted pathogens, e.g., microorganisms, viruses and the like, and/or targeted tissues, e.g., precancerous, cancerous, and the like, thereby making them more susceptible to destruction by any applied agents, e.g., antibiotics, antivirals, antimicrobials, antifungals, chemotherapeutic agents, and the like, thus increasing the effectiveness of the agents. In certain embodiments this increase in susceptibility results in a decrease in the amount of applied agent(s), as described herein, required to destroy the targeted tissues and/or pathogens to levels below those normally known in the art—thus reducing the potential side effects related to the applied agents. Applied agents, e.g., antibiotics, chemotherapeutic agents, and the like, known by one skilled in the art to be contraindicated for use with HBO should only be used after carefully considered.

In certain embodiments the significant effects of the inventions herein are localized to the immediate area of application of the inventions, thereby reducing the risk of undesirable systemic side effects. For example, the application of an oxygen enriched emulsion, e.g., those described elsewhere in this application and/or those described in the incorporated by reference TherOx patents and patent publications, comprising, for example, one or more perfluorocarbon compounds, and/or one or more RCS, e.g., ROS, and/or hydrogen peroxide, and/or dilute bleach, and/or oxidative reduction potential water solution as described in the incorporated by reference Oculus patent publications, and any of the other agents as disclosed herein, to a surface wound results only in an increase in the oxygen levels in the tissues in the localized area of application, and not systemically, unlike what is observed during HBO therapy.

In certain embodiments the sensitizer solution 12 includes oxygen. In certain embodiments the sensitizer solution 12 is from about 0.0% to about 100% saturated with oxygen. For example, the sensitizer solution is about 0.0%, or about 10%, or about 20%, or about 30%, or about 40%, or about 50%, or about 60%, or about 70%, or about 80%, or about 90%, or about 100% saturated with oxygen. The sensitizer solution 12 is optionally fully saturated with oxygen. If the sensitizer solution contains oxygen and/or oxygen releasing and/or oxygen generating compounds, the cleaning system delivers oxygen to the treatment site (e.g., fluids and/or tissues and/or structures and/or microorganism and/or surfaces and/or volumes). The oxygen concentration and/or partial pressure of oxygen in the treatment site is then greater than the oxygen concentration and/or partial pressure of oxygen normally seen in the treatment site, for example under conditions of standard atmospheric oxygen concentration, temperature and pressure. In certain embodiments use of the cleaning system increases the partial pressure of oxygen in the immediate and/or local site surrounding the treatment site. In certain embodiments the sensitizer solution comprises oxygen as described in the incorporated by reference TherOx patents and patent publications, for example, in the form of an aqueous solution and/or emulsion, for example a hyperbaric oxygenated aqueous solution and/or hyperbaric oxygenated emulsion.

Sensitizer composition 12 optionally includes ozone. In certain embodiments the sensitizer composition 12 is from about 0.0% to about 100% saturated with ozone. For example, the sensitizer solution is about 0.0%, or about 10%, or about 20%, or about 30%, or about 40%, or about 50%, or about 60%, or about 70%, or about 80%, or about 90%, or about 100% saturated with ozone. In certain embodiments the sensitizer composition 12 is fully saturated with ozone. The sensitizer solution optionally comprises ozone, for example, in the form of an aqueous solution and/or emulsion, for example a hyperbaric ozonated aqueous solution and/or hyperbaric ozonated emulsion. In certain embodiments the hyperbaric solutions are those described in the incorporated by reference TherOx patents and patent publications.

In certain embodiments the sensitizer composition 12 has a gas and/or mixture of gases other than oxygen and/or ozone. Examples of other gases include air, molecular nitrogen, carbon dioxide the noble gases (e.g., helium, neon, argon, krypton, xenon), and combinations thereof. In certain embodiments the sensitizer solution 12 is from about 0.0% to about 100% saturated with one or more gases other than oxygen and/or ozone. The sensitizer solution 12 is, for example, about 0.0%, or about 10%, or about 20%, or about 30%, or about 40%, or about 50%, or about 60%, or about 70%, or about 80%, or about 90%, or about 100% saturated with gases other than oxygen and/or ozone. In certain embodiments the temperature at which the sensitizer solution is manufactured and/or stored and/or used is adjusted to adjust the concentration of the gas in the sensitizer solution.

In certain embodiments the sensitizer solution 12 contains a gas solubility-increasing compound. The gas solubility-increasing compound increases the amount of gas and/or mixture of gases (e.g., air, oxygen, ozone, molecular nitrogen, carbon dioxide, helium, neon, argon, krypton and xenon) that can dissolve in the sensitizer solution 12. In certain embodiments the gas solubility-increasing compound includes perfluorocarbons and/or their derivatives, and/or hemoglobin, and/or modified hemoglobin compounds, for example, pegylated hemoglobin, or mixtures thereof. Examples of gas solubility-increasing compounds include perfluoromethylenes, perfluoroethylenes, perfluorobutanes, perfluoropentanes, perfluorohexanes, perfluorooctanes, perfluorodecalins (PFDs), perfluorohexane, perfluorooctane, octafluoropropane, perfluoroethylcyclohexane, perfluoroindan, perfluoromethylcyclohexane, perfluorodimethylcyclohexane, perfluorotrimethylcyclohexane, perfluorotetramethylcyclohexane, perfluoromethylcyclohexylpiperidine, perfluoromethylethylcyclohexane, perfluorodimethylethylcyclohexane, perfluorotrimethylethylcyclohexane, perfluoromethylcyclopentane, perfluoroperhydrophenanthrene, perfluorodimethylethylcyclohexane, perfluoroperhydrobenzyltetralin, perfluorophenanthrene, perfluoromethyldecalin, perfluorodimethyldecalin, perfluorodiethyldecalin, perfluoromethyladamantane, perfluorodimethyladamantane, perfluoro-6,7H-undec-6-ene, hemoglobin of human origin, hemoglobin of animal origin (e.g., bovine, ovine, porcine, equine, avian), hemoglobin of any origin conjugated to a larger molecule (e.g., polyethylene glycol, piridoxal-5-phosphate, Di-acytyl bis fumerate cross linked hemoglobin, one or more sugars and/or one or more amino acids). The sensitizer solution 12 includes a perfluorocarbon-containing compound, for example, FLUOSOL® DA (Green Cross Corporation, Japan), perflubron or perflubron emulsion (e.g., LiquiVent™ or Oxygent™ both from Alliance Pharmaceutical Corp., San Diego, Calif.), substantially pure straight-chain perfluorocarbon (e.g., Perfluoron® from Alcon, Fort Worth, Tex.), perfluorooctylbromide (e.g., Perflubron), perfluorodichlorooctance (e.g., Oxyfluor®, a 40% v/v solution from HemaGen/PFC, Inc., St. Louis, Mo.), or combinations thereof. Examples of perfluorocarbons and derivatives are those described in the product catalogs of F2 Chemicals Ltd., Lea Lane, Lea Town, Nr Preston Lancashire PR4 0RZ (UK), all of which are incorporated herein by reference in their entireties. In certain embodiments the gas solubility-increasing compound includes modified hemoglobin compounds (e.g., pegylated hemoglobin, that can include polyethyleneglycol (PEG)), peroxides (e.g., hydrogen peroxide, carbomile peroxide), other blood substitutes, ethanol, phenol, and combinations thereof. The gas solubility-increasing compound is a liquid under the conditions of standard ambient temperature and pressure (SATP), 25° C. and 100 kPa. In certain embodiments the gas solubility-increasing compound is a gas under the conditions of SATP. In certain embodiments the gas solubility-increasing compound has a vapor pressure of about 1 mmHg to about 200 mmHg, more narrowly from about 5 mmHg to about 100 mmHg, yet more narrowly from about 30 mmHg to about 50 mmHg, for example about 40 mmHg. In certain embodiments the sensitizer solution comprises solutions as described in the incorporated by reference TherOx patents and patent publications, for example, in the form of aqueous solutions and/or emulsions, for example hyperbaric oxygenated aqueous solutions and/or hyperbaric oxygenated emulsions.

Sensitizer solution 12 optionally contains one or more emulsifiers (i.e., surfactants). In certain embodiments the emulsifiers lower the surface tension of the sensitizer solution, allowing easier spreading, and lower the interfacial tension between components in the sensitizer solution. In certain embodiments the sensitizer solution 12 includes a volumetric emulsifier concentration. In certain embodiments the emulsifier concentration is present in from about 0% to about 30%. In certain embodiments the emulsifier concentration is about 0%, or about 0.001%, or about 0.01%, or about 0.03%, or about 0.05%, or about 0.07%, or about 0.1%, or about 0.3%, or about 0.5%, or about 0.7%, or about 1%, or about 3%, or about 5%, or about 7%, or about 10%, or about 15%, or about 20%, or about 25%, or about 30%. In certain embodiments the surfactant is anionic, nonionic, amphoteric, zwitterionic, cationic, or combinations thereof. Examples of the surfactants are described in Remington's Practice of Pharmacy by Martin and Cook, 12th edition, 1961, pages 219-226, Cosmetics; Their Principles and Practices by R. G. Harry, 1965, pages 396-398 and 413-417, and Cosmetics Science and Technology by E. Sagarin, 1957, pages 328-333, 1060-1063 and 1254, which are herein incorporated by reference in their entirety. Anionic surfactants include sodium, potassium and ammonium soaps derived from fatty acids having from 10 to 22 carbon atoms; and polyvalent metal (magnesium, calcium, zinc, aluminum and lead) soaps derived from fatty acids having from 10 to 22 carbons. In certain embodiments the surfactant is an amine soap derived from fatty acids having from 10 to 22 carbons and primary, secondary and tertiary amines such as monoethanolamine, diethanolamine and triethanolamine, and cyclic amines such as morpholine (e.g., triethanolamine stearate). In certain embodiments the surfactant is a rosin soap such as sodium salts of rosin acids such as abietic acid. In certain embodiments the surfactant is an alkali metal salt of sulfate compound that is represented by the formula ROSO₃H wherein the R group represents an organic moiety such as a fatty alcohol having up to 22 carbons (e.g., sodium lauryl sulfate, sodium cetyl sulfate, sodium monolauryl glyceryl sulfate, an oil such as sulfated castor, olive, teaseed, neat's foot cottonseed, rape seed, corn and rice). In certain embodiments the surfactant is an alkali metal salt of sulfonated compounds that is represented by the formula RSO₃H wherein the R group has from 8 to 22 carbons. Alkali metal salts include alkane sulfonates such as dioctyl sodium sulfosuccinate, oxyethylated alkylaryl sulfate; and/or alkyl aromatic sulfonates such as sodium isopropylnaphthalenesulfonate, sodium dodecylbenzenesulfonate, sodium sulfonaphthylstearate. In certain embodiments the surfactant includes a water-soluble salt of alkyl sulfates having from 8 to 20 carbon atoms in the alkyl radical (e.g., sodium alkyl sulfate) and/or the water-soluble salts of sulfonated monoglycerides of fatty acids having from 8 to 22 carbon atoms. Sodium lauryl sulfate and sodium coconut monoglyceride sulfonates are examples of water-soluble alkyl sulfate salt anionic surfactants. The anionic surfactants include sarcosinates, such as sodium lauroyl sarcosinate, taurates, sodium lauryl sulfoacetate, sodium lauroyl isethionate, sodium laureth carboxylate, and sodium dodecyl benzenesulfonate. In certain embodiments mixtures of anionic surfactants are also used. Nonionic surfactants are broadly defined as compounds produced by the condensation of alkylene oxide groups (hydrophilic in nature) with an organic hydrophobic compound, which is aliphatic or alkyl-aromatic in nature. Examples of nonionic surfactants include poloxamers (e.g., Pluronic and Pluronic R surfactants, for example Pluronic F-68 by BASF Corporation, Florham Park, N.J.), polyoxyethylene, polyoxyethylene sorbitan esters (e.g., TWEENS, for example TWEEN 20 (Polyoxyethylene (20) sorbitan monolaurate) by Cayman Chemical Company, Ann Arbor, Mich.), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures thereof.

Amphoteric surfactants include derivatives of aliphatic secondary and tertiary amines in which the aliphatic radical is a straight chain or branched ad wherein one of the aliphatic substituents contains from about 8 to about 18 carbon atoms and one contains an anionic water-solubilizing group (e.g., carboxylate, sulfonate, sulfate, phosphate, or phosphonate). Amphoteric surfactants are betaines, for example cocamidopropyl betaine. In certain embodiments mixtures of amphoteric surfactants are used.

Cationic agents include amine salts (e.g., hydrochlorides and acetates) derived from straight chain fatty amines having from 8 to 18 carbons, e.g., octodecylamine hydrochloride. Cationic agents include quaternary ammonium salts formed by alkylation of fatty amines with methyl chloride, dimethylsulfate, benzylchloride and the like. The cationic agents are represented by the formula [RR′R″R′″N]Y wherein each of R, R′, R″, R′″ is a long chain aliphatic group of from 8 to 22 carbons or a fatty acid amide; short aliphatic group such as methyl, ethyl, or propyl, an aromatic group such as a phenyl or benzyl radical; or a heterocyclic group such as pyridine or piperidine; and Y represents an inorganic or lower organic ion such as chloride, bromide or acetate radical (e.g., triethanolamine stearate, cetyl trimethyl ammonium bromide, benzalkoniumchloride).

In certain embodiments the emulsifier includes a bile salt, a phospholipid (e.g., egg yolk phospholipid), lecithin, a cross-linked copolymer of acrylic acid and a hydrophobic comonomer (e.g., Pemulen®-TR-1, or Pemulen®-TR-2 by Noveon, Inc., Cleveland, Ohio), a perfluorocarbon ether, or combinations thereof. In certain embodiments the emulsifier includes an emulsifying agent similar to the primary gas solubility-increasing compound, for example a perfluorocarbon, (e.g., Perflubron (i.e., perfluorooctyl bromide)). In certain embodiments the emulsifier is mixed with an emulsifying agent, for example perfluorodecyl bromide. In certain embodiments the emulsifier, for example in the form of an emulsion, is buffered with egg yolk phospholipids. Additional representative emulsifiers (i.e., surfactants) include sorbitan trioleate, sorbitan tristearate, sorbitan sesquioleate, glycerol monostearate, sorbitan monostearate, sorbitan monopalmitate, sorbitan monolaurate, polyoxyethylene lauryl ether, polyethylene glycol 400 monostearate, triethanolamine oleate, polyoxyethylene glycol 400 monolaurate, polyoxyethylene sorbitan monostearate, polyoxyethylenesorbitan monooleate, polyoxyethylene sorbitan monolaurate, sodium oleate, potassium oleate, lauroyl imidazoline, sodium dodecylbenzene sulfonate, sodium monoglyceride sulfate, sodium alkaralkyl polyglycol sulfate, sodium oleyl taurate, sodium dioctyl sulfosuccinate, lauryl polyglycol, ether, sodium dibutylnapthtalenesulfonate, alkyl phenol polyglycol ether, sorbitan monolaurate polyglycol ether, sulfonated castor oil, tall oil polyglycol ester, alkyl dimethyl benzylammonium chloride alkyl naphthalene pyridinium chloride, cetyl dimethyl ethylammonium bromide, alkyl dimethyl chlorobenzylammonium chloride, dibutyl phenyl phenol sulfonate, ester of colaminoethylformyl methylpyridinium chloride, sulfonated methyl oleylamide, sorbitan monolaurate polyglycol ether, polyglycol oleate, sodium lauryl sulfoacetate, sodium 2-ethylhexanol sulfate, sodium 7-ethyl-2-methylundecanol-4 sulfate, sodium 3,9-diethyltridecanol-6 sulfate, sodium lauryl and myristyl collamide sulfonate and N-(sodium sulfoethyl)oleamide.

Sensitizer solution 12 is optionally contained (e.g., microencapsulated), in whole or in part, in bubbles and/or particles (e.g., alginate beads or agar gel beads, liposomes, niosomes, and/or crystals) and/or other form in which a boundary layer is formed to surround the sensitizer and/or components of the sensitizer solution (e.g., macro, micro, and/or nano scale particles and/or spheres (e.g., microspheres (e.g., albumin microspheres). Such formulations are disclosed in U.S. Pat. Nos. 6,375,985; 6,375,968; 6,319,507; 6,217,908; 5,855,865; 4,572,203, and Microencapsulation: Methods and Industrial Applications in Drugs and the Pharmaceutical Sciences, Vol. 73; S. Benita (Ed.); Marcel Dekker; 1996, all of which are hereby incorporated by reference in their entirety, for example, the sensitizer is optionally contained (e.g., microencapsulated), in whole or in part, in nanospheres and/or microspheres and/or macrospheres. In certain embodiments the microspheres have a diameter that is from about 1 to about 700 microns, for example from about 1 to about 5 microns, or from about 5 to about 8 microns, or less than about 8 microns, or from about 8 to about 10 microns, or from about 10 microns to about 20 microns, or from about 20 microns to about 50 microns, or from about 50 microns to about 100 microns, or from about 100 microns to about 200 microns, or from about 200 microns to about 300 microns, or from about 300 microns to about 400 microns, or from about 400 microns to about 500 microns, or from about 500 microns to about 600 microns, or from about 600 microns to about 700 microns. In certain embodiments mixtures of microspheres of different diameters are used.

In certain embodiments sensitizer solution 12 includes oxygen-releasing compounds. The oxygen-releasing compounds include peroxides and other peroxy compounds (e.g., hydrogen peroxide, carbamide peroxide, calcium carbonate peroxide, sodium carbonate peroxide, sodium perborate (monohydrate or tetrahydrate), sodium percarbonate), and combinations thereof.

In certain embodiments sensitizer solution 12 contains bleaching agents (e.g., carbamide peroxide, hydrogen peroxide, calcium carbonate peroxide, sodium carbonate peroxide, ammonium persulfate, sodium persulfate, potassium persulfate, and/or sodium hypochlorite). The sensitizer solution 12 optionally has a bleaching agent concentration. In certain embodiments the bleaching agent concentration is from about 1% w/v to about 80% w/v, more narrowly from about 3% w/v to about 50% w/v, yet more narrowly from about 10% w/v to about 35% w/v. In certain embodiments the bleaching agent concentration is identical to that described in the incorporated by reference Oculus patent publications.

In certain embodiments sensitizer solution 12 includes a transport-improving compound. The transport-improving compound increases the transport efficiency of the sensitizer solution 12 to and through cells (e.g., bacterial cell walls and/or membranes), cell layers (e.g., dermis, epidermis, endothelium, mesothelium), mucosa (e.g., oral, vaginal, urethral, synovial, respiratory), extracellular material, plaque, microbes, debris, or combinations thereof. In certain embodiments the transport-improving compound is a penetrant. In certain embodiments the sensitizer solution 12 includes penetrating solvents. The penetrating solvents enhance percutaneous penetration of the components of the sensitizer solution 12. Examples of the transport-improving material include proparacaine, dimethyl sulfoxide (DMSO), dimethylacetamide, dimethylformamide, tetrahydrofuran, tetrahydrofurfuryl alcohol, 1-methyl-2-pyrrolidone, diisopropyladipate, diethyltoluamide, polymyxin-B nona-peptide (PBNP), hydrocarbons (e.g., squalene and squalane, acetylated lanolin fractions), propylene glycol, substituted azacycloalkan-2-ones having from 5 to 7 carbons in the cycloalkyl group such as 1-dodecylazacycloheptan-2-one (AZONE) and other azacycloalkan-2-ones such as described by Rajadhyaksha in U.S. Pat. No. 3,989,816, which is incorporated herein by reference in its entirety. Examples of the transport-improving material include N-bis-azocyclopentan-2-onyl alkanes described by Rajadhyaksha in U.S. Pat. No. 3,989,815,1-substituted azacyclopentan-2-ones as described by Rajadhyaksha in U.S. Pat. No. 3,991,203, and water-soluble tertiary amine oxides described by Johnson et al. in U.S. Pat. No. 4,411,893. In certain embodiments the transport-improving function is accomplished by an oxygen solubility increasing compound, for example those described elsewhere herein, and/or in the incorporated by reference TherOx patents and patent publications.

In certain embodiments the sensitizer solution contains agents that disrupt or inhibit bacterial biofilms. U.S. Pat. No. 6,726,898 by Jernberg discloses compositions that can disrupt and inhibit bacterial biofilms. In certain embodiments the sensitizer solution containing bacterial biofilm inhibitor or disruptor agents is locally delivered to a treatment site (e.g., sites in the mouth). In certain embodiments the sensitizer solution includes agents that, for example, inhibit or disrupt the glycocalyx matrix of the bacterial biofilm and/or are antagonists of acylated homoserine lactones. In certain embodiments the agents are furanones or furanone derivatives. The sensitizer solution optionally includes agents that bind with or inhibit bacterial lipopolysaccharide. The agents include histatin and/or histatin analogues and/or Dhvar 4. Bacteria employ a cell-cell signaling mechanism in order to produce biofilms. In certain embodiments sensitizer solution 12 includes agents that are antagonists of acylated homoserine lactones, for example certain furanones. U.S. Pat. Nos. 6,337,347 and 6,455,031 disclose example furanones. In certain embodiments the sensitizer solution comprises agents that inhibit autoinducer-2. In certain embodiments the sensitizer solution comprises one or more agents, for example lactoferrin, to inhibit or disrupt glycocalyx matrices, for example of a bacterial biofilm. In certain embodiments the lactoferrin is iron-saturated. In certain embodiments the sensitizer solution has a gingipain inhibitor, for example DX-9065a. The sensitizer solution optionally has a synthetic histatin analogue. E. J. Helmerhorst, et al, The effects of histatin-derived basic antimicrobial peptides on oral biofilms, J. Dent Res 78: 1245 (1999), which is incorporated herein by reference in its entirety. In certain embodiments the agents that disrupt or inhibit bacterial biofilms are those described in the incorporated by reference Oculus patent publications. In certain embodiments the agents that disrupt or inhibit bacterial biofilms are those described in the incorporated by reference TherOx patents and patent publications.

The sensitizer solution 12 optionally is a gel. The gel, as is known in the art, is made by combining the sensitizer with a solvent and adding a gelling agent thereto. Examples of gelling agents include carboxymethyl cellulose, polyacrylates such as the CARBOPOL® brand line of rheology modifiers, (e.g., carboxypolymethylene, CARBOPOL® 934 and/or 934P from Noveon, Inc., Cleveland, Ohio), cellulosic derivatives (e.g., KLUCEL® (cellulose ethers) by Hercules, Inc., Wilmington, Del.), METHOCEL® (methyl cellulose) Dow Chemical Co., Midland, Mich., Natrosol (hydroxyethyl cellulose), gelatin, gums such as agar, tragacenth, acacia gum, guar gum, and guar derivatives, egg yolk, lecithin, pectin, thixcin, and resins like ethyleneoxide polymers, alginic acid and derivatives thereof, colloidal alumina, colloidal silica, and fumed silica (e.g., CAB-O-SIL® from Cabot Corp., Boston, Mass.). U.S. Pat. No. 5,234,342 by Fischer describes a gel that resists degradation in saliva. In certain embodiments one or more gelling agents results in the gel absorbing water. Water absorbing gels, for example hydrogels, are well known to those skilled in the art and are configured to absorb several 100-fold their own weight in water. The water-absorbing agents include polymers (e.g., sodium polyacrylate, potassium polyacrylate, polyacrylamide, dextran, potassium polyacrylate-co-acrylamide, sodium polyacrylate-poly(ethylene oxide), poly(2-hydroxyethylmethacrylate), poly(2-hydroxypropylmethacrylate, sodium poly(isobutylene-co-maleic acid)). In certain embodiments the polymers of water-absorbing agents are cross-linked. The gels are optionally biodegradable or optionally non-biodegradable. The gel is optionally a one-phase or optionally a multiple-phase system. In certain embodiments the gel is a hydroalcoholic gel. For example, an alcohol such as ethanol can dissolve the sensitizer. The sensitizer/ethanol solution is added to a hydrogel. For example, the sensitizer/ethanol solution is added to a premade hydrogel using a slow moving anchor mixer, which reduces the creation of air bubbles in the hydroalcohol gel. Quantities of thickening agents and/or polymers disclosed above are adjusted to adjust the viscosity of the sensitizer solution. In certain embodiments the gel comprises formulations described in the incorporated by reference Oculus or TherOx patents and patent publications. In certain embodiments the aqueous portion of the gel formulations described and/or referenced herein are replaced, in whole or in part, by oxidative reductive potential water solution as described in the incorporated by reference Oculus patent publications. In certain, embodiments the gelling agents, in whole or in part, are replaced by an emulsion formulation as described in the incorporated by reference TherOx patents and patent publications.

The gel is optionally sprayable. According to one embodiment of preparing a sprayable gel, a suitable polymer is added to water. Upon hydration and development of structure, the thickened polymer/water mixture is added to a sensitizer/solvent solution.

In certain embodiments the sensitizer solution 12 has a gelling agent weight concentration. The gelling agent weight concentration is optionally from about 0.1% to about 40 wt %. The gelling agent concentration is about 0.1%, or about 1%, about 3%, or about 5%, or about 7% or about 10%, or about 15%, or about 20%, or about 30%, or about 40%. The gel contains more or less gelling agent to increase or decrease viscosity of the gel. In certain embodiments the sensitizer solution has other adjuvants, for example, waxes such as beeswax, spermaceti, paraffin waxes, and fatty acids, alcohols and amides having from 10 to 22 carbons.

In certain embodiments the sensitizer composition 12 is configured to release composition components, for example the sensitizer, in a timed-release fashion. U.S. Pat. No. 6,197,331 by Lerner et al, teaches time-release methods that are used herein. Lerner et al. discloses a composition for the timed release of compounds into the oral cavity that is used in certain embodiments herein.

In certain embodiments the biodegradable sensitizer composition (e.g., suspension, gel, paste, solid, microparticles, or combinations thereof) is delivered to the treatment site, for example into and/or around a periodontal pocket or wound. The energy is optionally delivered to substantially activate and/or otherwise aid in the performance and/or distribution of the sensitizer composition. In certain embodiments the delivery of energy degrades and releases components from the composition, for example, in a sustained manner. In certain embodiments the formulation of the sensitizer solution as an emulsion, for example the emulsions described in the incorporated by reference TherOx patents and publications, provides for the timed or sustained release of sensitizer components.

In certain embodiments the composition is left on or in the treatment site for sufficient time to degrade (e.g., in part or completely) and for absorption and/or close association (e.g., bound through an antibody, or non-pair member moiety) of the sensitizer by the target microorganism and/or tissue.

In certain embodiments the biodegradable sensitizer composition (e.g., suspension, gel, paste, solid, microparticles, and/or combinations thereof) is delivered to an applicator (e.g., mouthpiece, flexible applicator, bite block). The applicator is applied to an adjacent site. The adjacent site is adjacent to the treatment site.

The biodegradable composition is optionally designed to release the sensitizer or other components in a sustained manner over a period of between 5 minutes and 72 hours. The sensitizer is released in a sustained manner over a period of about 5 min., or about 10 min., or about 15 min., or about 20 min., or about 30 min., or about 60 min., or about 2 hrs., or about 4 hrs., or about 8 hrs., or about 12 hrs, or about 24 hours, or about 36 hrs., or about 48 hrs., or about 60 hrs., or about 72 hrs.

The sensitizer solution 12 optionally includes additives. These additives include cosolvents, surfactants, bioadhesives, or combinations thereof. The cosolvents and surfactants include glycerin, propylene glycol, polypropylene, sorbitol, polymers of polyethylene glycol or other polyols. The bioadhesives include carboxymethylcellulose, polyacrylic polymers, chitosan and sodium alginate, modified starch with polyacrylic polymers, eudispert hv hydrogels or xerogels, sodium hyaluronate, polymers of polyethylene glycol, hydroxypropylcellulose, carboxyvinyl, or combinations thereof. In certain embodiments the additives are incorporated into the sensitizer solution by, for example, mechanically mixing the additives into a mixture of solvent and a gelling agent. Additional formulations suitable for topical administration, for example gels and ointments, are described by Katz et al. in U.S. Pat. No. 3,592,930 and by Shastri et al. in U.S. Pat. No. 4,017,615.

The sensitizer solution 12 optionally includes one or more colorants and/or flavorants. Examples of flavorants include mint flavorings (e.g., essential oil of peppermint, essential oil of spearmint, essential oil of wintergreen), fruit flavoring (e.g., essential oil of cherry, essential oil of grapefruit, essential oil of lemon, essential oil of lime, essential oil of melon, essential oil of orange, essential oil of tangerine), medicinal flavorings (e.g., thymol), meat flavoring, spice flavorings (e.g., essential oil of ginger root, essential oil of cinnamon, essential oil of nutmeg), vegetable flavorings (e.g., essential oil of carrot, essential oil of spinach), mentha oil, and menthol, or mixtures thereof. Flavor compounds consist chemically of aldehydes, ketones, esters, phenols, acids, and aliphatic, aromatic, and/or other alcohols. Flavors may also be compounded with sweeteners. Many sweeteners are available from both natural and synthetic sources. Examples of natural sweetening agents include monosaccharides, disaccharides and polysaccharides such as xylose, ribose, glucose, mannose, galactose, fructose, dextrose, lactose, sucrose, maltose, brown sugar, cane sugar, powdered sugar, honey, maple sugar, invert sugar, molasses, raw sugar, turbinado sugar, partially hydrolyzed starch or corn syrup solids, and sugar alcohols such as sorbitol, xylitol, mannitol, malitol, robitol, erythritiol, lactitol, and mixtures thereof. Examples of artificial sweeteners include the soluble saccharin salts (i.e., sodium, or calcium saccharin salts), aspartame, sucralose, stevia, cyclamate salts, acesulfame-K, and the free acid form of saccharin.

The sensitizer solution optionally includes colorants including dyes suitable for food, drug and cosmetic applications, known as FD & C dyes. The materials acceptable for the foregoing uses are water-soluble. Colorants include FD & C Blue No. 2 (i.e., disodium salt of 5,5-indigotindisulfonic acid), or FD & C Green No. 1 (i.e., the monosodium salt of 4-[4-N-ethyl-p-sulfobenzyl amino)diphenylmethylene]-[1-(N-ethyl-N-p-sulfoniumbenzyl)-2,5-cyclohexadie nimine]). A full recitation of all FD & C and D & C colorants useful in the present invention and their corresponding chemical structures is found in the Kirk-Othmer Encyclopedia of Chemical Technology, 3rd Edition, in Volume 6, which is incorporated herein in its entirety.

The sensitizer solution 12 and all components thereof are biocompatible for applications in the medical, dental, and related fields. Non-biocompatible solutions are contemplated for other applications.

In certain embodiments the sensitizer solution 12 contains ions and/or compounds and/or particles, hereafter charged particles, that have a net negative or positive charge (e.g., atoms, molecules, crystals, conjugated molecules, complexed molecules, micelles, liquid crystal, liposomes niosomes, caged molecules, and/or particles (e.g., macro, micro and/or nano scale particles and/or spheres and/or crystals)), and can experience a force in the presence of an electric field. An electric field is optionally applied to the sensitizer solution and/or treatment site to induce the motion and/or orientation of charged particles. The motion and/or orientation of the charged particles directly results in the motion and/or orientation of non-charged particle in the solution and/or other fluids in the treatment site due to the properties of the fluids (e.g., viscosity). In certain embodiments the electric field is used to separate molecules, perform electrochemistry, electrophoresis, iontophoresis, electroporation, control liquid crystals, and combinations thereof. The charged particles are optionally connected to the sensitizer. In certain embodiments the sensitizer and/or molecules connected to the sensitizer, for example, targeting moieties, are among those compounds that experience a force in the presence of an electric field.

The sensitizer solution 12 optionally contains magnetic compounds and/or particles, hereafter magnetic particles, that experience a force in the presence of a magnetic field (e.g., atoms, molecules, crystals, conjugated molecules, complexed molecules, micelles, liquid crystals, liposomes, niosomes, caged molecules, macro, micro and/or nano scale particles and/or spheres and/or crystals). Magnetic particles are ferromagnetic, paramagnetic, superparamagnetic and/or diamagnetic compounds and/or particles. This force directly results in the motion and/or orientation of the magnetic particles. The motion and/or orientation of the magnetic particles directly results in the motion and/or orientation of non-magnetic particle in the solution and/or other fluids in the treatment site due to the properties of the fluids (e.g., viscosity). The magnetic particles include those described in U.S. Pat. No. 6,797,380 by Bonitatebus, et al., which describes nanoparticles comprising an inorganic core of, for example, a superparamagnetic material and a ionizable polymerizeable outer coating to which a number of molecule classes are optionally connected. The magnetic particles include monocrystalline iron oxide nanoparticles (MIONs) and/or cross-linked iron oxide nanoparticles (CLIOs). In certain embodiments the magnetic particles are connected to the sensitizer. The sensitizer and/or molecules connected to the sensitizer, for example, targeting moieties, are optionally among those compounds that are affected by the magnetic field.

The sensitizer solution 12 is coupled to and/or enclosed in a dendrimer or dendrimer based structure, for example, those available from Dendritic NanoTechnologies, Inc., Mount Pleasant, Mich. Chowdhary et al. in U.S. Pat. No. 6,693,093 describes the use of block copolymers of the non-toxic di-block, symmetric and non-symmetric tri-block copolymers and dendrimer types to enhance the stability and deliverability of photosensitizers. The dendrimer acts, for example, to modify the solubility of the sensitizer, increase the ability of the sensitizer to be delivered close to the target organism (e.g., through the control of the dendrimers internal and/or surface charge), target the sensitizer to a specific target organism or related group of organisms (e.g., gram negative or gram positive bacteria), and/or increase the ability of the sensitizer to be transported across the membranes and/or cell walls of target organisms.

In certain embodiments the sensitizer is coupled to one or more molecules, forming a conjugate-sensitizer complex, by a bond and/or bonds that are broken by the application of energy, hereafter “cleavage energy,” for example photolabile and/or sonolabile bonds. The cleavage energy is one or more particular frequencies and/or intensities and/or durations and/or repetition rates or ranges of frequencies and/or intensities and/or durations and/or repetition rates. In certain embodiments the conjugate-sensitizer complex is more or less cytotoxic than the sensitizer alone, or optionally the conjugate-sensitizer complex has no cytotoxic effects. In certain embodiments the conjugate-sensitizer complex's cytotoxicity in the presence of sunlight, room lighting or the under application of energy that would activate the sensitizer alone is reduced or eliminated. This reduction and/or elimination of cytotoxicity is a result, for example of the conjugate molecule(s) charge and/or size and/or attachment location on the sensitizer molecule, and/or shape, for example through steric hindrance or by preventing the conjugate-sensitizer complex from entering into or getting close to either the targeted organisms or the cells of the host organism. In certain embodiments the reduction and/or elimination of the cytotoxicity are the result of the conjugate-sensitizer complex being unable to produce ROS, for example singlet oxygen. The choice of a specific protecting group is based on the structure of the sensitizer, sensitizer activation energy frequency, cleavage energy frequency, biological activity of the sensitizer-conjugate complex and of the separated conjugate, solubility of the sensitizer-conjugate complex, rate of deprotection, absorption characteristics of the sensitizer and target site, reactivity of by-products (e.g., it is desirable for the protecting group to be non-toxic, soluble in biological media, and have limited reactivity when separated from the sensitizer), and on the chemical conditions, for example pH, of the sensitizer solution and target site. Examples of photolabile conjugate molecules, and in certain cases an example wavelength for cleavage of the connecting bond, include anthraquinon-2-ylmethoxycarbonyl (350 nm), ortho-nitrobenzyl groups (e.g., nitroveratryloxycarbonyl (320 nm), nitrobenzyl oxycarbonyl (320 nm), di(nitrobenzyl)oxycarbonyl (320 nm), 2-(2-nitrophenyl)propoxycarbonyl (NPPOC) (365 nm)ₘ, ortho-Nitro-benzyl-type (MeNPOC), ortho-Nitrophenyl-ethyl-type (NPPOC), phenacyl groups (e.g., phenacyl (308 nm), α-methylphenacyl (313), 4-methoxyphenacyl (313), 4 hydroxyphenacyl (300)), benzoin esters or desyl compounds (e.g., 3,5-dimethoxybenzoin (366 nm), 3,3′,4,4′ dimethylenedioxybenzoin (366 nm), 2,2′,3,3′ dimethylenedioxybenzoin (366 nm)), 6-bromo-7-hydroxycoumarin-4-ylmethyl, 8-bromo-7-hydroxyquinoline (365 nm), arylazidoalcohols (300 nm), 2-(dimethylamino)-5-nitrophenyl (400 nm), 2,4-dinitrobenzenesulfenyl esters (300 nm), nitroindolines (305 nm), o-nitrophenylethylene glycol (350 nm), Bis o-nitrobenzyl alcohol (350 nm), Bis o-nitrobenzyl ethanediol (350 nm), 1,3-Dithiane (300 nm), 2-phenyl-1,3-dithiane (300 nm), coumarin diol (365 nm), coumarin (366 nm), vinylic phenols (255 nm), vinylic napthols (350 nm), benzyl alcohols (e.g., benzyloxycarbonyl (254 nm), m-dimethoxy Cbz (350 nm), 2-(3,5-dimethoxyphenyl) propyl-2-oxycarbonyl (276 nm or 282 nm), Sulphonamides (e.g., Tosyl (300 nm), 2-aryl-4-quinoline (350 nm), polycyclic aromatic hydrocarbons (e.g., those described in T. Furuta, Y. Hirayama, M. Iwamura, Org. Lett. 2001, 3, 1809, which is incorporated by reference herein in its entirety, for example Aqmoc, Pmoc, Mcmoc, Phmoc), Polysilanes, N-methyl-N-(o-nitro) carbamate (254 nm), and 2-benzoylbenzoic acid (300-390 nm). Further examples of photosensitive protecting groups are described by Pillai, Synthesis 1980, 1; Pillai, Org. Photochem. 1987, 9, 225; Dorman & Prestwich, TIBTECH 2000, 18, 64; Bochet, Perkin 1, 2002, 125 and are incorporated herein by reference in their entirety. In certain embodiments the cleavage energy is different in frequency and/or intensity and/or duration and/or repetition rate than the energy used to activate the sensitizer solution. The cleavage energy is optionally applied before and/or during and/or after delivery of the sensitizer solution. The cleavage energy is optionally applied before and/or during the application of the energy used to activate the sensitizer solution.

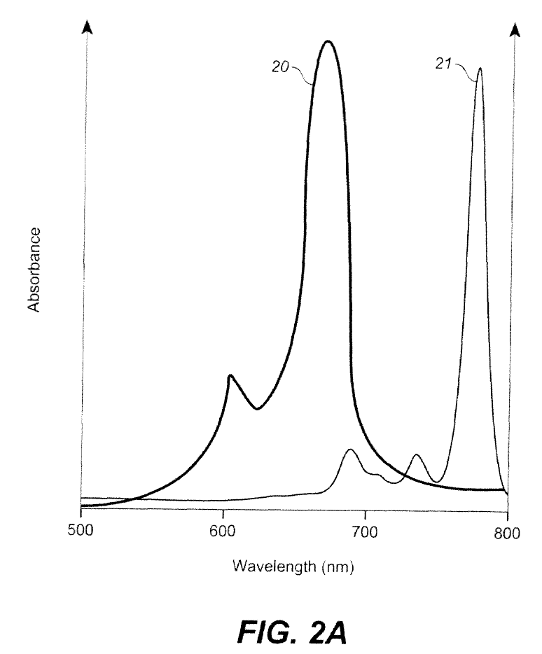

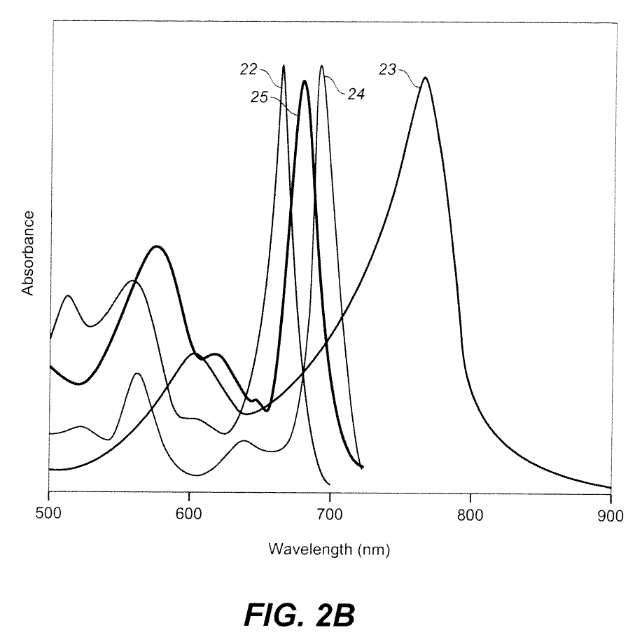

FIGS. 2A and 2B illustrate that the sensitizer solution 12 has an electromagnetic energy absorption level dependent on the frequency (i.e., wavelength) of the electromagnetic energy. In certain embodiments the absorption spectrum of the sensitizer solution 12 is dominated by absorption over one or more narrow frequency ranges or can exhibit a more consistent absorption over a broader frequency range. In certain embodiments the sensitizer solution has St. John's wart, hypericin, erythrosine B, or combinations thereof as photosensitizers that can exhibit absorption over a broad range of visible light frequencies. See U.S. Pat. No. 6,561,808 by Neuberger.

The absorption graph 20, shown in FIG. 2A, illustrates a representative absorption spectrum for a metalated (e.g., zinc, or silicone, or aluminum) phthalocyanine. Metalated, sulfonated, hydroxylated and alkoxylated derivatives exhibit absorption curves similar in character. The absorption graph 20 forms a curve having substantially normal characteristics for the primary peak and has a general range of absorption from about 570-730 nm and a peak range of absorption from about 670-690 nm, with a primary peak at about 670 nm, and a secondary peak at about 605 nm.

The second absorption graph 21, shown in FIG. 2A, illustrates the absorption spectrum for silicon naphthalocyanine. The second absorption graph 21 forms a curve having substantially normal characteristics for the primary peak. The graph has a primary peak at about 773 nm, a secondary peak at about 690 nm, and a tertiary peak at about 735 nm.

A third absorption graph 22, shown in FIG. 2B, illustrates the absorption spectrum for chlorin e₆. The third absorption graph 22 forms a curve having substantially normal characteristics for the primary peak. The graph has a primary peak at about 665 nm, a secondary peak at about 575 nm, and a tertiary peak at about 515 nm.

A fourth absorption graph 23, shown in FIG. 2B, illustrates the absorption spectrum for bacteriochlorin a. The fourth absorption graph 23 forms a curve having substantially normal characteristics for the primary peak. The graph has a primary peak at about 765 nm, and a secondary peak at about 605 nm.

A fifth absorption graph 24, shown in FIG. 2b, illustrates the absorption spectrum for the purpurin NT₂H₂. The fifth absorption graph 24 forms a curve having substantially normal characteristics for the primary peak. The graph has a primary peak at about 690 nm, a secondary peak at about 563 nm, and a tertiary peak at about 638 nm.

A sixth absorption graph 25, shown in FIG. 2b, illustrates the absorption spectrum for A- and B-ring benzoporphyrin derivatives. The sixth absorption graph 25 forms a curve having substantially normal characteristics for the primary peak. The graph has a primary peak at about 680 nm, a secondary peak at about 575 nm, and a tertiary peak at about 618 nm.

In certain embodiments sensitizer solution 12 has a peak absorption wavelength or frequency about equal to the peak emission wavelength or frequency of the transducer 13. In certain embodiments the sensitizer solution 12 has a peak absorption range in the visible spectrum, for example from about 400 nm to about 700 nm.

In certain embodiments sensitizer solution 12 has a peak absorption wavelength or frequency at the red end of the visible spectrum or longer, for example the peak absorption wavelength is in the red and/or far red and/or near infrared range, from about 625 nm to 1400 nm. More narrowly the sensitizer solution 12 has a peak absorption wavelength of from about 700 nm to about 1000 nm. Light in this frequency range can penetrate tissues, for example oral tissues, dermal tissues and blood, which may be present in the treatment site, more effectively than some other frequencies of visible light, thereby more effectively activating the sensitizer solution after passing through such tissues. In certain embodiments sensitizers, for example with a peak absorption above about 700 nm, are also colorless. The sensitizer solution, for example colorless sensitizer solution, causes little or no staining or discoloration of the surfaces being treated.

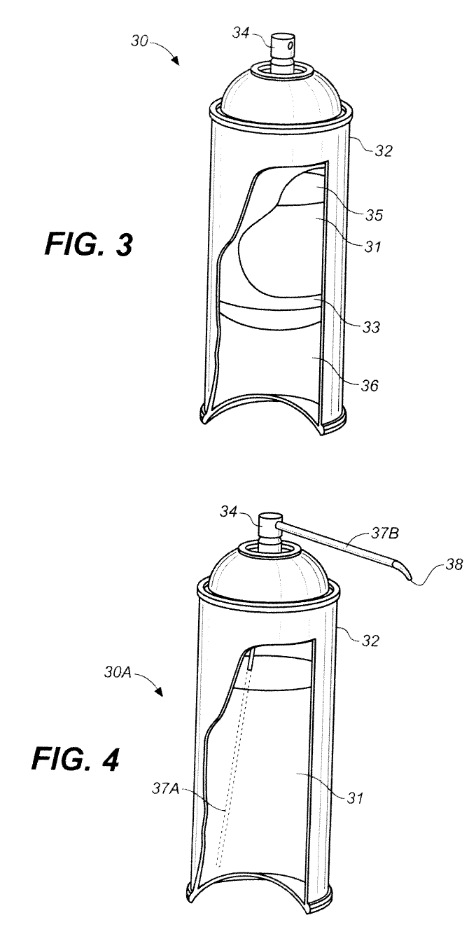

FIG. 3 illustrates that the fluid delivery system 30 has a sensitizer solution 31, for example within the cartridge 32. The cartridge 32 contains a sealed container, for example a bladder 33. The cartridge 32 is sensitizer solution-tight and/or airtight and is rigid or flexible.

In certain embodiments the bladder 33 is a rigid (i.e., non-compliant), or flexible (i.e., compliant), or semi-compliant container, for example an elastomeric (e.g. silicone, silicone RTV, latex, vulcanized rubber, buna rubber, VITON®, neoprene, fluorosilicone rubber. EPDM rubber, nitrile rubber, polyurethane, SANTOPRENE®), and/or polymeric (e.g., polyethylene (LDPE, LLDPE, HDPE), polypropylene, polyvinylchloride (PVC), polystyrene, nylon, polyester, mylar), and/or metal foil, and/or metallized polymeric and/or elastomeric bag 33. In certain embodiments the bladder 33 is frangible and/or breakable, for example made of glass, ceramic, or brittle polymer. The bladder 33 is optionally constructed from one or more layers. The bladder 33 can hold one or more sensitizer solutions and zero, one, or more than one pressurized gases. The bladder 33 is optionally gas impermeable. The bladder 33 is made from, for example as one of the layers of the bladder 33, a metal foil. The bladder 33 optionally has a bladder coating. In certain embodiments the bladder coating is on the inside and/or outside of the bladder 33. The bladder coating is optionally a gas impermeable coating, for example Parylene (polypara-xylylene) (e.g., Parylene N, Parylene C, and/or Parylene D). The bladder coating can prevent the remainder of the bladder from contacting the contents of the bladder and/or the cavity. The bladder 33 can prevent the sensitizer solution 12 from contacting the contents of the cavity and/or the surface of the cartridge 32.

A bladder valve 34 is in fluid communication with the bladder 33 and the outside of the cartridge 32. The bladder valve is part of and/or the same as the valve 34. The bladder valve is configured to controllably release the sensitizer solution 12 from the bladder 33.

The fluid delivery system 30 optionally has an intra-bladder gas volume 35. The intra-bladder gas volume 35 occupies about 0 to 80% of the bladder 33 volume, for example about 0%, or about 10%, or about 20%, or about 30%, or about 40%, or about 50%, or about 60%, or about 70%, or about 80% of the bladder volume is intra-bladder gas volume 35. The intra-bladder gas volume 35 optionally contains a gas (e.g., oxygen, ozone, or an inert gas) and/or mixture of gases (e.g., oxygen, and/or ozone, and/or an inert gas). The gas optionally has a pressure of about 0 psig to about 3500 psig, for example about 125 psig, or about 250 psig.

In certain embodiments the fluid delivery system 30 has a cavity 36. The cavity 36 occupies about 0 to 80% of the cartridge 32 volume, for example about 0%, or about 10%, or about 20%, or about 30%, or about 40%, or about 50%, or about 60%, or about 70%, or about 80% of the volume of the cartridge 32 is cavity volume. The cavity volume 36 optionally contains a gas (e.g., oxygen, ozone, or an inert gas) and/or mixture of gases (e.g., oxygen, and/or ozone, and/or an inert gas). The gas has a pressure of about 0 psig to about 3500 psig, for example about 125 psig.

In certain embodiments the intra-bladder gas volume 35 is about 0% of the internal bladder 33 volume and substantially all of the internal bladder volume 35 is filled with the sensitizer composition. The extra-bladder volume 35 is pressurized with a gas, which acts to propel the sensitizer composition 31 from the delivery system 30 when the valve 34 is actuated. In other embodiments the bladder 33 substantially fills the complete volume of the fluid container 32 leaving no appreciable cavity volume 36. In these embodiments the internal volume of the bladder 33 is filled by the sensitizer solution 31 and a volume of pressurized gas occupying the intra-bladder gas volume 35. A pressurized gas or mixture of gases occupies the intra-bladder gas volume 35 and the extra-bladder volume (or cavity) 36. The gas or mixture of gases occupying these volumes is the same gas or different gases. The intra-bladder gas volume 35 optionally contains oxygen, for example 100% oxygen, and the extra-bladder volume 36 optionally contains an inert gas, for example nitrogen.

In a specific embodiment the bladder 33 is flexible, for example silicone and has a gas impermeable layer, for example Parylene, coating the inside and/or outside. The cartridge is a standard steel aerosol can, well known to those skilled in the art. The bladder 33 fills about 60% of the volume of the cartridge 32. The cavity 36 is filled with an inert gas, for example nitrogen gas, to a pressure of about 125 psi. About 10% of the intra-bladder volume is filled with pure oxygen at a pressure of about 125 psig and the remainder filled with a flowable aqueous sensitizer solution 31. The flowable aqueous sensitizer solution 31 has Toluidine Blue O, a metalated naphthalocyanine, for example Zn, Si, or Al, a metalated phthalocyanine, for example Zn, Si, or Al, or combinations thereof. The flowable aqueous sensitizer solution has a perfluorocarbon, for example perfluorodecalin. The flowable aqueous sensitizer solution 31 is at a temperature of about 21° C. (70° F.).

Valve 34 is in fluid communication with the bladder 33 and the environment outside of the cartridge 32. The valve 34 is configured to controllably release the sensitizer solution 31 from the bladder 33 to the environment outside of the cartridge 32.

Valve 34 has a fluid control, for example a spray nozzle. The fluid control is configured to increase the velocity and/or aerate the sensitizer solution 31 exiting the cartridge 32. For example, when the spray nozzle is depressed, the valve opens and releases the sensitizer solution 31. In certain embodiments, bladder 33 contains one or more delivery conduits similar to those indicated in FIG. 4.

FIG. 4 illustrates that the cartridge 32 is pressurized with a propellant gas and is absent of the bladder 33. The propellant gas pressurizes the sensitizer solution 31 in the cartridge 32. In certain embodiments the propellant gas is inert, while in other embodiments it provides functional enhancement of the solution, for example, the propellant gas is optionally oxygen or high in oxygen concentration. Due to the increased pressure in the cartridge 32, the oxygen concentration in the sensitizer solution 31 is increased relative to the oxygen concentration of the solution at atmospheric conditions. This increased level of oxygen is available for the formation of singlet oxygen and/or other ROS.

Cartridge 32 optionally contains one or more containers (not shown), for example ampoules, that are sealed to prevent the contents of the ampoule and the sensitizer solution from mixing until the desired time. U.S. Pat. Nos. 4,893,730, 4,941,615, 4,979,638, 5,012,978, 5,018,643, and 5,154,320 by Bolduc, describe such systems. Separation of the contents of the ampoule and the sensitizer solution increases the shelf life and/or stability of the ampoule contents and/or the sensitizer solution. The ampoule is frangible or breakable. In certain embodiments the contents of the ampoule are pressurized. The ampoules contain compounds that when released mix with the sensitizer solution but do not undergo a chemical reaction. The ampoules contain compounds that when released undergo one or more chemical reactions with the sensitizer solution. The chemical reactions, for example, result in the production of RCS, ROS, ROS precursors, oxygen, photosensitizers, and/or photosensitizer precursors. The chemical reactions result in the pressure inside the cartridge being increased.

One or more delivery conduits 37A, 37B are attached to the valve 34. The delivery conduits 37A, 37B are tubes, channels, open, closed, or combinations thereof and are rigid, flexible, hinged, otherwise articulatable or combinations thereof. The delivery conduits 37A, 37B are opaque, translucent, or transparent to the frequency of electromagnetic energy emitted by the transducer (not shown). The delivery conduits 37A are substantially straight, curved, coiled, or combinations thereof. The delivery conduits 37A can extend away from the valve 34 (e.g., from a spray nozzle) into and/or out of the cartridge 32.

The delivery conduit 37B has one or more fluid outlets 38. In certain embodiments the fluid outlet 38 is at the end of the delivery conduit 37B farthest from the cartridge 32. Fluid outlets 38 perforate lengths of the delivery conduit 37B.

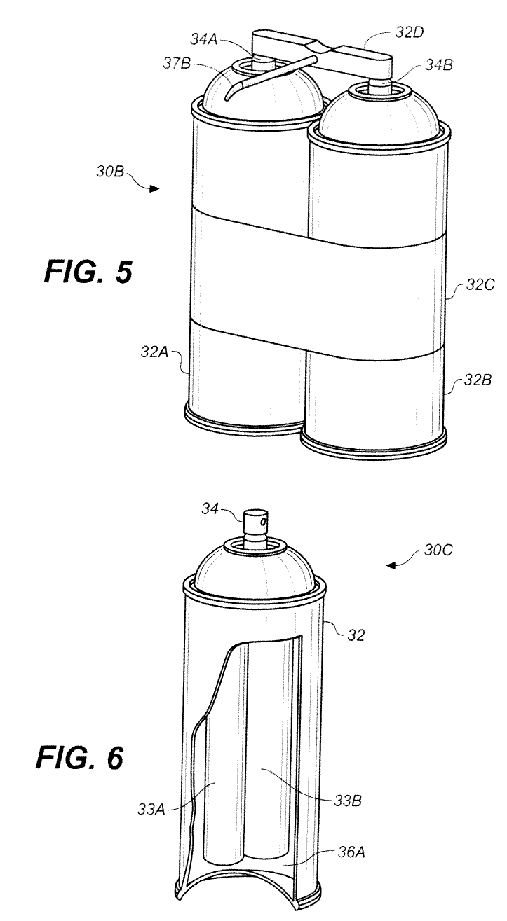

FIG. 5 illustrates that the cleaning system 30B has first and second cartridges 32A and 32B. More than two cartridges 32A and 32B are used. The first cartridge 32A is removably or fixedly attached to the second cartridge 32B, for example, with an adhesive, a band 32C (as shown), interlocking configurations, or combinations thereof.

The first and second cartridge 32A and 32B are in fluid communication with a head, for example a joining cap 32D. The joining cap 32D is integral with or removably attached to the delivery conduit 37B. The joining cap 32D is configured to controllably route flow out of the cartridges 32A and 32B into and through the delivery conduit 37B. The first cartridge 32A has a first valve 34A. The second cartridge 32B has a second valve 34B. The joining cap 32D is removably or fixedly attached to the first valve 34A and/or the second valve 34B.

By controllably routing or using other designs or methods, the joining cap 32D is configured to mix the contents of the first and second cartridges 32A, and/or 32B, and/or external environment (e.g., air) in a ratio automatically controlled or manually controlled by one or more valves (not shown) on the joining cap 32D that includes one or more knobs, switches, dials, levers, toggles, tabs, buttons, slides, accelerometers, fluid or contact pressure sensors, other rotating switches, other translating switches, or combinations thereof.

The first and second cartridges 32A and 32B optionally have different contents. For example, the first cartridge 32A is substantially filled with the sensitizer solution 31, and the second container 32B is substantially filled with oxygen, an oxygen saturated solution, and/or solutions containing one or more compounds that release oxygen, for example, upon mixing with the first solution or upon contact with saliva and/or the oral mucosa. Both cartridges 32A and 32B are optionally substantially filled with the sensitizer solution 31.

FIG. 6 illustrates that the cartridge 32 has a first bladder 33A and a second bladder 33B. The bladders 33A and 33B are individual cartridges within the cartridge 32. The first and second bladders 33A and 33B are in fluid communication with the valve 34. The valve 34 is configured to mix the contents of the first bladder 33A, and/or the second bladder 33B, and/or the outside environment (e.g., air), for example when the valve 34 is activated. The valve 34 is configured to manually and/or automatically control the ratio of the contents of the first bladder 33A, and/or the second bladder 33B, and/or the outside environment in any fluid dispensed through the valve 34, as described above for the joining cap 32D. In certain embodiments one or more of the bladders 33A and/or 33B contain one or more delivery conduits similar to those indicated in FIG. 4.

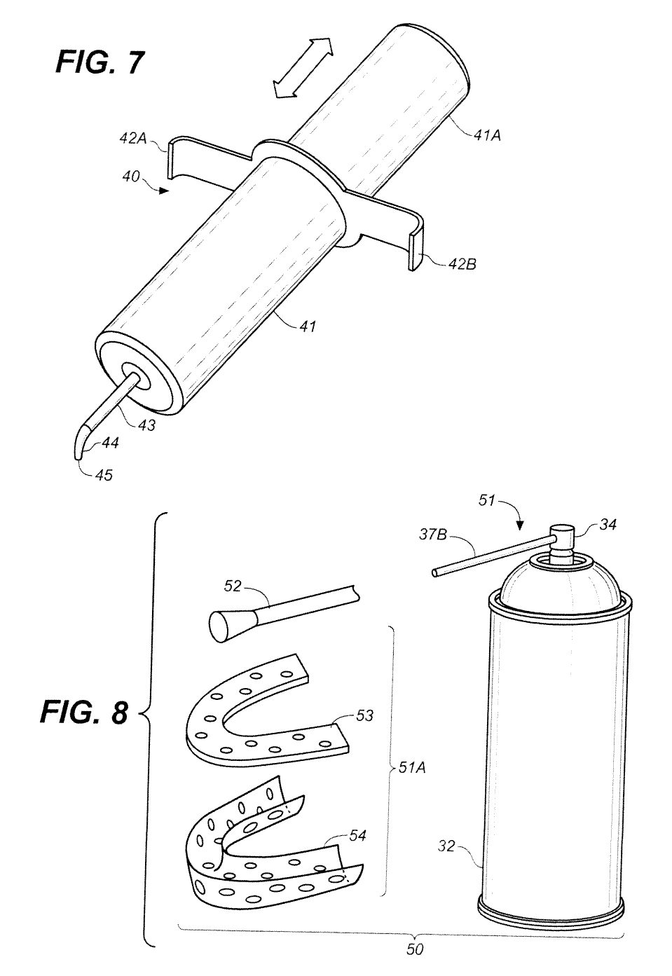

FIG. 7 illustrates that the cartridge 41A is optionally slidably attached, as shown by arrows, to the head 41. Head 41 has grips 42A, 42B that are ergonomically configured to be held with the fingers and/or hand. The delivery conduit 43 is integral with the head 41 and/or the valve in the cartridge 41A. The cartridge 41A is slidably attached, or not attached, to the head 41.

The valve in the cartridge (not shown) is designed to controllably release the sensitizer solution. The cartridge 41A is releasably or fixedly attached to the head 41, for example, with or to a cartridge connector (not shown) on the head 41. In certain embodiments the cartridge 41A has a modular seal, for example a seal that is opened and closed by the cartridge connector on the head. In certain embodiments the valve in the cartridge 41A has a breakable seal (not shown). The delivery conduit 43 has an inlet (not shown). The inlet and/or a component of the valve is configured to open (e.g., break or puncture) the breakable seal (not shown) and activate the valve. When the breakable seal is opened and the valve actuated, the contents of the cartridge 41A, such as the sensitizer solution flow through the delivery conduit 43 and out the fluid outlet 45. When the inlet is removed from the breakable seal, the breakable seal is configured to close and reseal or remain open. The cartridge 41A is optionally spring-loaded in the head 41, for example, such that the breakable seal is not opened by the inlet unless an external force is applied to press the fluid container 32 into the head 41.

The delivery conduit 43 has a neck 44. The neck forms a sharp or smooth angle with the remainder of the delivery conduit 43. The neck 44 is completely or substantially straight, curved, angled, coiled, fixed, articulatable, or combinations thereof. The neck is flexible or rigid. The delivery conduit 43 is transparent or translucent. The neck 44 is transparent or translucent. In certain embodiments an electromagnetic energy source in or on the head 41 or delivery conduit 43 or cartridge 32A transmits electromagnetic energy into the delivery conduit 43. The delivery conduit 43 transmits the electromagnetic energy into the neck 44. The neck 44 transmits the electromagnetic energy into the treatment site.

FIG. 8 illustrates that the cleaning system 50 has a separate fluid delivery system 51 and one or more separate applicators 51A (e.g., wand 52, wafer 53, mouthpiece (i.e., dental tray) 54), or combinations thereof. One or more transducers (e.g., illuminating devices) are optionally on, in or otherwise attached to the applicators. The illuminating devices are used in conjunction or replaced by a vibrating device, for example during use of a sonosensitizer solution.

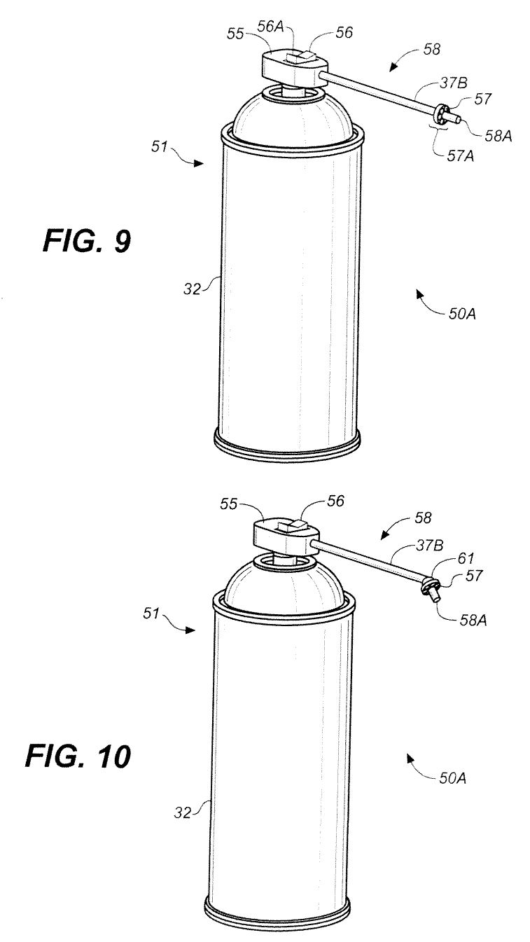

FIG. 9 illustrates that the cartridge 32 is integral with or fixedly or removably attached to the head 55. The head 55 and/or cartridge 32, and/or delivery conduit 37B have one or more transducers 57A (e.g., illuminating devices 57A), ultrasonic transducers, electric and/or magnetic field sources) (not shown) and/or one or more power cells, for example batteries. The transducers are in or on the head 55 and/or cartridge 32, and/or delivery conduit 37B. The transducers have the same or differing energy emission profiles. The head 55 has one or more delivery conduits 37B, and/or light controls 56. The light controls are configured to control any adjustable characteristic of the energy (i.e., not necessarily light) emitted by the transducers. In certain embodiments the light control 56, as shown, is replaced by the fluid control, and/or one or more fluid controls and light controls 56. The fluid controls and/or light controls 56 are knobs, switches, dials, levers, toggles, tabs, buttons, slides, accelerometers, fluid or contact pressure sensors, other rotating switches, other translating switches, or combinations thereof. The fluid controls and/or light controls 56 are on the head 55, the cartridge 32, elsewhere (e.g., an external control pad), or combinations thereof. In certain embodiments the light control 56 and the fluid control 56A are the same control mechanism. For example, one control mechanism activates and/or adjusts the fluid flow quantity and the light intensity. The light controls 55 activate and/or adjust the energy emission profile of the illuminating device 57. Energy from the transducers is transmitted directly to the treatment site and/or from the transducer to the treatment site through an energy conduit, for example an optical fiber. In certain embodiments the distal end of the energy conduit is configured as a diffuser. The light sources and/or ends of the energy conduits optionally have diffusers. The diffusers are geometric configurations designed to diffuse the energy emitted by the light source 57. In certain embodiments the diffuser (not shown) has a semi-circular or otherwise convex cross-section. The diffuser is aligned with the light source 57.

The illuminating device 57 has one or more light sources 57A. The light sources 57A are in, and/or on, and/or adjacent to the fluid outlets 58A. In certain embodiments one or more of the fluid and/or light controls are controlled by electronic (e.g., microprocessor, timer) and/or mechanical components located in the cleaning system, for example in the head.

In certain embodiments the head 55 is configured to be the fluid control and/or light control when an external force is applied to press the head 55 toward the cartridge 32. The head 55 is integral with or attached to, and in fluid communication with, the valve (not shown). In certain embodiments one or more tabs (not shown) are configured to fixedly or removably attach the head 55 to the cartridge 32. The tabs are integral with and or removably attached to the cartridge 32 and/or the head 55. The delivery conduit 37B is integral with or fixedly or removably attached to the head 55, for example through a connector (not shown). In certain embodiments the one or more fluid outlets 58A are configured to introduce turbulence (e.g., to introduce air into the photosensitizer solution) to the exiting fluid flow. For example, the fluid outlet 58A is partially blocked and/or is carbureted. In certain embodiments each fluid outlet 58A is configured to deliver a different fluid.

FIG. 10 illustrates that the delivery conduit 37B has a neck 61. The neck 61 is articulatable, angled, curved, or has other characteristics described herein.

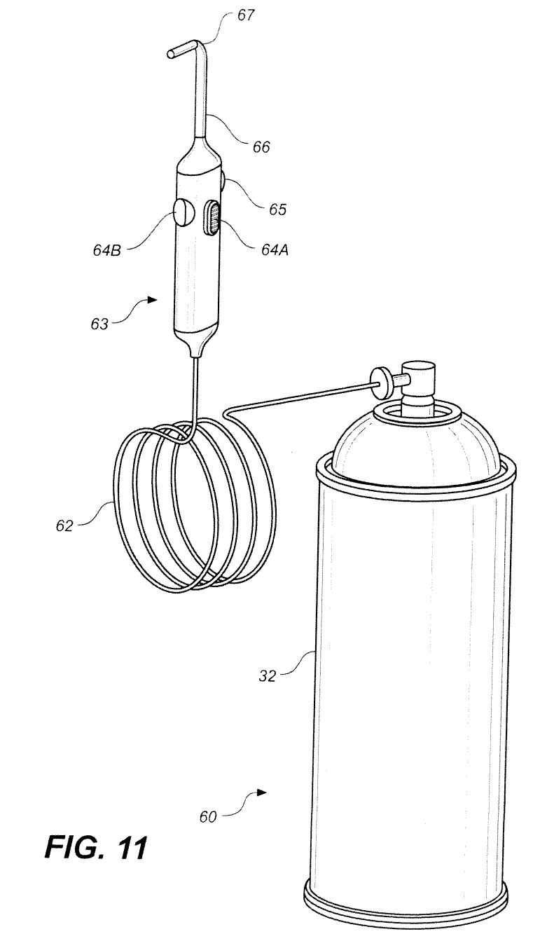

FIG. 11 illustrates a cleaning system 60 in which the distal end of the delivery conduit 62 is integral with and/or fixedly or removably attached to an applicator 63. The applicator 63 has one or more applicator-based fluid controls 64A and 64B. The fluid controls 64A and 64B are configured to activate the flow and/or control the flow rate, the flow turbulence (i.e., controlling laminar flow or Reynolds number of the flow), the ratio of different fluids, the aeration, and combinations thereof. The fluid controls 64A and 64B, and/or any light controls, are momentary (i.e., defaults to an inactive setting and needs to have an external, such as a manual, force applied to remain in an active position), bi-stable (e.g., stable in on and off settings), tri-stable, quad-stable, analog, separate controls, combined into a single control, or combinations thereof. In certain embodiments the applicator 63 has a power source (not shown), for example an electrical cell (i.e., battery) or a connector to an external electrical supply (e.g., an electrical cord or wire and plug).

The applicator 63 is integral with, or fixedly or removably attached to, one or more tips 66. The tip 66 is an integral part of, or fixedly or removably attached to, the delivery conduit 62. The tip 66 is a hollow conduit having a neck 67. In certain embodiments the cross-section of the tip 66 is configured substantially equivalent to the cross-section of the delivery conduit 62.

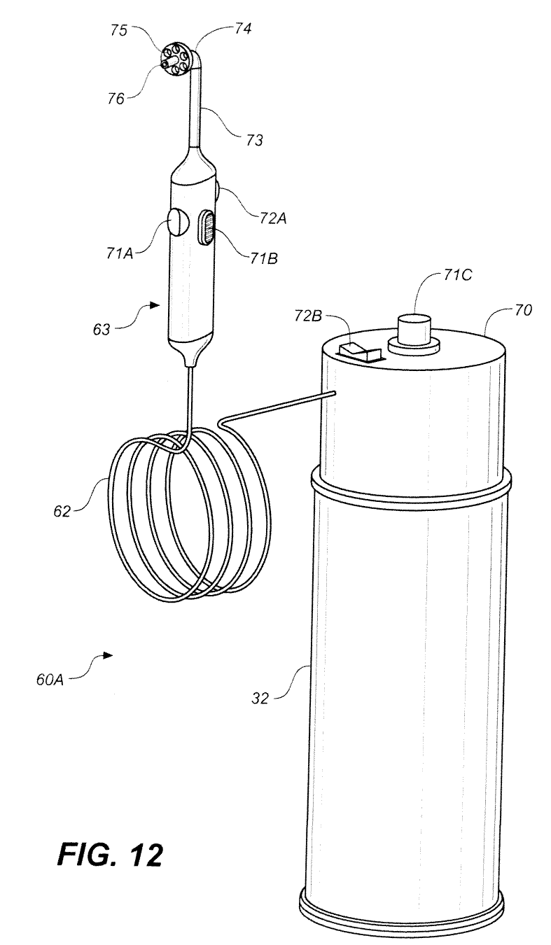

FIG. 12 illustrates that in certain embodiments the head 70 is configured as a cylindrical cap on the cartridge 32. The head 70 is optionally attached to the cartridge 32 by an interference fit. One or more tabs (not shown) are configured to fixedly or removably attach the head 70 to the cartridge 32. The tabs are integral with and or removably attached to the cartridge 32 and/or the head 70. The cleaning system 60A has the fluid controls 71A, 71B and 71C and/or light controls 72A and 72B on the applicator 63 and/or on the head 70. In certain embodiments the fluid controls 71A and/or 71B on the applicator 63 are in mechanical, electrical, data, or other controlling communication with the fluid control 71C on the head 70. In certain embodiments the light control 72A on the applicator 63 is in mechanical, electrical, data, or other controlling communication with the light control 72B on the head 70. The fluid control 71C is directly engaged with the valve (not shown). A mechanical or electronic timer (not shown) is optionally used, for example in conjunction with the fluid controls 71A, 71B or 71C and/or light controls 72A or 72B to control the fluid flow and illumination, for example to control and/or limit duration of flow and/or illumination.

In certain embodiments illumination is activated by fluid flow. For example, by a mechanical and/or electrical switch in the delivery conduit 62. For example, when the illumination is activated by fluid flow, the illumination duration is controlled through a mechanical or electronic timer.

The tip 73 optionally has one or more light sources 75, or portions of light sources 75. The light sources 75 are integral with, or fixedly or removably attached to the tip 73. In certain embodiments the light sources 75 are configured circumferentially around the fluid outlet 76.

The head 70 and/or applicator 63 optionally have a power source (not shown), for example, an electrical cell (i.e., battery) or a connector to an external electrical supply (e.g., an electrical cord or wire and plug). In certain embodiments the head is in electrical, and/or RF communication with the applicator 63 and/or the light sources 75. One or more RF generators (e.g., LEDs) are in the head 70 and/or the applicator 63 (e.g., in the tip 73, for example at the light source 75). The RF generators are in RF communication with the applicator 63 and/or light sources 75.

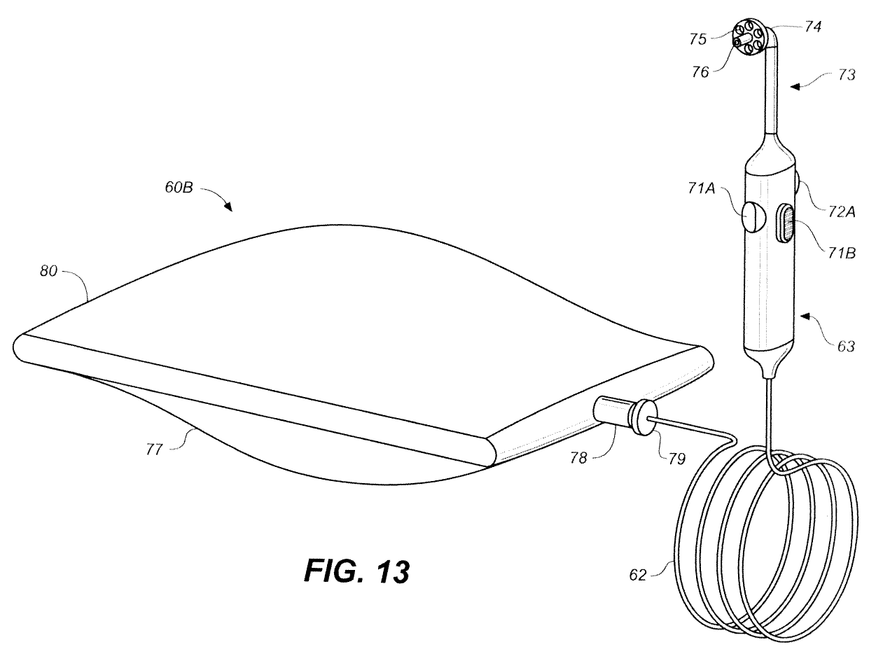

FIG. 13 illustrates that cleaning system 60B has the bladder 77 sans the cartridge e.g., 32. The bladder 77 has the head 78. The head 78 contains a valve that ensures containment of the bladder contents when the bladder 77 is not connected to the delivery conduit 62. The bladder 77 is fixedly or removably attached, for example through a connector 79, to the delivery conduit 62 via the head 78. The delivery conduit 62 is connected to the head 78 so the contents of the bladder 77 is in fluid communication with the delivery conduit 62, for example, allowing control of the sensitizer solution delivery to be accomplished through the fluid controls of the applicator. The head 78 reinforces the bladder 77. In certain embodiments the bladder has an internal gas pressure greater than the ambient pressure. The internal gas pressure pushes the sensitizer solution out of the bladder 77, for example through the head 78. A pressure regulating device (not shown), that is either manually or automatically controlled, such as a variable resistance valve (e.g., a spring loaded butterfly valve) varies the resistance of the fluid path to compensate for reducing gas pressure as the sensitizer solution leaves the bladder 77.

In certain embodiments the bladder 77 is mechanically squeezed to force the sensitizer solution 80 out of the bladder 77, for example through the use of an inflatable pressure cuff (not shown). Varying the mechanical force allows for flow adjustability and/or the maintenance of uniform flow through compensation for decreased pressure due to fluid leaving the container.

A pump (not shown) is used to pressurize the sensitizer solution 80. A pump (not shown), and/or container of pressurized gas, for example a gas high in oxygen concentration, is used to pressurize the bladder 77. A variable resistance and active or passive control system is used to control and/or maintain the fluid delivery pressure and/or sensitizer solution flow rate.

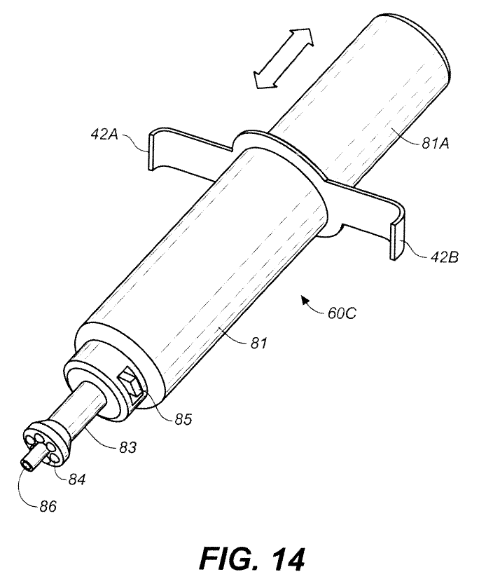

FIG. 14 illustrates that the head 81 is slidably attached, as shown by arrows, to the cartridge 81A similar to the head 41 and cartridge 41A of the fluid delivery system 40 in FIG. 7. As shown in FIG. 14, the delivery conduit 83 has one or more transducers 84, for example light sources 84, optionally configured circumferentially around the fluid outlet 86. In certain embodiments the cartridge 81A and/or head 81 are configured to have one or more transducers 84 as well as one or more power cells (not shown), for example batteries.

The head 81 optionally has a light control 85. The light control 85 is configured to activate, and/or deactivate, and/or control the energy emission profile of the light source 84. In certain embodiments the light control 85 is located on the bottom surface of the grip 42A and/or 42B so as to be activated when force is applied to cause the slidable motion of the cartridge 81A. In certain embodiments the light source 84 is activated and deactivated, for example, respectively by the inward and outward slidable motion of the cartridge 81A.

In certain embodiments the light source 84 is activated by the flow or pressure of the sensitizer solution (not shown) as it is released from the cartridge 81A. The light source 84 is deactivated by the cessation of flow or pressure from the cartridge 81A. In certain embodiments the slidable motion of the cartridge 81A activates a control mechanism, for example a switch (not shown), inside the head 81 that controls the activation and/or deactivation of the light source 84.

In certain embodiments the head 81 contains a variable resistance and/or active or passive control system (not shown) to control and/or maintain the fluid delivery pressure, and/or sensitizer solution flow rate, and/or activation and deactivation of the light source. The control system is used to decouple the deactivation of the light source from the slidable motion of the cartridge, allowing, for example, the light source to remain on for a predetermined or adjustable period of time after the cessation of sensitizer flow.

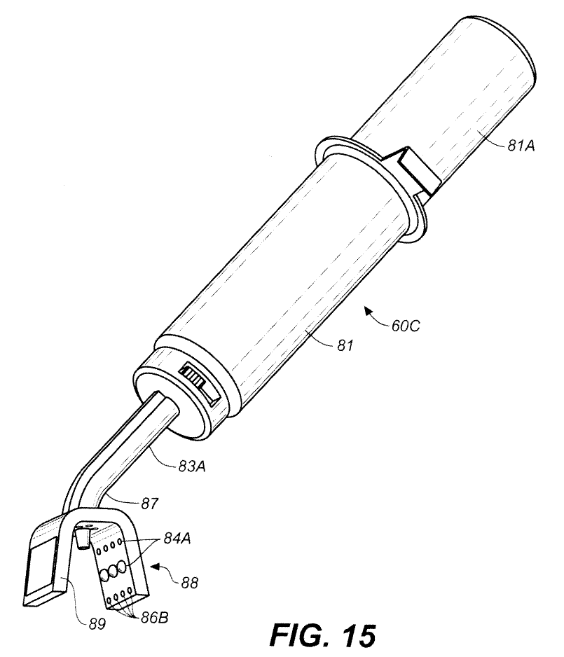

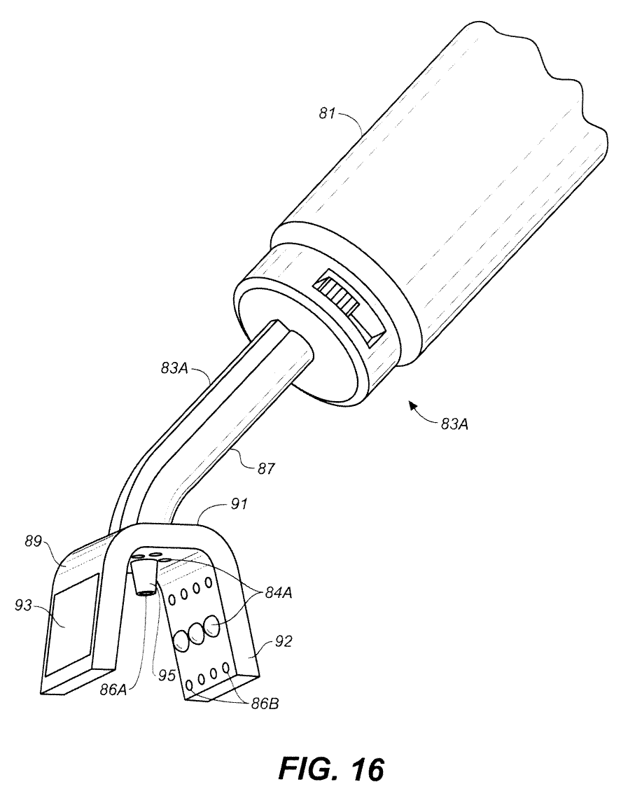

FIGS. 15 and 16 illustrate that in certain embodiments the applicator 88 has a shaped delivery body 89 near the end of the delivery conduit 83A. The body has a connecting plate 91 from tube 87 and sidewalls 92 to direct the application of sensitizer solution and/or energy from the transducers 93. The body 89 is in electrical and/or RF connection with the head 81 and or cartridge. The body 89 optionally has holes, channels, notches, grooves or a combination thereof to direct the flow of the sensitizer solution, for example, in response to a magnetic and/or electric field. The delivery conduit 83A optionally has a tip 95 for fluid delivery at 86A. The tip 95 extends beyond the body. Any body surface optionally has multiple fluid outlets. The surface of the body 89, in whole or in part, has features and/or materials that aid in the removal of biofilm (plaque) and/or in the distribution and/or activation of sensitizer solution. In certain embodiments the surface of the body 89 has soft polymer bristles similar to those found on a toothbrush, and/or closed or open loop material, and/or polymer foam (e.g., open cell, closed cell), and/or a non-soluble gel, or combinations thereof. The surface features and/or materials are optionally transparent and/or transmissive and/or conductive to the energy emissions of the transducers, for example, the surface has polymer bristles that can transmit light energy and mechanical energy.

FIGS. 17 and 18 illustrate that the in certain embodiments the body has a magnetic field generator. The magnetic field generator optionally contains zero, one, or more than one permanent magnets 94 alone or in combination with zero, one, or more than one transducers 90 to create a magnetic field, for example electromagnets (e.g., conductive material, for example wire, formed into a loop, for example, a circle or helix, with current flowing through the loop). The magnetic field generator optionally includes a control system. The control system optionally includes manual and/or automatic controls and/or sensors. The controls adjust characteristics of the magnetic field (e.g., orientation, intensity, flux density, rate of change, duration, pulse rate). In certain embodiments feedback from sensors is used to automatically adjust the characteristics of the magnetic field, and/or to alert the user of recommended action(s). The intensity and orientation of the magnetic field is controlled by controlling, for example the amount and direction of current flowing through the magnetic field generator, the number of turns of wire, the shape of the conducting loop, the properties of the materials in and around the conducting loop, or combinations thereof. The magnetic field is constant or variable. In certain embodiments the magnetic field is a combination of constant and variable magnetic fields. In certain embodiments the magnetic field is reversible. In certain embodiments the sensitizer solution and/or naturally occurring fluids found in the treatment site contain compounds that experience a force when exposed to a magnetic field (i.e., magnetic particles). The force applied by the magnetic field results in changes in the orientation of the magnetic particles. Magnetic particles that are mobile experience motion as a result of the force created by the electric field. In certain embodiments the transducers are positioned, and the direction, magnitude, and timing of the electric current is controlled, to force any magnetic particles in the area of the magnetic field to become oriented (e.g., line up, align) and/or move in a particular direction and/or pattern, for example to circulate and/or to oscillate toward or away from the treatment site. The flow of solutions in the treatment site as a result of a magnetic field are optionally directed, and/or encourage, and/or inhibited and/or otherwise controlled by features of the cleaning system (e.g., holes, channels, notches, grooves, protrusions) that allow or inhibit solution motion. The motion and/or orientation of magnetic particles, due to the presence of a magnetic field, directly result in the motion and/or orientation of non-charged particle in the solution and/or other fluids in the treatment site due to the properties of the fluids (e.g., viscosity). In certain embodiments the orientation, magnitude, and timing of the magnetic field is controlled, for example, to force magnetic sensitizer particles to orient themselves so as to increase their likelihood of absorbing incoming energy, and therefore increase their quantum yield of RCS. In certain embodiments a magnetic field created by a device, for example a permanent magnet and/or electromagnet, separate from the solution delivery system is used in combination with the solution delivery system and/or the sensitizer solution alone. The separate magnetic field generating device is, for example, in the form of a handpiece used to apply a magnetic field externally, a catheter designed to access and apply a magnetic field to an internal body surface, and/or or a stationary device, for example a magnetic resonance imaging system. The applied magnetic field increases the penetration of the sensitizer solution, or some of its compounds (e.g., those that respond most strongly to the magnetic field), into small spaces (e.g., between teeth, between the teeth and gums, into the alveoli of the lungs), into biofilms, into and/or through pores (e.g., pores in bacterial or other organism cell walls and/or membranes, pores in the skin or mucosal surfaces), into porous surfaces (e.g., tooth enamel, tooth dentin, finger and/or toe nails), under toe nails, into the base of hair follicles, into atherosclerotic materials (e.g., arterial plaque), between the villi of the linings of the digestive tract, and through cell layers and/or membranes (e.g., endothelium, mesothelium, basil lamina, skin). In certain embodiments motion of solution compounds (e.g., sensitizer, compounds that are consumed in chemical reactions, oxygen, catalysts) as a result of the magnetic field results in higher concentrations of these molecules being located near and/or within the target sites and/or organisms, thereby increasing the effectiveness of the system at achieving the desired outcome (e.g., destruction of target organisms, tooth whitening, and/or bleaching). In certain embodiments the coils are designed to have a resistance so that current flow produces heat.

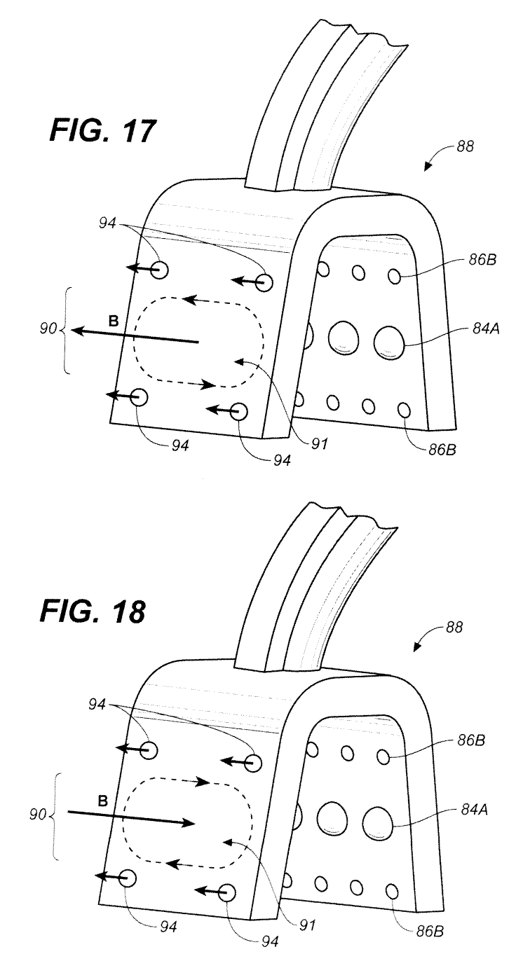

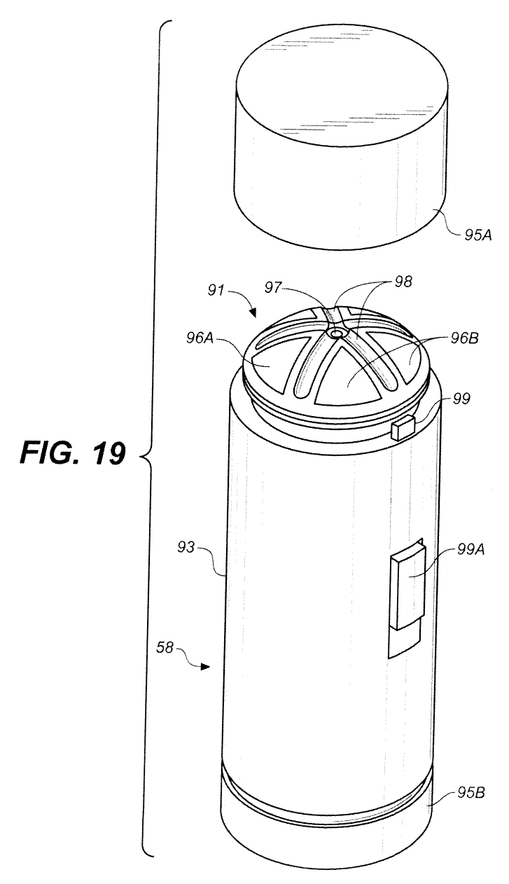

FIG. 19 illustrates that the cleaning system has a handle 93 that fully or partially (not shown) encloses the fluid cartridge (not shown) and a convex head 91 that is designed to aid in the application, and/or distribution, and/or activation of the sensitizer solution. The handle 93 is a hollow container. The handle 93 has a pressurized cartridge. In certain embodiments the cartridge has a frangible seal (no valve). In certain embodiments the head 91 and/or handle 93 have a valve in fluid communication with the frangible seal (not shown). Inserting the cartridge into the handle 93 and/or putting the end cap 95A or 95B on the handle 93 places the cartridge contents in fluid communication with the valve. Pushing down on the head 91, or pushing a switch 99A (which would allow the head to be fixed), releases the sensitizer solution. In certain embodiments valves are in both the handle 93 and the head 91. In certain embodiments both switches (only one switch 99A shown) are activated concurrently (e.g., pushing a first switch 99A and pressing down on a second switch (not shown)). In certain embodiments release of the sensitizer solution requires that both switches be activated concurrently. In certain embodiments the cartridge has a valve with a frangible seal. The valve is optionally actuated by a switch 99A or by applying force to the head 91.

The cartridge is optionally slidably loaded into the top and/or bottom of the cleaning system 60D. Either end cap 95A or 95B is fixedly or removably attached to the handle 93. The head 91 and/or the handle 93 have batteries (not shown). The batteries are permanent or replaceable.

In certain embodiments the actuator 99A has an interlock 99. The cover 95 activates the interlock 99, for example, when the cover 95 is attached to the head 91 and/or handle 93. The interlock 99 prevents the switch 99A from being activated.

The switch 99A optionally controls the transducers directly or in certain embodiments via a timer or other electronic controller (not shown). In certain embodiments the head 91 has sensors that provide feedback to the controller.

In certain embodiments the cleaning system 60D has controls (not shown) to control sensitizer solution release and/or the time of illumination and/or which transducers are active and/or the characteristics of the energy emission profile of the active transducers 96A and 96B.

The cartridge can slidably load into the handle. In certain embodiments the fully-loaded cartridge is configured to protrude from the handle 93. In certain embodiments the cartridge (not shown) screws into the handle 93. The cartridge optionally attaches to a connector (not shown) on the head 91. In certain embodiments the cartridge has a connector (not shown) that attaches to the head 91.

In certain embodiments the handle 93 is the pressurized container. The handle 93 has the valve. The head 91 optionally has a spray nozzle. The head 91 optionally has two valves (not shown). Each valve is actuated by a separate switch. In certain embodiments either end cap and/or the handle have a filling and/or refilling port. In certain embodiments the cleaning system has auditory and/or visual outputs, for example, to let the user know that the treatment is done and/or that the transducer 96A and 96B is active. The head 91 has one or more fluid outlets 97. The fluid outlets are connected via internal channels (not shown). The head 91 optionally has surface features (e.g., depressions (e.g., channels, grooves, 98, dimples), protrusions (e.g., bumps, ridges), perforations) on all or a portion of the head's surface designed to improve the mobility, and/or application, and/or distribution, and/or mixing of the sensitizer solution compounds with each other or with compounds that are found at the treatment site (e.g., saliva, sweat, blood plasma, blood serum, interstitial fluid, mucous, urine, lymph, vaginal fluids, irrigation fluids (e.g., water, ringers lactate, saline, etc.).

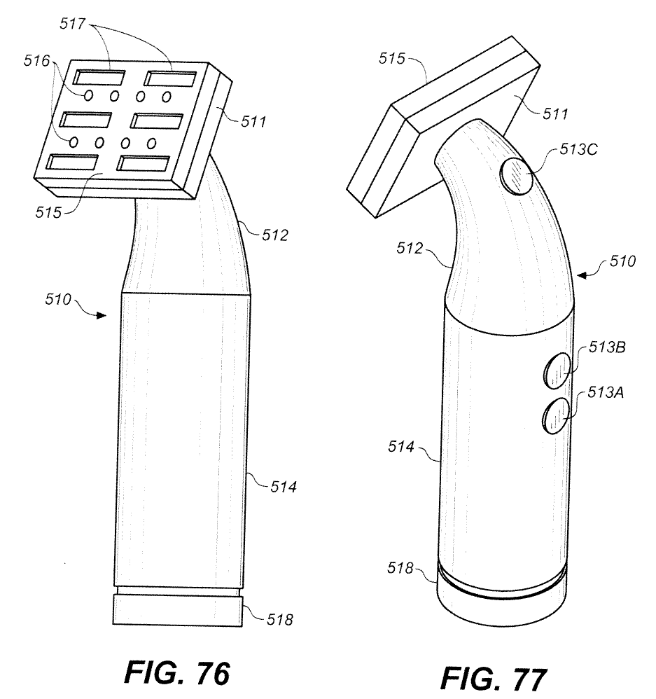

In certain embodiments the head 91 has one or more transducers. In certain embodiments the transducers 96A and 96B are an electrode and/or a magnetic field generator and/or heating element. All the electrodes or magnetic poles are optionally of the same type (e.g., +, −, North, South) or alternate over the surface of the head. In certain embodiments the pad (shown as a convex surface on the head) material is conductive (See FIG. 76 for alternate flat embodiment). In certain embodiments the pad is an electrode. The pad optionally has charged regions electrically isolated from each other. In certain embodiments the head 91 has one or more areas covered by one or more porous materials. The porous material is optionally a porous structure of natural and/or artificial material (e.g., open cell foam (e.g., polyvinyl alcohol (PVA), polyurethane, polyvinyl chloride (PVC), polyolefin, polystyrene), polymer matting and/or fabric, polytetrafluoroethylene (PTFE), expanded polytetrafluoroethylene (EPTFE)), a fluid impermeable material (e.g., metal foil, polymers, closed cell foam (e.g., polyvinyl alcohol (PVA), polyurethane, polyvinyl chloride (PVC), polyolefin, polystyrene), polytetrafluoroethylene (PTFE), expanded polytetrafluoroethylene (EPTFE)) or combinations thereof. The porous material has pores and/or fibers. The pores are air-filled. Between about 5% and 95% of the porous material volume is air. In certain embodiments the applicator has one or more permeable regions made impermeable, for example through the application of an impermeable coating such as Parylene. The porous material is optionally soft or abrasive and optionally contains abrasives (e.g., aluminum oxide, silicon dioxide). In certain embodiments the porous material is permanently or removably attached to the head and covers all or only a portion of the head. In certain embodiments the grooves have a fluid channel or one or more fluid outlets (e.g., at one end of the channel) and the porous material, such as a foam insert, is glued into the groove. In certain embodiments the sensitizer solution is released into the foam. The foam saturates with the sensitizer solution. The foam releases the sensitizer solution. In certain embodiments the head has fluid channels and/or openings that align with channels and/or openings in the foam. The foam optionally covers all or only a portion of the head. The pad is removably or fixedly attached to the head. In certain embodiments the pad is sterilized before being attached to the head. In certain embodiments the head and pad are sterilized together. The head is removably or fixedly attached to the handle. In certain embodiments the transducers 96A and 96B are located in the handle 93, for example, under the head 91. The head 91 is transparent and/or translucent and/or transmissive (e.g., electrically conductive). In certain embodiments the head 91 is connected to an interlock 99. The transducers are activated and/or the solution is delivered when the interlock 99 is activated. In certain embodiments the interlock (not shown) is on the handle 93 under the head 91, or in the head 91 facing either out toward the user or in toward the handle. The head is optionally round, flat, oval, oblong, or combinations thereof.

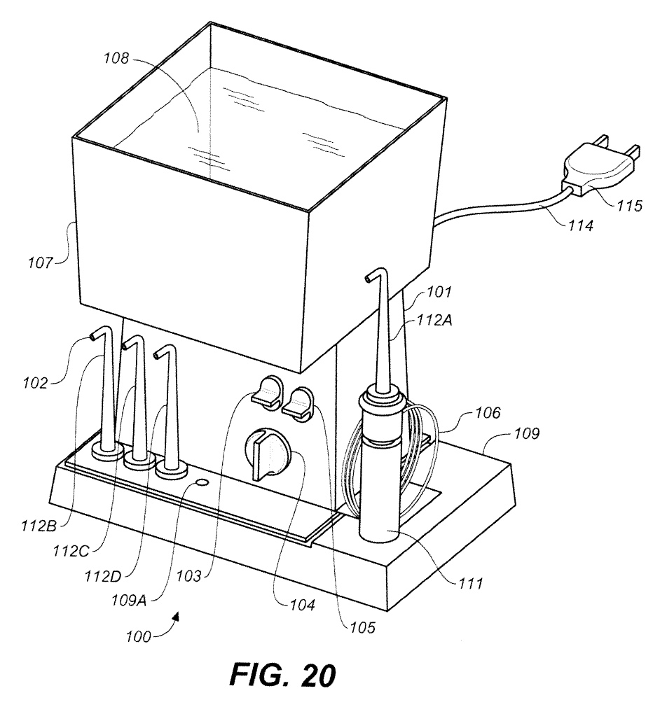

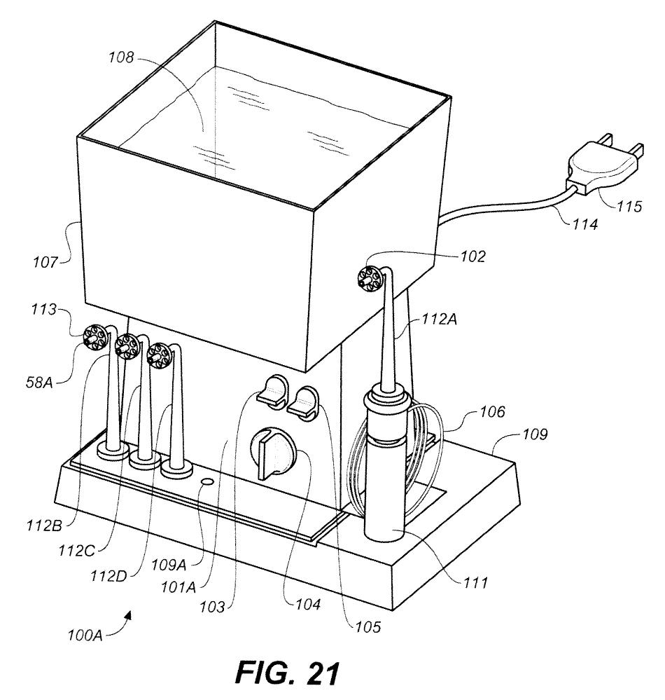

FIGS. 20 and 21 illustrate that in certain embodiments the fluid delivery system 100 and/or the cleaning system 100A have a solution delivery system (SDS) 101. The SDS 101 has one or more pumps (not shown), all electrical circuits and processors needed (not shown) for operation, and/or a system of flow channels and/or paths and/or mixing chambers (not shown), and/or connectors (not shown), and/or the transducers, such as an illuminating device, for example the light source 102, and/or one or more light controls 103, and/or one or more fluid controls 104, and/or one or more other controls 105 (e.g., system power control). The SDS 101 is in fluid communication with, and/or electrical communication with, and/or RF communication with, and/or ultrasound communication with, and/or integral with, or removably or fixedly attached to the delivery conduit 106 and/or the reservoir 107. In certain embodiments the reservoir 107 contains one or more transducers (not shown), for example, in the floor of the reservoir. In certain embodiments the SDS 101 contains one or more transducers (not shown), for example, in the top surface of the SDS located directly below the reservoir 107. The transducers are optionally electromagnetic and or ultrasonic energy transducers. In certain embodiments the reservoir 107, or a portion of the reservoir 107, for example, the floor and/or walls of the reservoir, is translucent or transparent to energy from the transducer. In certain embodiments the reservoir 107, or a portion of the reservoir 107, for example, the floor and/or walls and/or septa of the reservoir, transmits energy from the transducer into the interior of the reservoir 107, for example, into the sensitizer solution 108. The pump is configured to pump air, and/or fluid from the reservoir 107, and/or a fluid intake conduit, and/or a fluid container. The mixing chamber is used to mix any fluids (e.g., sensitizer solution, air, and oxygen).

A mixer is selected from a venturi, pump, mixing chamber, flow (e.g., conduit, tube, pipe) junctures, mixing geometries in conduits (e.g., tubes, pipe), and combinations thereof.

The fluid delivery system 101 or the cleaning system 100A has a base 109. The fluid delivery system 100 and/or the cleaning system 100A have tip ports 109A, for example, in the base 109. The tip port 109A is configured to releasably attach to one or more tips 112ff, for example as shown as second, third and fourth tips 112B, 112C, and 112D. The SDS 101 is configured to permanently or releasably attach to the delivery conduit 106. The delivery conduit 106 is configured to permanently or releasably attach to the applicator 111. The applicator 111 is configured to permanently or releasably attach to the tips 112ff. The tips 112ff are optionally configured to permanently or releasably attach to the light source 102, for example, to allow light sources of various frequencies, configurations (e.g., a lesser or greater number of transducers), and/or powers to be attached to the tip 112ff.

In certain embodiments the SDS 101 is attached to a power connector 114, such as an electric cord or wire 114 and plug 115. The power connector 114 is configured to deliver power to the pump, and/or illuminating device, and/or controls. In certain embodiments the SDS 101 has an on-board or external power source, for example, one or more electrical cells (i.e., batteries) contained within the cleaning system 101A. In certain embodiments the power connector receives power from mechanically captured power from the pressurized (e.g., pressure caused by manual pressurization or gravitation) release of photosensitizer solution 108 and/or water and/or another fluid and/or gas.

The reservoir 107 has the photosensitizer solution 108. The reservoir 107 is uncovered. The reservoir 107 is optionally configured to fit on a base 109, for example covering elements of the base (e.g., tips, controls) and preventing dust from collecting in the reservoir when the fluid delivery system 100 or cleaning system 100A is stored, for example, the reservoir is turned upside down and placed over the base 109, tips and controls.

The photosensitizer solution 108 is configured to be stored in the reservoir 107. The SDS 101 is configured to pump or otherwise direct flow of the photosensitizer solution 108 through the SDS 101, the delivery conduit 106 and/or the applicator 111. In certain embodiments the illuminating device is in or on the SDS 101 and light energy is delivered along and out the delivery conduit 106 and/or applicator 111, and/or the illuminating device 113 is optionally in or on the delivery conduit 106 and/or applicator 111.

The cleaning system 100A has one or more light sources 102 (113), for example at and/or proximal to the ends of the tips 112A, 112B, 112C and 112D. The light sources 102 are configured to be activated by the light control 103, and the behavior of the light source 102 (e.g., strobing, light frequency, intensity/brightness) is controlled by the light control 103.

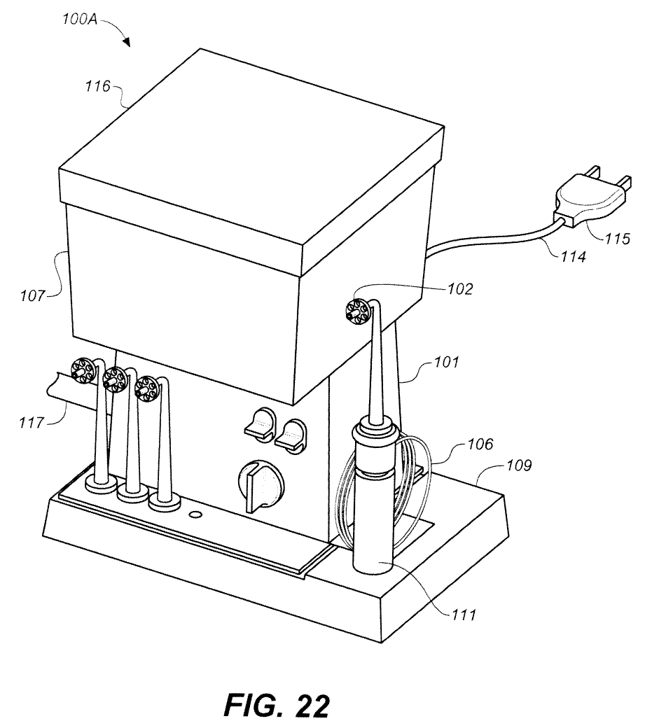

FIG. 22 illustrates that the reservoir 107 has a cover 116. The cover 116 is integral with, or fixedly or removably attached to the reservoir 107. The cover 116 forms a photosensitizer solution-tight and/or air-tight seal with the reservoir 107. In certain embodiments the reservoir 107 contains enough sensitizer solution (not shown) to complete multiple treatments. In certain embodiments the reservoir 107 contains sensitizer solution (not shown) that is in a concentrated form.

In certain embodiments one or more fluid intake conduits 117 are in fluid communication with the SDS 101 and/or the reservoir 107. The fluid intake conduits 117 are attached to a pressurized fluid and/or gas supply, for example, a pressurized container of photosensitizer solution, and/or a water faucet, for example, that is attached to a well-fed or municipal water supply. The fluid intake conduits 117 supply water and/or photosensitizer solution. The fluid intake conduits 117 provide a fluid that can mix in the SDS 101, and/or in the reservoir 107, and/or in the delivery conduit 106, and/or in or distal to the applicator 111 with the contents of the reservoir 107.

In certain embodiments the fluid intake conduits 117 provide a fluid that provides energy to create or supplement the fluid flow in the delivery conduit. For example, the fluid from the fluid intake conduit 117 enters a channel in the SDS 101 that is configured to drive a water wheel, and/or create pressure, and/or create a vacuum, for example when flowing through a venturi.

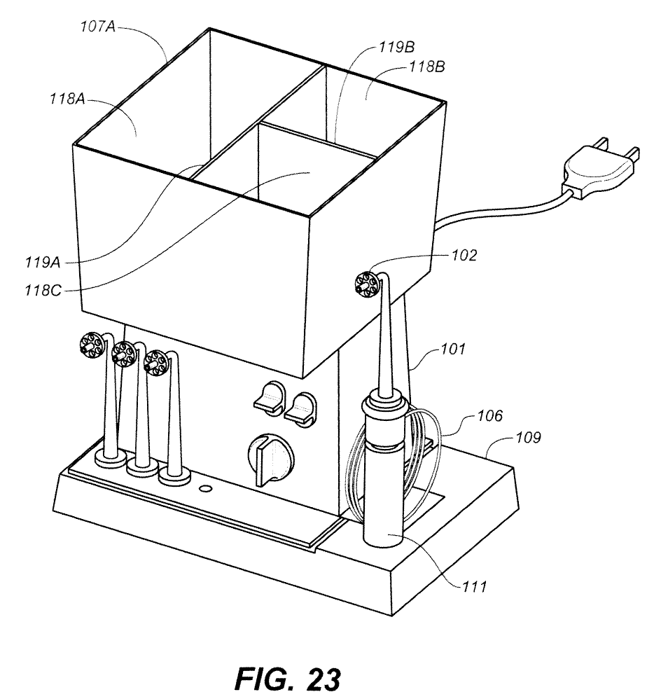

FIG. 23 illustrates that in certain embodiments the reservoir 107A has multiple chambers 118A, 118B and 118C separated by one or more reservoir septa 119A and 119B. In certain embodiments the reservoir 107A has a first chamber 118A and a second chamber 118B. In certain embodiments the reservoir 107A has a third chamber 118C. A first reservoir septum 119A fluidly isolates the first chamber 118A from the second chamber 118B. The first reservoir septum 119A and/or a second reservoir septum 119B fluidly isolates the first chamber 118A from the third reservoir chamber 118C. The first reservoir septum 119A and/or the second reservoir septum 119B fluidly isolates the second chamber 118B from the third chamber 118C. In certain embodiments the reservoir septa 119A and 119B are configured to rotate or otherwise establish a path of fluid communication between the chambers they separate, for example by way of a valve, to allow controlled mixing between the contents of all or any specific combination of the chambers. In certain embodiments the first and/or second and/or third reservoir septa 119A, and/or 119B and/or more are made from more than one openable (e.g., rotatable) sections and/or contain one or more valves, for example a first septum section (not shown) is opened to place the first chamber 118A in fluid communication with the second chamber 118B, and a second septum section (not shown) is opened to place the first chamber 118A in fluid communication with the third chamber 118C.

The septa 119A and 119B are optionally used in combination with the cover 116 (not shown). The septa 119A and 119B can form a sensitizer solution-tight and/or air-tight seal with the reservoir 107A and/or the cover 116.

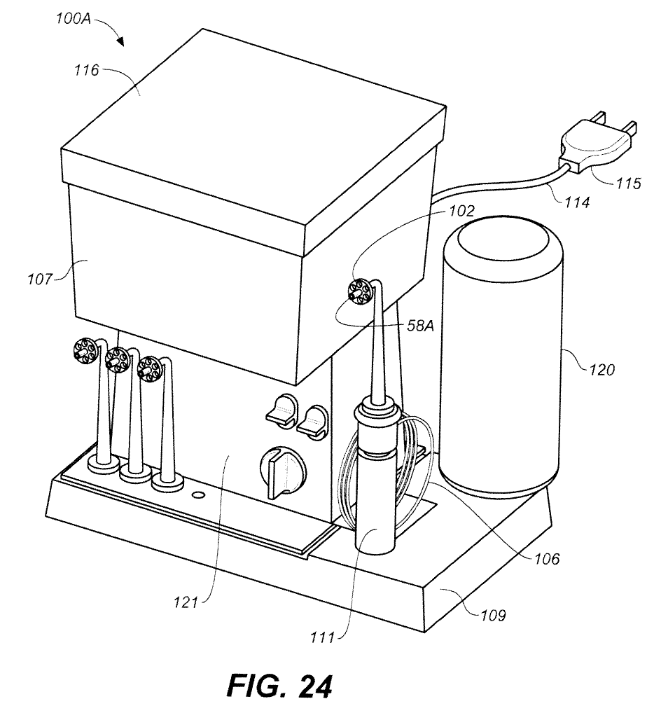

FIG. 24 illustrates that the fluid container is optionally a cartridge 120 in fluid communication with the SDS 121. The cartridge 120 is integral with, or releasably or fixedly attached to the SDS 121, for example, with or to a cartridge connector on the SDS 121. In certain embodiments the cartridge 120 contains pressurized or unpressurized sensitizer fluid. In certain embodiments the cartridge 120 contains air, oxygen, photosensitizer solution, and/or any other liquid and/or gas material described herein or combinations thereof.

In certain embodiments the cartridge 120 has a modular seal, for example a seal that is opened and closed by the cartridge connector on the SDS 121. In certain embodiments the cartridge 120 has a seal that is frangible or breakable, for example a thin metal layer or foil. In certain embodiments the cartridge 120 contains an ampoule, for example a breakable ampoule (e.g., made of glass and/or hard plastic).

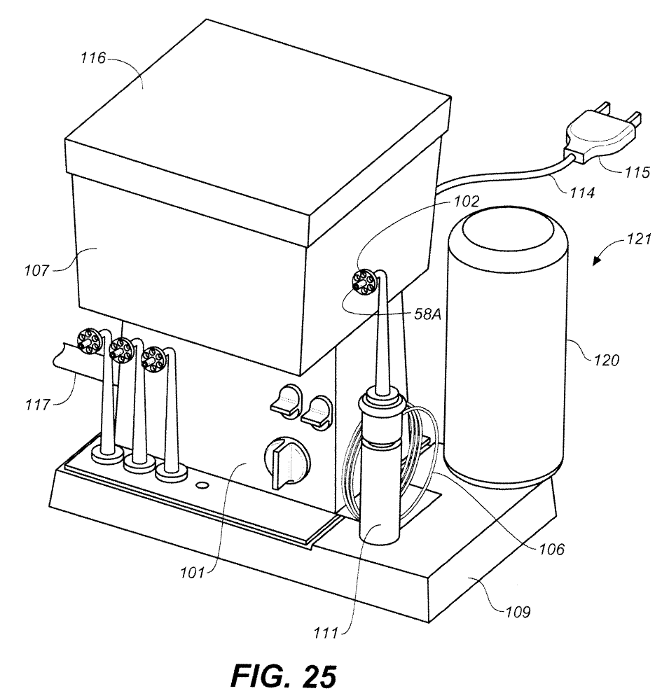

FIG. 25 illustrates that in certain embodiments the SDS 101 and/or the reservoir 107 are in concurrent fluid communication with the cartridge 120 and the fluid intake conduit 117. The cleaning system 121 optionally mixes the contents of the cartridge 120, and/or the contents of the fluid intake conduit 117, and/or the contents of the reservoir 107 in any combination in the SDS 101, and/or in the delivery conduit 106, and/or in the applicator 111, and/or in the cartridge 120.

In certain embodiments the cartridge 120 has a dissolvable material. The cartridge receives a fluid, for example that dissolves the dissolvable material.

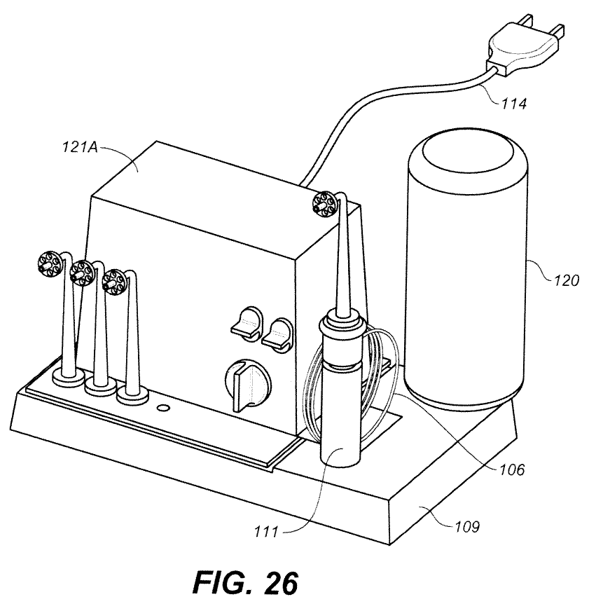

FIGS. 20, 21 and 23 illustrate that in certain embodiments the SDS 101 is in fluid communication solely with the reservoir 107 and the delivery conduit 106. In certain embodiments the SDS 101 is in fluid communication solely with the fluid intake conduit 117 and the delivery conduit 106. FIG. 25 illustrates that the SDS 101 is in fluid communication solely with the cartridge 120, the reservoir 107, the fluid intake conduit 117, and the delivery conduit 106, and that the cover 116 is integral with, and or fixedly or releasably attached to the reservoir 107. FIG. 26 illustrates that the SDS 121A is in fluid communication solely with the cartridge 120 and the delivery conduit 106. FIG. 27 illustrates that the SDS 121B is in fluid communication solely with the cartridge, the fluid intake conduit 117, and the delivery conduit 106.

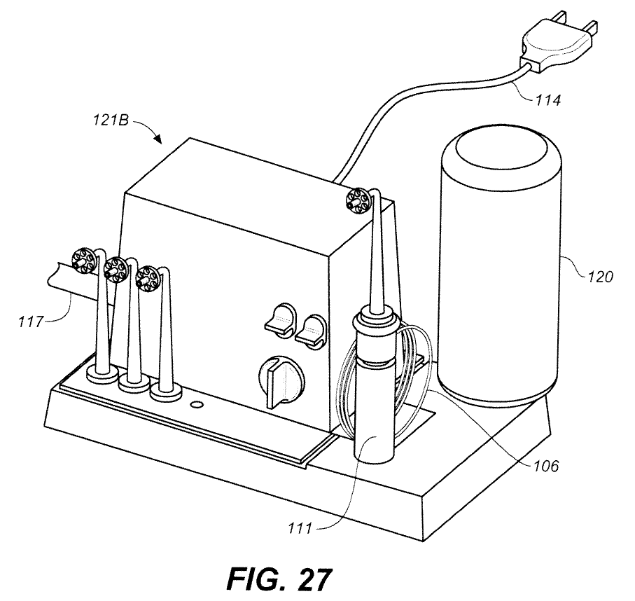

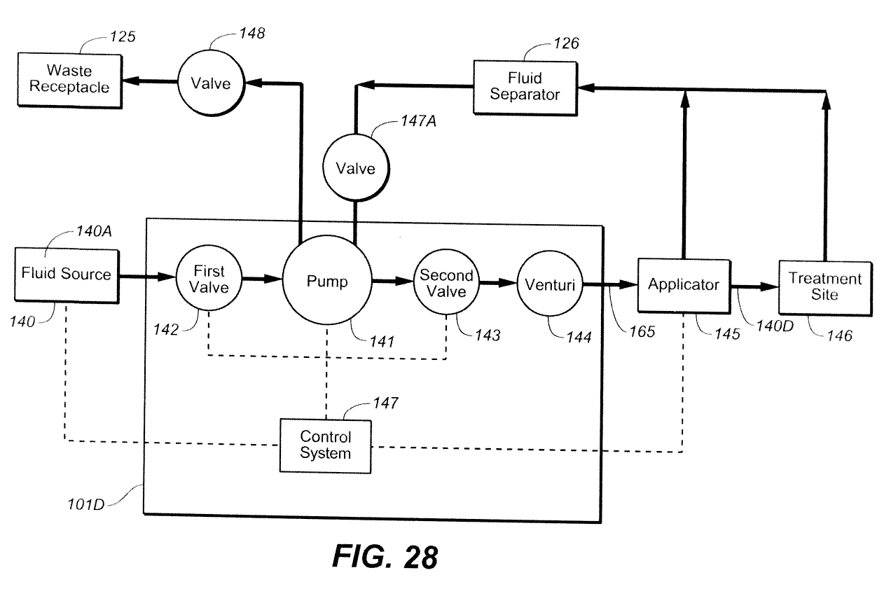

FIGS. 28 through 33 have similar features and are discussed briefly below. In certain embodiments vacuum is applied to the treatment site from a perforated tube, and/or from the applicator. The pump is optionally configured so that both sides (i.e., the top and the bottom, assuming the piston moves up and down) move fluid. As shown in FIG. 28, the top creates pressure to pump the solution and creates a vacuum to suck solution. The pump has a one-way valve to let fluid flow in only the desired direction.

The waste receptacle 125 is optionally a separate reservoir (e.g., a separate container or one of the reservoir chambers of the SDS, a sink or toilet).

In certain embodiments a fluid separator 126 is used. The fluid separator 126 optionally contains chemistry that renders the treatment solution inert and/or non-toxic. In certain embodiments the fluid separator 126 and/or the SDS have an energy source that renders the sensitizer inert or non-toxic. For example, the fluid separator 126 has a light source whose energy emission profile breaks chemical bonds in the sensitizer thereby destroying the sensitizer's ability to act as a photosensitizer. In certain embodiments fluid flow through the venturi draws air into the fluid stream increasing the dissolved gas (i.e., oxygen) concentration in the fluid stream.

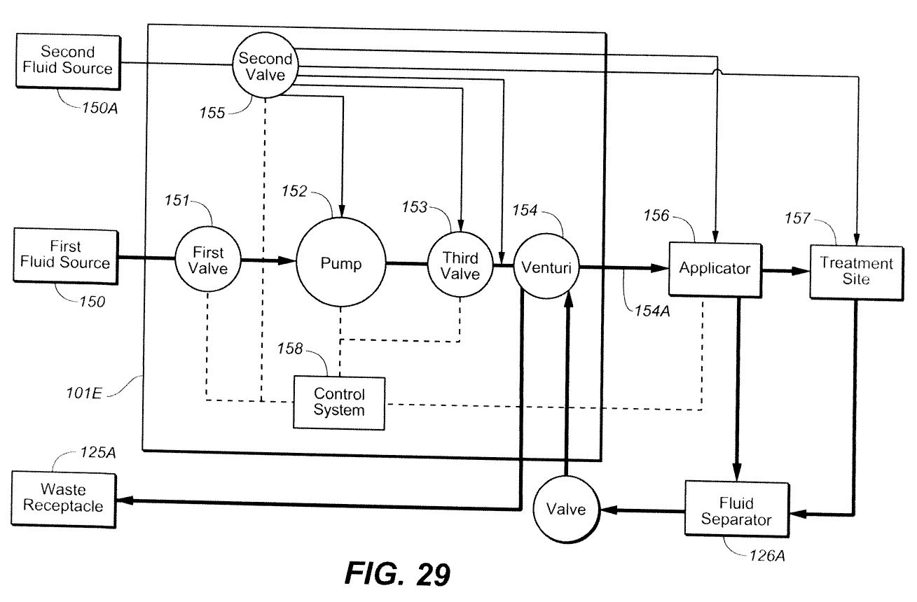

As shown in FIG. 29, in certain embodiments vacuum is created when fluid flows through the venturi. For example, fluid flow from a faucet enters the fluid intake conduit, goes through the venturi, and then into a waste receptacle 125A. Water is optionally delivered to the treatment site. The fluid separator 126A prevents waste fluid from entering the stream of fluid going to the treatment site. In certain embodiments a second venturi is added and the flow from the fluid sources is split between the two venturis.

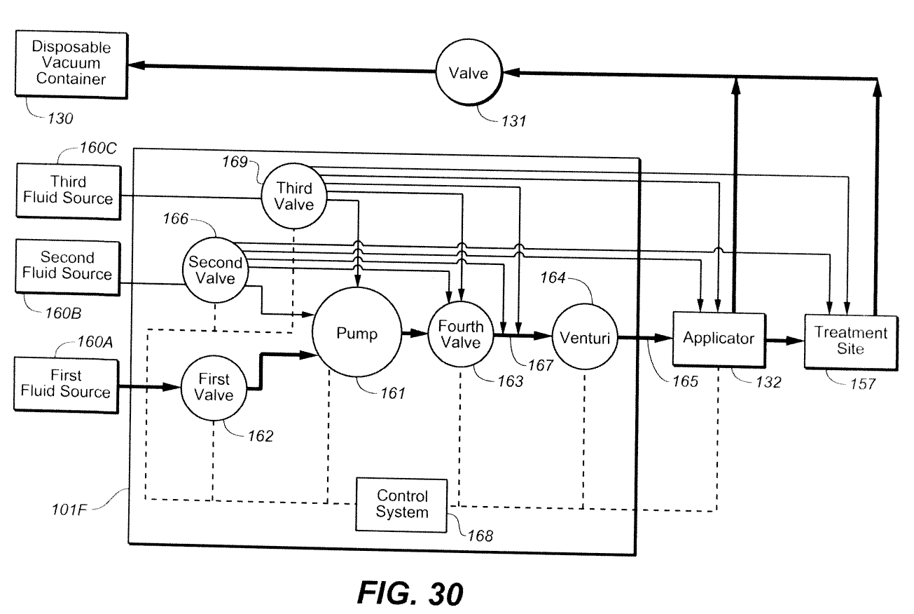

As shown in FIG. 30, the cleaning system has an external vacuum source 130. In certain embodiments a disposable vacuum container 130, such as an evacuated container, provides the vacuum. For example, a one-liter glass bottle that has an internal pressure of close to zero, or about −14.7 psig, is connected to the SDS 101F. The bottle is in fluid communication with a vacuum valve 131 and the applicator 132 or a separate suction tube at the treatment site 157. Opening the vacuum valve 131 exposes the treatment site 157 to the vacuum. Fluid is sucked into the disposable vacuum container 130.

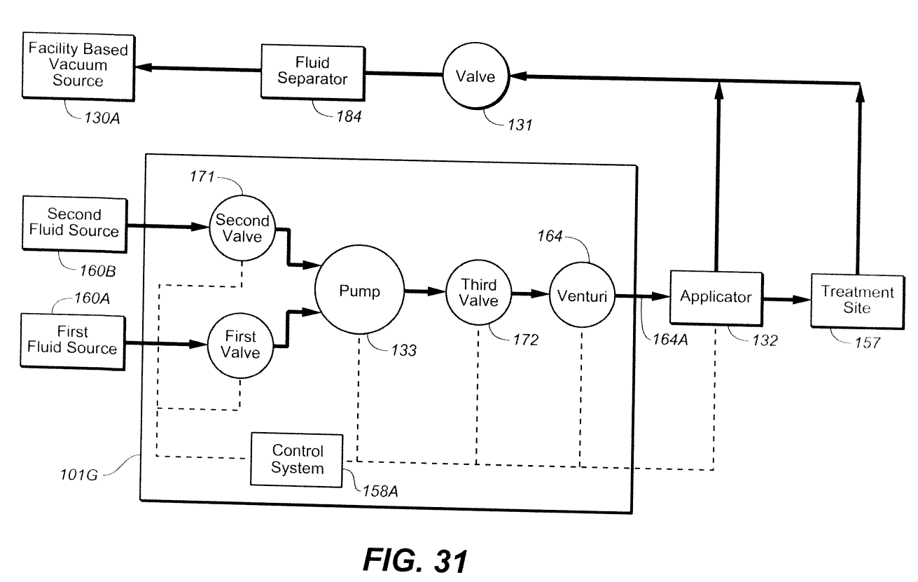

As shown in FIG. 31, in certain embodiments the cleaning system has a fixed vacuum pump 130A that provides vacuum. The vacuum pump 130A is separate from the SDS 101G. The vacuum pump 130A is separate from the cleaning system. In certain embodiments the SDS 101G or applicator 132 has a separate control and/or valve to control the vacuum level.

FIG. 28 schematically illustrates that the fluid source 140, for example the reservoir or the cartridge, is in fluid communication (shown by solid lines) with the SDS 101D. The SDS 101D is configured for the fluid source 140 to be in fluid communication with a pump 141, for example through a first valve 142. The first valve 142 has fully and/or partially open configurations, and a closed configuration. The pump 141 pressurizes the photosensitizer solution 140A from the fluid source 140.

The pump 141 is optionally in fluid communication with a mixing device, for example through a second valve 143. The mixing device is, for example, a venturi 144 or a fluid path geometry that results in turbulent fluid flow. The second valve 143 has fully and/or partially open configurations and a closed configuration. In certain embodiments the venturi 144 is configured to receive a supplemental fluid, such as those described herein including air or oxygen, for example from the surrounding environment. The venturi 144 is configured to mix the photosensitizer solution 140A and the supplemental fluid into a mixed photosensitizer solution. The venturi 144 is in fluid communication with the applicator 145 through, for example the delivery conduit. The applicator 145 delivers the photosensitizer solution 140D to the treatment site 146.

A control system 147 is in electronic and/or mechanical (e.g., pneumatic, hydraulic, linkaged) and/or data communication (shown by phantom lines) with the incoming photosensitizer solution 140A, and/or the first valve 142, and/or the pump 141, and/or the second valve 143, and/or the mixing device 144, and/or the applicator 145. In certain embodiments the control system 147 is in electronic and/or mechanical (e.g., pneumatic, hydraulic, linkaged) and/or data communication with sensors (e.g., for pressure, temperature, flow rate, chemical content such as sensitizer and/or pH and/or oxygen concentration) at, on, in, and/or adjacent to the incoming photosensitizer solution 140A, and/or the first valve 142, and/or the pump 141, and/or the second valve 143, and/or the mixing device 144, and/or the applicator 145, and/or the treatment site 146, and/or other components of the SDS 101D or fluid delivery system and/or cleaning system. The first valve 142 and/or the second valve 143 are optionally variable resistance valves and the level of resistance is controlled manually and/or by the control system 147.

In certain embodiments the control system 147 is configured to release or otherwise control flow of the incoming photosensitizer solution 140A into the SDS 101, for example by placing the first valve 142 in an open configuration and activating the pump 141. The control system 147 is configured to controllably place the first and/or second valves 142 and/or 143 in open and/or closed configurations. The control system 147 is configured to control the amount of pressure created by the pump 141, for example by activating and/or deactivating the pump 141.

The control system is optionally in communication with a pressure sensor. The pressure sensor detects over-pressurization in the SDS. The SDS optionally has a pressure relief valve (not shown). The pressure relief valve is activated by the control system, for example when over-pressurization is detected.

In certain embodiments the control system 147 is configured to control the applicator 145, for example by controlling the amount of light from the light source, such as by activating and/or deactivation the light source and/or controlling the voltage and/or current applied to the light source. In certain embodiments the applicator has an applicator valve (not shown). The applicator valve has fully and/or partially open and closed positions. The applicator valve optionally is a variable resistance valve. The control system 147 is configured to control the amount of fluid flow from the applicator 145, such as by activating and/or deactivating the fluid source 140 and/or pump 141 and/or controlling the position of the valves. The control system 147 is optionally configured to control the mixing device, for example by increasing the fluid flow to the mixing device and/or by reducing the mixing rate of the mixing device. The control system 147 controls the ratio of any inbound fluids to the SDS 101D that are present in any outbound fluids from the SDS 101D, for example through control of the mixing device and/or the first valve 142, second valve 143 or applicator valve (not shown).

In certain embodiments the control system 147 has a controller, such as a microprocessor. The control system 147 is in data communication with the fluid control and/or the light control, and/or the other controls. In certain embodiments the data communication between the control system and any controls is bi-directional. In certain embodiments the control system 147 has a pre-programmed controller. The control system optionally has knobs, switches, dials, levers, toggles, tabs, buttons, slides, accelerometers, fluid or contact pressure sensors, other rotating switches, other translating switches, or combinations thereof that are accessed by the user. Control variables of the control system are optionally preset and/or adjustable by the user.

FIG. 29 illustrates that a first fluid source 150 and/or a second fluid source 150A are in fluid communication with the SDS 101E. The first and/or second fluid sources 150 and/or 150A are optionally pressurized, such as pressurized cartridges. The first fluid source 150 is in fluid communication with the pump 152, the first valve 151, the third valve 153, the venturi 154, the delivery conduit 154A, and the applicator 156, similar to the fluid from the fluid source 140 as shown in FIG. 28 (with the second valve 143 in the SDS 101D in FIG. 28 being the third valve 153 in the SDS 101E in FIG. 29). The second fluid source 150A is in fluid communication with the second valve 155. The second valve 155 is in a fixed or a variable configuration to place the second fluid source 150A in fluid communication with the pump 152 and/or the third valve 153 and/or the flow channel of the SDS 101E between the third valve 153 and the venturi 154, and/or the delivery conduit 154A, and/or the applicator 156, and/or directly with the treatment site 157. Any of the aforementioned elements is considered to be the mixing device.

The control system 158 is configured to control the configuration of the second valve 155, for example changing the state of fluid communication between the second fluid source 150A and other elements. In certain embodiments the controls on the applicator 156 control fluid release from the second fluid source 150A (e.g., via the second valve 155).

FIG. 30 illustrates that the first fluid source 160A, and/or the second fluid source 160B, and/or a third fluid source 160C are in fluid communication with the SDS 101F. The first and/or second and/or third fluid sources 160A and/or 160B and/or 160C are optionally pressurized, such as pressurized cartridges. The first fluid source 160A is in fluid communication with the pump 161, the first valve 162, the fourth valve 163, the venturi 164, the delivery conduit 165, and the applicator 132, similar to the fluid from the fluid source 140 as shown in FIG. 28 (with the second valve 143 in the SDS 101D in FIG. 28 being the fourth valve 163 in the SDS 101F in FIG. 30). The second valve 166 is in a fixed or variable configuration to be in fluid communication with the elements as described for the SDS 101E shown in FIG. 29 (with the third valve 153 in the SDS 101E in FIG. 29 being the fourth valve 163 in the SDS 101F in FIG. 30). The third valve 169 is in a fixed or variable configuration to place the third fluid source 160C in fluid communication with the pump 161 and/or the fourth valve 163 and/or the flow channel 167 of the SDS 101F between the fourth valve 163 and the venturi 164, and/or the delivery conduit 165, and/or the applicator 132, and/or directly with the treatment site 157.

The control system 168 is configured to control the configuration of the third valve 169, for example changing the state of fluid communication between the third fluid source 160C and other elements.

FIG. 31 illustrates SDS 101G such that the second fluid source 160B is in fluid communication with the pump 133, for example, through the second valve 171. The second valve 171 has no configurations in which the second fluid source 160B is placed in direct fluid communication with the third valve 172 and/or the flow channel of the SDS 101G between the third valve 172 and the venturi 164, and/or the delivery conduit 164A, and/or the applicator 132, and/or directly with the treatment site 157. The pump 133 is the mixing device.

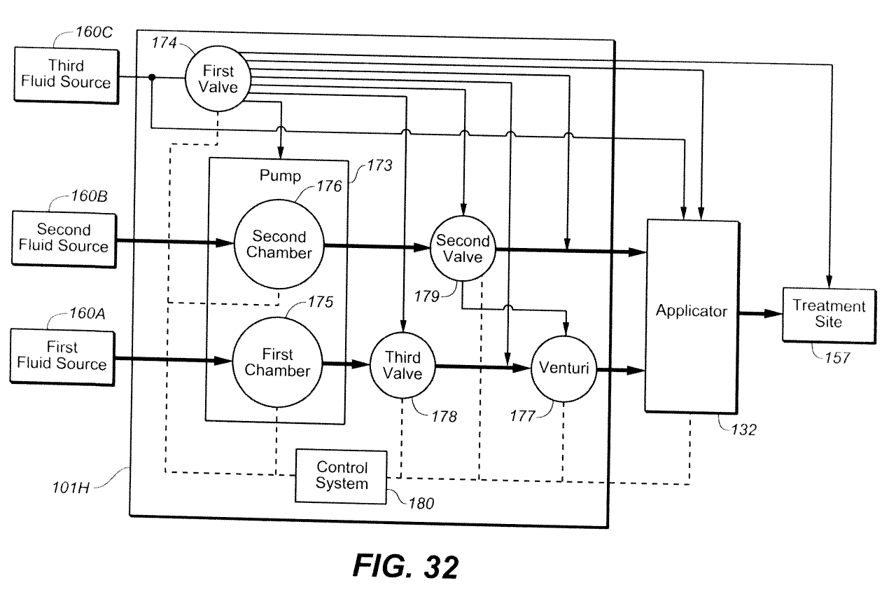

FIG. 32 is a cleaning system that illustrates that the first fluid source 160A and/or the second fluid source 160B are in direct fluid communication with the pump 173. The third fluid source 160C is in fluid communication with the first valve 174. All fluid sources are in direct fluid communication, or fluid communication via a valve, with each other. The first fluid source 160A and/or the second fluid source 160B and/or the third fluid source 160C contain individual fluids (e.g., photosensitizer solutions) that is mixed prior to, and/or concurrent with, and/or after, delivery to the treatment site 157.

The pump 173 has more than one chamber, for example a first chamber 175 and a second chamber 176. The first chamber 175 is in fluid communication with the first fluid source 160A. The second chamber 176 is in fluid communication with the second fluid source 160B. The first chamber 175 is in direct fluid communication or insulated from direct fluid communication with the second chamber 176. In certain embodiments the first chamber 175 is controllably mixed (see control system 180) with the second chamber 176.

The first chamber 175 is in fluid communication with the third valve 178. The third valve 178 is in fluid communication with the venturi 177. The venturi 177 is in fluid communication with the applicator 132, through for example the delivery conduit (not shown).

The first chamber 175 is in fluid communication with the third valve 178. The third valve 178 is in fluid communication with the venturi 177. The second valve 179 is optionally in fluid communication with the venturi 177. The venturi 177 is in fluid communication with the applicator 132, through for example the delivery conduit (not shown).

The third fluid source 160C is in direct fluid communication with the first valve 174 and/or the applicator 132. The first valve 174 is in a fixed or a variable configuration to place the third fluid source 160C in fluid communication with the pump 173 and/or the second valve 179, and/or third valve 178, and/or the flow channel of the SDS 101H between the third valve 178 and the venturi 177, and/or the flow channel of the SDS 101H between the second valve 179 and the applicator 132, and/or the applicator 132, and/or directly with the treatment site 157. Any of the aforementioned elements is optionally the mixing device.

The fluids from the first, second and third fluid sources 160A to 160C are optionally mixed in any combination at the pump 173, and/or the applicator 132, and or the flow channel, and/or the venturi 172, and/or the treatment site 157. All of the above are subject to control system 180.

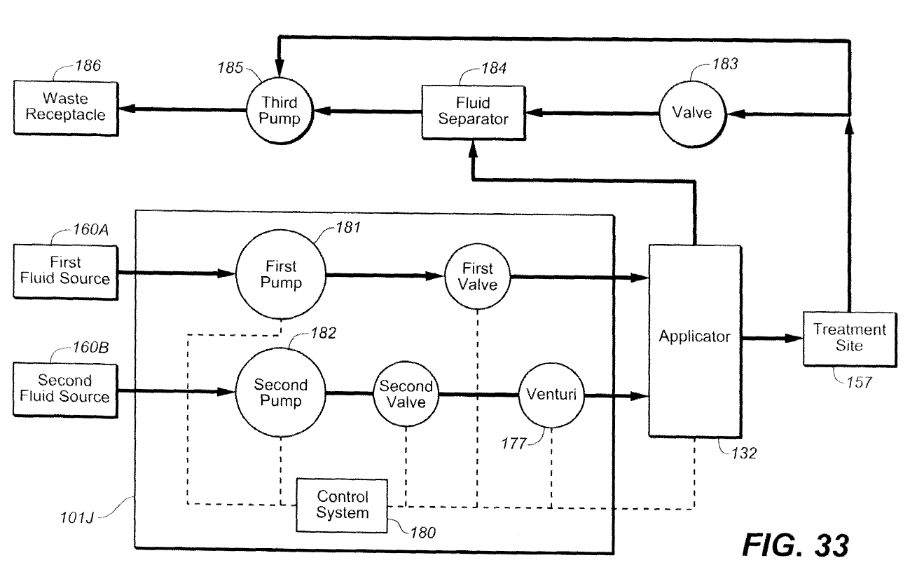

FIG. 33 illustrates an SDS 101J that has a first pump 181 and a second pump 182. The first pump 181 is in fluid communication with the first fluid source 160A, for example through a valve 160B (not shown). The first pump 181 is not in direct fluid communication with the second fluid source 160B. The second pump 182 is in fluid communication with the second fluid source 160B, for example through a valve (not shown). The second pump 182 is insulated from direct fluid communication with the first fluid source 160A.

The fluids from the first and second fluid sources 160A and 160B are mixed in any combination at the applicator 132, and/or the flow channel, including the delivery conduit, and/or the venturi 177, and/or the treatment site 157 (as shown).

In the schematic FIGS. 28-33, the solid lines are flow paths or flow channels, such as lumen. In certain embodiments the flow channels act as mixing devices, for example where two flows merge. The phantom lines are data communication paths or channels, such as wires, wireless communication pathways, processor channels, pneumatic conduits, hydraulic conduits, mechanical linkages, or combinations thereof and communication is optionally bi-directional along these paths. Arrows illustrate the flow direction; however, any flow path is optionally bidirectional. The pumps, and/or valves, and/or applicators are configured to be manually or automatically controllable. The fluid delivery systems and/or cleaning systems are configured to deliver any combination of fluids available from the fluid sources in any ratio. These combinations and ratios vary and/or stay constant during operation. The SDS 101A to 101H and 101J describe embodiments that have sub-elements such as the pumps (141, 161), one or more valves, the mixing device, or combinations thereof. The sub-elements of the SDS 101A to 101J are optionally physically attached to any or all other sub-elements. The SDS optionally has no container or case. The sub-elements of the SDS 101ff are optionally physically detached to all other sub-elements. The first, and/or second, and/or third fluid sources 160A, 160B, and/or 160C, and/or 160D are optionally an integral or fixedly or removably attached element (e.g., a multi-chamber reservoir), and/or separate elements (e.g., the cartridge 120 and the fluid intake conduit 117). In the schematic FIGS. 32 and 33, the fluids are optionally delivered in any mixed to non-mixed ratio. The mixed to non-mixed ratio optionally varies and/or stays constant during use.

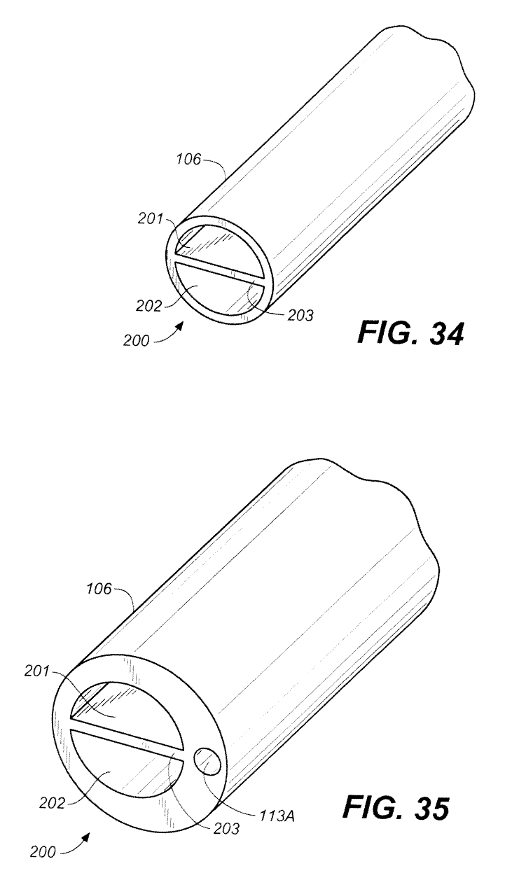

FIG. 34 illustrates that all or a portion of the length of the delivery conduit 106 has multiple channels 201 and 202 that are separated by delivery conduit septa 203. For example, the delivery conduit 106 has a first channel 201 that is fluidly isolated from a second channel 202 by the delivery conduit septum 203. Distinct fluids (e.g., photosensitizer solution, water, other fluids listed herein) flow through the distinct channels 201 and 202. The distinct fluids are mixed at the end of the delivery conduit 106, and/or in the applicator 132, and/or after being delivered by the applicator to the treatment site 157, or the distinct fluids remain unmixed.

FIG. 35 illustrates that the delivery conduit 106 is fixedly attached to or integral with an energy transport device, such as a light source 113A. In certain embodiments the light source 113A is an optical fiber embedded, molded into, or slid along a channel in the wall of the delivery conduit 106. In certain embodiments the wall of the delivery conduit 106 is thicker in the area of the light source 113A compared to the remainder of the wall. In certain embodiments the light source 113A is substantially adjacent to the end of the delivery conduit septum 203. In certain embodiments the light source 113A, such as an optical fiber, extends beyond the fluid outlet. In certain embodiments the light source, for example the distal end of the light source, has a diffuser. In certain embodiments the shape of the first channel 201 and second channel 202, the delivery conduit septum 203, and the light source 113A are configured to allow the wall of the delivery tube to be substantially uniform in thickness.

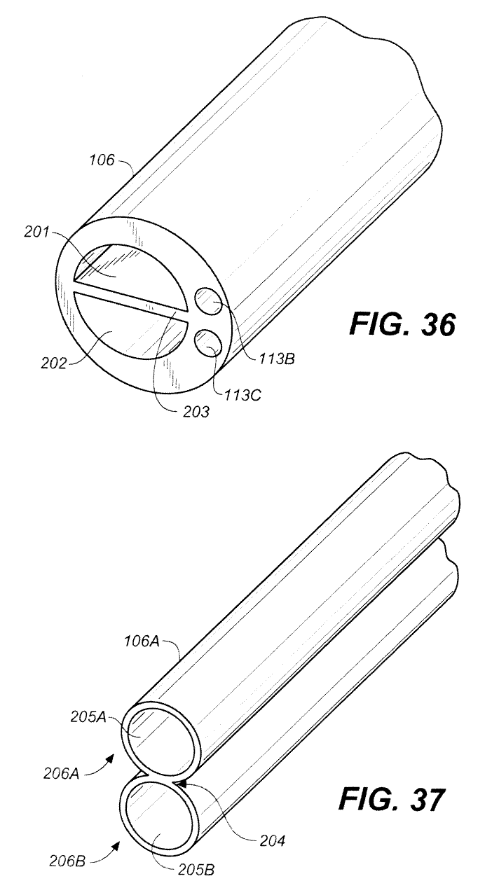

FIG. 36 illustrates that the delivery conduit 106 is fixedly attached to or integral with first and second energy transport devices, such as first and second light sources 113B and 113C. In certain embodiments the light sources 113B and 113C are substantially adjacent to the end of the delivery conduit septum 203.

FIG. 37 illustrates that the delivery conduit 106A has multiple channels that are distinct conduits that are attached or unattached to each other. For example, the first channel 205A is attached 204 to the second channel 205B. The first channel 205A and the second channel 205B are each distinct conduits each having a fluid outlet 206A and 206B.

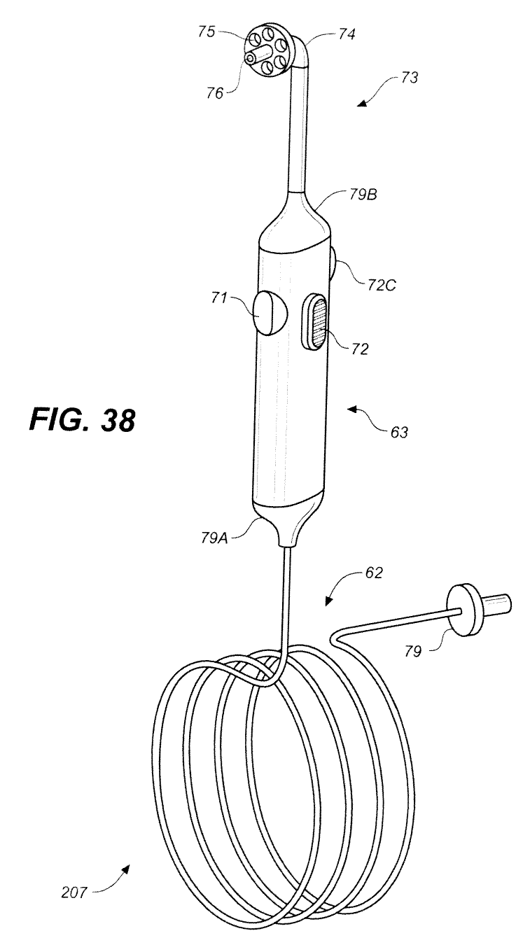

FIG. 38 illustrates that the controls on a portion of the cleaning system 63 include one or more of power controls 72C and fluid controls 71 on the applicator 63. This cleaning system also has light controls 72. In certain embodiments the controls 71, 72 and 72C are on the SDS (not shown) and/or on the applicator 63, and/or on the delivery conduit 62, and/or on the reservoir (not shown) and/or on the cartridge. The delivery conduit 62 has a connector at a first and 79 and/or a second end 79A.

The applicator 63 is integral with or fixedly attached to the delivery conduit 62. In certain embodiments the applicator 63 and delivery conduit 62 are releasably attachable to the cleaning system and/or the fluid source (not shown). The applicator 63 and/or delivery conduit 62 are optionally reused between different cleaning systems and/or fluid sources. The applicator 63 optionally has a replaceable tip 73 (e.g., at connector 79B).

In certain embodiments the cleaning system has measurement devices and/or indicators (e.g., icons that are backlit when the function they refer to is active, or conditions they refer to are being met) that display the state of the cleaning system and the operation of the cleaning system. For example, the cleaning system optionally displays the temperature of the fluids, and/or the pressure of the cartridge, and/or the fluid flow rate, and/or the fluid flow frequency, and/or the light energy intensity, and/or the light energy frequency, and/or the light energy strobe frequency and/or pattern and/or other characteristic of the energy emission profile.

The fluid flow is optionally delivered at a specific frequency when the cleaning system delivers slugs of fluid of a specific slug volume and a slug delay between each slug. The slugs have a slug diameter from about 0.005 in (0.125 mm) to about 0.16 in (4 mm) for example about 0.06 in (1.5 mm). The slugs have a slug volume from about 0.05 ml to about 2 ml, for example about 0.1 ml, or about 0.25 ml, or about 0.5 ml, or about 0.75 ml, or about 1 ml. The slug delay is from about 0.01 sec to about 60 sec, for example about 0.1 sec, or about 0.2 sec, or about 0.5 sec, or about 1 sec, or about 15 sec, or about 30 sec, or about 60 sec.

FIG. 38 illustrates that the delivery conduit 62 is configured to be releasably attached to the SDS. The fluid controls 71 are on the applicator 63. The fluid controls 71 are knobs, switches, dials, levers, toggles, tabs, buttons, slides, accelerometers, fluid or contact pressure sensors, other rotating switches, other translating switches, or combinations thereof. Separate fluid controls 71, 72, and 72C are used to rough-tune and fine-tune the fluid flow characteristics.

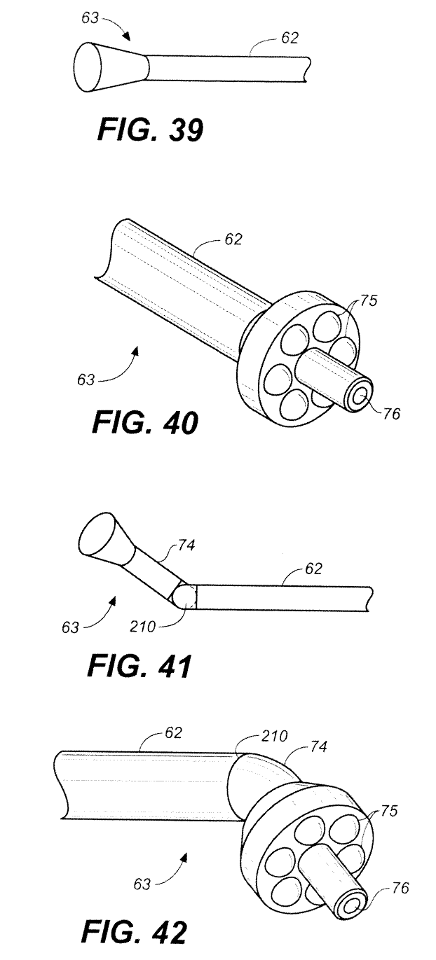

FIGS. 39 and 40 illustrate that the delivery conduit 62 is straight. The applicator 63 has a conical configuration. The applicator 63 has optionally one or more light sources 75. The light sources 75 are optionally positioned equiradially around the center of the applicator 63 and/or optionally equidistant from the other light sources 75. The fluid outlet 76 is in the center of the applicator 63. The fluid outlet 76 extends beyond the distal surface of the applicator 63.

FIG. 41 illustrates that the delivery conduit 62 has a neck 74 that is attached to the applicator 63. The neck 74 is rotatably attached to the remainder of the delivery conduit 62 by a hinge 210. The hinge 210 is configured to rotate in one and/or two-dimensions. For example, the hinge 210 is a ball-in-socket joint.

FIG. 42 illustrates that the neck 74 is optionally rotatably attached to the remainder of the delivery conduit 62 by a hinge 210. The applicator 63 has one or more light sources 75. The light sources 75 are optionally positioned equiradially around the center of the applicator 63 and/or equidistant from the other light sources 75A. The fluid outlet 76 is in the center of the applicator 63. The fluid outlet 76 extends from the applicator 63.

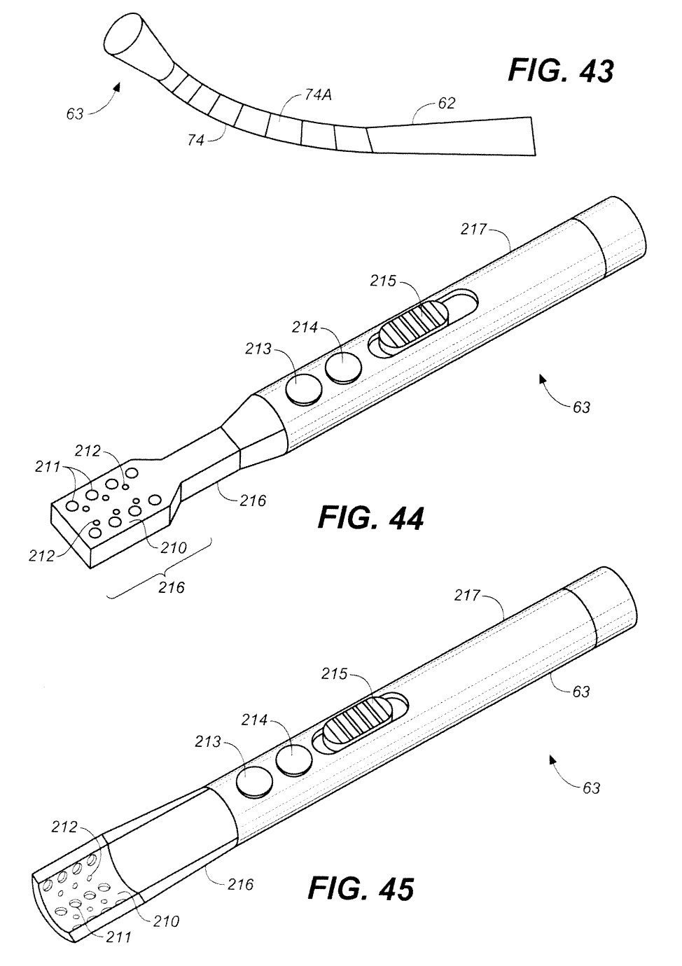

FIG. 43 illustrates that the neck 74 is segmented. In certain embodiments the neck is a reinforced coil. The neck 74 optionally has neck segments 74A. Each neck segment 74A is rotatably attached to the adjacent neck segments and/or delivery conduit 62. In certain embodiments the neck segments 74A are configured to rotate in one and/or two-dimensions. In certain embodiments the neck segments 74A have ball-in-socket joints. In certain embodiments the neck segments 74A are reinforced by a coil.

FIG. 44 illustrates that the applicator 63 has a flat configuration with a face 210. The applicator 63 has transducers 211, for example light sources 211, on the face 210. The applicator 63 has fluid outlets 212 on the face 210. Multiple sides (not shown, but is opposite side from that shown) of the applicator 63 have faces 210 that optionally have light sources 211 and/or fluid outlets 212. The applicator 63 has the light control 213, and/or the fluid control 214 and/or the other control 215. In certain embodiments the tip 216 is releasably attached to the body 217 so that different tips are used. In certain embodiments these tips have the same or different transducers and therefore different energy emission profiles, including emitting energy of different frequencies.

A disposable cover (e.g., transparent to the energy from the light source) is optionally used to cover a portion (e.g., the tip, the tip and controls), and/or the entire applicator. The cover is flexible, rigid, or combinations thereof (e.g., flexible polymer and/or injection molded polymer). In certain embodiments the applicator and/or cover are mechanically and/or magnetically aligned and/or linked together, for example locating and/or retaining the cover on the applicator (e.g., protrusions, ridges, elastic bands, connectors). The applicator and/or the cover release the mechanical and/or magnetic linkage between the cover and applicator, for example allowing for the removal of the cover from the applicator. In certain embodiments the cover forms a fluid resistant or fluid tight seal with the applicator, for example through a connector or through the use of a reversibly expandable component such as an elastic ring. The cover is optionally packaged in an individual container. The individual container keeps the cover clean and/or sterile (e.g., sealed flexible pouch, sealed thermoformed container). In certain embodiments the packaging is constructed to aid in the placement of the cover over or onto the applicator in a way that prevents contact between the user and the cover. For example, the package opens to a defined position that exposes a connector and the opening to the connector, but still leaves a portion of the cover covered by the packaging so that it is possible for the user to install the cover onto the applicator without directly contacting the cover. The user inserts the applicator into the cover until the connector is engaged in a locked position, and then removes the now covered applicator from the cover packaging. In certain embodiments the cover and/or the packaging contain separate and/or fixedly and/or removably attached components configured to aid with the placement and or retention of the cover. For example, a cover has an entrance surrounded by an elastic ring and an insertion aid (e.g., a rigid or semi-rigid ring with a groove) that is separate from the elastic ring placed in the cover entrance so that the elastic element is aligned with the groove and maintained in an open configuration such that the applicator is inserted through the elastic ring and into the cover with little or no resistance. The separate insertion aid is then removed from the cover entrance allowing the elastic element to constrict onto the applicator and provide a force that retains the cover on the applicator.

In certain embodiments the applicator 63 has bristles extending therefrom. The bristles are transparent, translucent, or transmissive.

FIG. 45 illustrates that the face 210 is curved. The face 210 has a constant or continuously changing radius of curvature along substantially or completely the entire face 210.

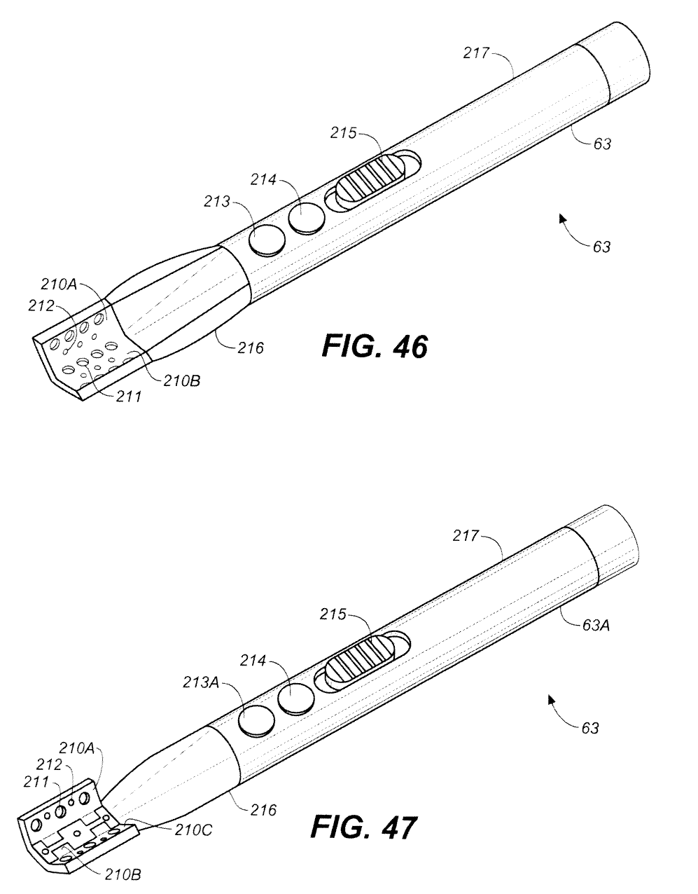

FIG. 46 illustrates an applicator 63 wherein the face 210 is optionally configured as an angled or “V”-shape. FIG. 47 illustrates that multiple faces 210 are rotatably attached to a fixed face. A first face 210A, and a third face 210C are rotatably attached by hinges to a second face 210B that is fixed. A first face and a second face are rotatably attached by a hinge to each other to form an adjustable “V” shape.

In certain embodiments the light controls 215, and/or the fluid controls 214, and/or the other controls 213A are bi-modal (i.e., two settings) and/or multi-modal (i.e., two or more settings), and/or modal (i.e., having definite settings) and/or analog (i.e., substantially infinitely variable within the range of settings). The light controls 215, and/or the fluid controls 214, and/or the other controls 213A are knobs, switches, dials, levers, toggles, tabs, buttons, slides, accelerometers, fluid or contact pressure sensors, other rotating switches, other translating switches, or combinations thereof.

The applicators 63 shown in FIGS. 39-47 have handles, for example instead of delivery conduits 62. The applicators 63 shown in FIGS. 39-47 are optionally absent of fluid sources. In certain embodiments the applicators shown in FIGS. 39-47 have a pressurized and/or non-pressurized cartridge of sensitizer solution, for example contained in the handle, and/or the handle is connected to the SDS through a delivery conduit. In certain embodiments the cartridge is inaccessible, requiring disposal of the device when the cartridge contents are consumed. In certain embodiments the cartridge is replaceable or refillable, for example from a larger pressurized or non-pressurized container.

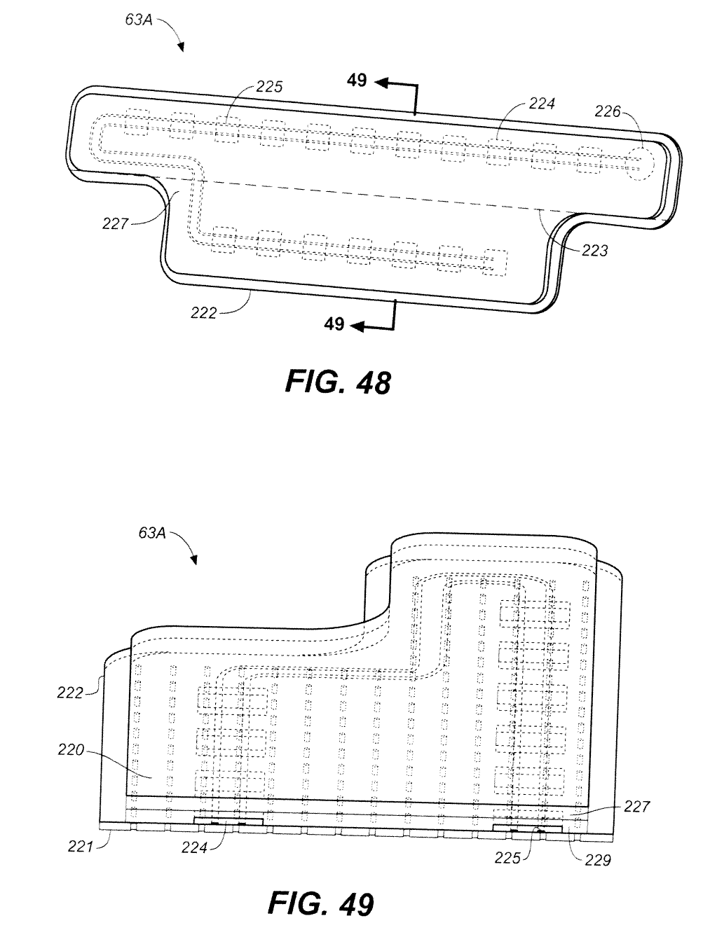

FIGS. 48 and 49 illustrate that the applicator 63A has a treatment side 220 and an outside 221. The applicator 63A is a sheet. The applicator 63A is flexible. The applicator 63A is configured to conform to the shape of the surface onto which the applicator 63A is applied. The applicator has a flexible backing sheet 222 having electrical connection 225. The outline (e.g., pattern of the circumferential edge) and shape (e.g., curvature) of the applicator are pre-configured for a specific treatment site, for example upper teeth, lower teeth, tongue, specific skin area (e.g., bridge of the nose, around the mouth, a “whole face” mask), and/or can come in sizes (e.g., small, medium, large). As shown in FIG. 48, the applicator 63A is shaped to cover the labial and lingual surfaces of the upper or lower teeth, for example, by folding the applicator at a fold line 223.

The transducers 224 are mounted to the treatment side 220 of the backing sheet 222 (as shown). The transducers are optionally mounted on the outside 221 surface of the backing sheet 222 (similar to those shown in FIG. 51). The backing sheet 222 allows transmission of the energy from the transducers 224.

One or more connections, for example traces 225, connect the transducers 224 to each other and/or to an energy (e.g., electrical and/or data and/or light and/or control and/or power) source (e.g., power cell 226). The traces 225 are optionally fiber optics. In certain embodiments the traces 225 are wires. In certain embodiments the traces 225 are embedded conductors.

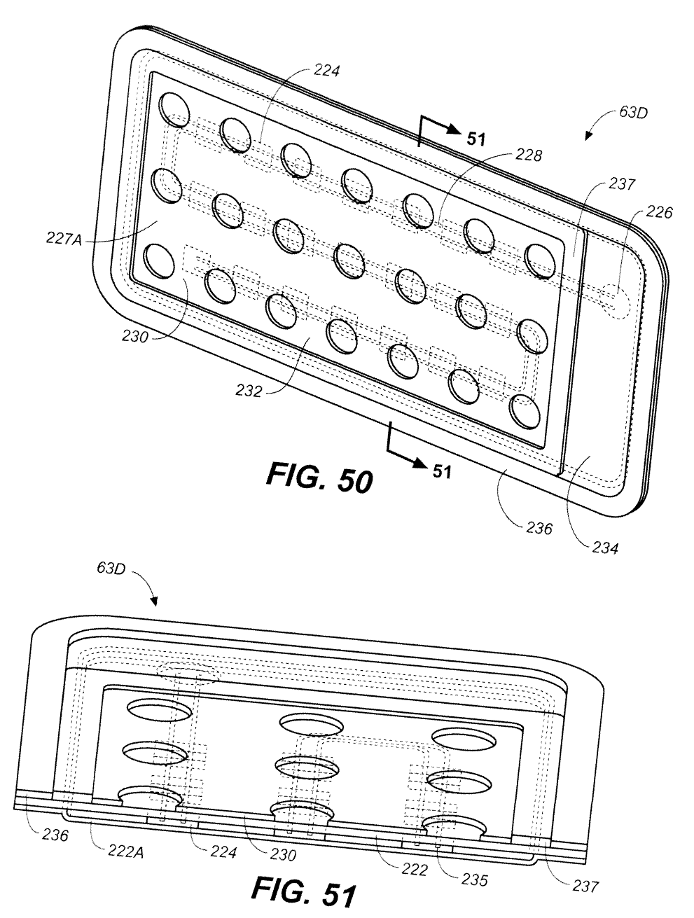

In certain embodiments a flexible illuminator (i.e., an illuminator sheet 234) has the transducers, the connections, and the energy sources attached, for example in arrangements similar to those illustrated in FIGS. 48 and 50, to a flexible backing sheet 222. FIGS. 50, 51, 52, 53, and 54 illustrate that in certain embodiments the cleaning system has a flexible illuminator separate from the applicator. The applicator backing sheet 222 is transmissive and/or conductive to the energy emitted by the illuminator sheet transducers 224. For example, the applicator backing sheet 222 is transparent and the transducers emit visible light. The illuminator sheet 234 is sized to fully cover the applicator. The entire surface of the applicator 63D is exposed to the energy of the illuminator sheet 234. The illuminator sheet 234 is separatably attached to the applicator 63D.

In certain embodiments the applicator 63ff have all or a portion of the applicator's surface designed (e.g., textured, dimpled, porous, coated, perforated) to aid in the retention of the sensitizer solution. All or any portion of the applicator's surface, for example the surface of the backing sheet 222, has features (e.g., depressions (e.g., channels, grooves, dimples), protrusions (e.g., bumps, ridges), perforations), for example, to improve the mobility and/or mixing of the sensitizer solution compounds with each other or with compounds at the treatment site (e.g., saliva, sweat, blood plasma, blood serum, interstitial fluid, mucous, urine, lymph, vaginal fluids, irrigation fluids (e.g., water, ringers lactate, saline, etc.), hereafter bodily fluids.

In certain embodiments the applicator 63ff (aka 63A, 63B, 63C, 63D, or combinations thereof) has one or more areas covered by one or more fluid permeable materials, for example, porous materials. The porous material is optionally the porous structure of a natural and/or artificial material (e.g., open cell foam, polyvinyl alcohol (PVA), polyurethane, polyvinyl chloride (PVC), polyolefin, polystyrene), woven polymer and/or fabric (e.g., woven cotton, polyester, silk), bonded and or non-bonded polymer and/or fabric matting, paper, polytetrafluoroethylene (PTFE), expanded polytetrafluoroethylene (EPTFE)), a fluid impermeable material (e.g., metal foil, polymer, closed cell foam (e.g., polyvinyl alcohol (PVA), polyurethane, polyvinyl chloride (PVC), polyolefin, polystyrene), polytetrafluoroethylene (PTFE), expanded polytetrafluoroethylene (EPTFE)) or combinations thereof. The porous material forms a porous layer. The applicator 63ff optionally has a porous layer on the treatment side 220 surface. The applicator 63ff has an impermeable polymer layer on the outside surface 221. The porous material has pores and/or fibers. The pores are air-filled. A portion of the volume of the porous structure contains air. From about 5% to about 95% of the porous material volume is air. In certain embodiments the applicator 63ff has one or more permeable regions made impermeable. For example, the applicator is coated in whole or part with an impermeable coating such as Parylene.

In certain embodiments the applicator 63ff is made from and/or coated with an absorbent 229 (e.g., a water-absorbing and/or bodily fluid-absorbing material). The absorbent 229 absorbs water and/or bodily fluid and increases in volume. In certain embodiments the absorbent 229 is a layer applied to the treatment side of the backing sheet.

The absorbent 229 is a natural or artificial material (e.g., cotton, hydrogel) and conforms to the treatment site and/or the absorbent's surroundings. The expansion in volume of the absorbent 229 can result, for example, in the shape of the applicator 63ff conforming to the structures that the applicator is applied over. In a certain embodiment the backing sheet 222 folds over the upper and/or lower teeth and/or gums.

In certain embodiments the backing sheet 222 has an absorbent layer 229, for example a hydrogel, on the treatment side of the backing sheet. The absorbent layer 229 has a layer of sensitizer solution 227, for example in the form of a gel, distributed over the top of the hydrogel.

When the applicator 63ff is applied to a treatment site where fluids are present, for example over the upper and/or lower teeth and gums in the oral cavity, fluids, for example water from saliva, move through the porous material and/or perforated backing sheet 222 and into the polymer matrix of the hydrogel. The hydrogel expands, for example as the hydrogel absorbs water. This expansion pushes the sensitizer gel toward the treatment site (e.g., teeth and gums) and the backing sheet toward the lips.

In certain embodiments the hydrogel prevents the fluid in the treatment site, for example saliva, from mixing with, diluting, and/or washing out the sensitizer solution from the treatment site. In certain embodiments a hydrogel layer is a seal. For example, the perimeter of the applicator includes the hydrogel. The hydrogel surrounds the region of the applicator 63ff coated with sensitizer solution.

The perforations have a diameter from about 0.001 in. to about 0.1 in. More narrowly, the perforations have a diameter from about 0.005 in. to about 0.01 in., or from about 0.01 in. to about 0.02 in., or from about 0.02 in. to about 0.04 in., or from about 0.04 in. to about 0.06 in., or from about 0.06 in. to about 0.08 in., or from about 0.08 in. to about 0.1 in. The perforations are uniform in diameter. The diameter of the perforations does vary.

FIG. 49 is a rotated view of the cross section 49-49 of FIG. 48. The applicator 63A of FIGS. 48 and 49 includes a sensitized solution layer 227 and an absorbent layer 229.

FIGS. 50, 51, and 52 illustrate that the sensitizer solution has two or more different components, for example sensitizer solution part A 230 and sensitizer solution part B 231. FIG. 51 is a rotated view of the cross section 51-51 of FIG. 50.

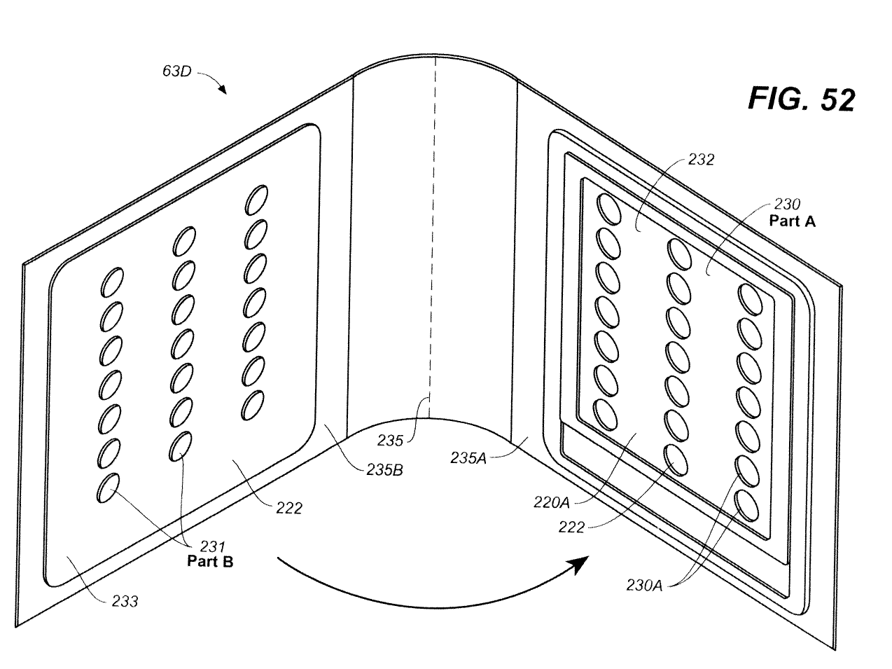

The sensitizer solution part A 230 and the sensitizer solution part B 231 are separate until use. As shown in FIG. 52, sensitizer solution part A 230 is on a backing sheet 222. Sensitizer solution part B 231 is on a backing sheet 222. The two backing sheets are integral and/or removably attached to the alignment aids 235A and 235B.

In certain embodiments part A 230 and part B 231 react with each other and/or with fluids in the treatment site and/or with the tissue surface in the treatment site. In certain embodiments the sensitizer components 230 and 231 are distributed in a complementary pattern. For example, when part A is placed in contact with part B, the surface of the applicator 232 is completely or substantially covered with a component of the sensitizer solution. Part A is distributed on to the treatment side 227A of a backing sheet 222. Part B is removably distributed onto a disposable backing sheet 222.

In certain embodiments the multiple component sensitizer solution cleaning system has alignment aids 235A 235B to ensure that the sensitizer solution components are brought together in the proper way. For example, the outside surfaces of the backing sheets of part A and part B are removably attached to the alignment aids 235A and 235B, for example a flexible sheet with a fold line 235. The fold line 235 is optionally perforated and/or hinged. The alignment aid rotates about the fold line.

FIGS. 50, 51, 52, 53, and 54 illustrate that the applicators 63D and 63E are optionally configured to isolate the treatment site. The applicators 63D and 63E confine the sensitizer solution and bodily fluids to the treatment site. In certain embodiments the applicator surface is coated with an adhesive. The adhesive aids in attachment of components (e.g., sensitizer solution, seal, transducers) to the applicator. In certain embodiments the adhesive attaches the applicator to the adjacent site, for example the skin surrounding a wound, other skin, mucous membrane, and combinations thereof. In certain embodiments the adhesive is biocompatible.

In certain embodiments the adhesive 236 is around the entire perimeter of the applicator 63D. The adhesive provides a force to hold the seal 237 in compression against the adjacent site. The inner edge of the seal 237 defines the treatment site. In certain embodiments the seal 237 is compressible, for example a polymer foam and/or hydrogel. In certain embodiments the seal 237 is flexible, for example a rubber lip seal. In certain embodiments a hydrogel seal 237 absorbs fluid, for example, before the fluid enters the area exposed to the sensitizer solution. The hydrogel seal contains and/or is coated with an adhesive, for example a bioadhesive, for example, to provide a better seal.

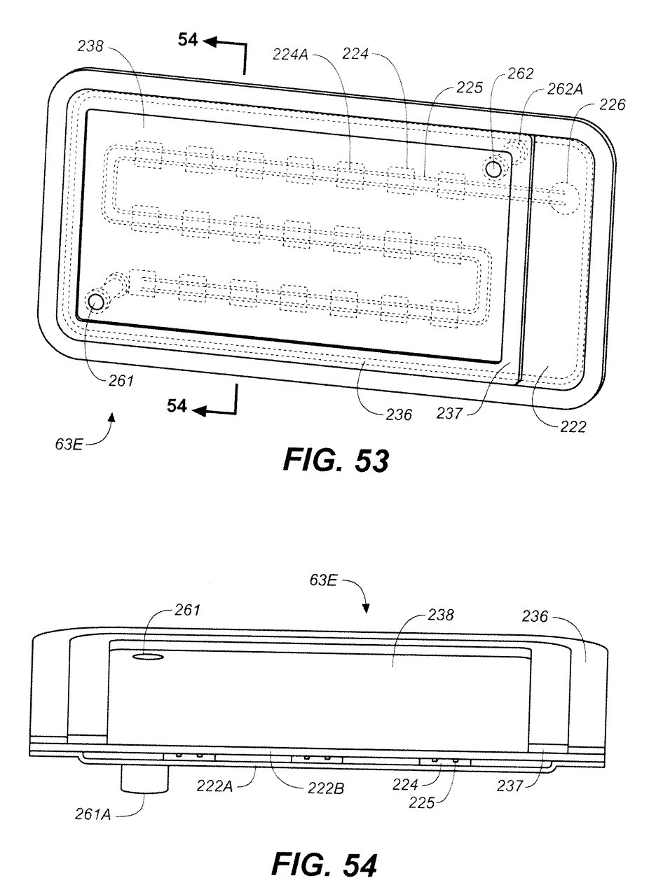

FIGS. 53 and 54 illustrate that the fluid delivery system (not shown), for example the SDS, is attached to the applicator 63E. The fluid delivery system can connect to the applicator 63E through fluid connections 261A and 262A.

The backing sheet 222 is transparent or transmissive to the energy of the transducers 224. The transducers 224 are on the outside of the backing sheet 222. The external surface of transducers 224 and electrical connections 225 are covered by a backing sheet 222. The seal 237 has a sensitizer solution space 238. During use, sensitizer solution (not shown) flows over the treatment site by entering through the fluid inlet 261, flowing across the sensitizer solution space 238, and exiting through the fluid outlet 262.

In certain embodiments the applicator has a configuration customized for a particular application and/or user. The applicator is cut with a scissors into a configuration.

The applicator 63E has an internal 226 or external power source, internal or external controls, and internal transducers 224 in electrical communication 225 with each other. In certain embodiments the applicator 63E has a separation layer (not shown), for example a layer located between the backing sheet 222 and the sensitizer solution. In certain embodiments the applicator is used in combination with a photosensitizer to treat a wound and/or infection, for example as taught by U.S. Pat. No. 6,251,127 by Biel, which is hereby incorporated by reference in its entirety.

In certain embodiments the sensitizer solution is applied to the treatment site (e.g., by finger, syringe, toothbrush, sterile or non-sterile gauze, cotton swab). The flexible applicator 63ff is applied over the sensitizer solution-treated area. The applicator can keep the solution in place and/or protect the solution from the environment (e.g., saliva, sweat, blood, mechanical motion of other structures (e.g., tongue, cheek, scratching, clothes)). In certain embodiments the sensitizer solution, for example a gel, is applied by the user directly to a portion, all, or substantially all of one or both surfaces of the applicator 63ff. In certain embodiments the sensitizer solution, for example in the form of a liquid, is applied to an absorptive applicator to fill a portion, all, or substantially all of the air filled volume of the applicator 63ff (i.e., make it saturated). The applicator 63ff has features that indicate the area where the sensitizer solution should be applied, for example, printed lines and or text, and/or a texture applied to the area to be covered.

In certain embodiments the applicator 63A 63D is designed to limit the area that contains sensitizer solution. For example, an absorptive material is applied to a portion of the surface of the applicator 63A 63D. The sensitizer solution is applied to the absorptive material-applied portion of the surface of the applicator 63A 63D. The sensitizer solution treated (e.g. coated, saturated) applicator 63A 63D is applied to the treatment site.

In certain embodiments one or more areas of the applicator 63ff are pre-treated (e.g., pre-coated and/or pre-saturated) with sensitizer solution 230. For example, none of the internal volume (i.e., the volume not occupied by the material of the applicator 63ff) of the porous material is filled with sensitizer solution 230, or about 10%, or about 20%, or about 30%, or about 40%, or about 50%, or about 60%, or about 70%, or about 80%, or about 90% or about 100% of the internal volume of the porous material is filled with sensitizer solution. In certain embodiments the pre-coated area(s) cover a portion, for example between 20% and 100%, or about 20%, or about 30%, or about 40%, or about 50%, or about 60%, or about 70%, or about 80%, or about 90% or about 100% of the surface of the applicator.

The treated area(s) has a treated shape. In certain embodiments the treated shape is a regular shape (e.g., rectangle, square, circle, rhomboid, triangle, etc.), an irregular shape (e.g., any shape other than a regular shape enclosed by a single path). In certain embodiments a first treated area has the same and/or a different treated shape as a second treated area.

In certain embodiments the sensitizer solution (e.g.) 227, 230, 231 in each region and between regions has a uniform or variable quantity and/or composition of sensitizer. For example, each region has a different sensitizer solution composition (e.g., type of photosensitizer, concentration of a particular photosensitizer, mixture of photosensitizers and/or additives and/or reactive components) and/or a different quantity of sensitizer solution pre-coated onto the region and/or pre-saturated into the region. In certain embodiments the interior of the applicator and the surface is filled and/or coated with different quantities and/or different compositions of sensitizer solution. In certain embodiments the quantity and/or composition of the sensitizer solution varies discretely and/or continuously over one or more regions, or over the entire area coated and/or saturated with sensitizer solution. Variations of quantity and/or composition of the sensitizer directly correlate to the location (e.g., horizontal and/or vertical) of a region of the applicator.

In certain embodiments the applicator 63ff has a disposable cover sheet (not shown). The backing sheet 222 and/or cover sheet prevents the sensitizer solution from being rubbed off and/or contaminated during storage and/or use.

In certain embodiments the sensitizer solution has two or more parts, for example part A and part B. Parts A and B are different in composition and contain compounds that undergo a chemical reaction when parts A and B are brought into contact.

In certain embodiments multiple sensitizer solution compounds (e.g., reactive components) are on the same applicator but applied and packaged in a way that limits or prevents the contact between the sensitizer solution compounds. In certain embodiments sensitizer solution compounds (e.g., reactive components) are arranged in an alternating pattern of individual regions. The regions optionally touch each other, for example limiting the amount of contact between regions and thus reaction. The regions are optionally separated by some amount of space. For example, the applicator has a matrix of areas (e.g., dots) or lines.

Sensitizer solution compounds (e.g., reactive compounds) are optionally separated by a temporary barrier. For example, the porous volume of the applicator is saturated with a first reactive compound (e.g., part A) and then coated by a separation layer. The separation layer is then coated with a second reactive compound (e.g., part B) of the sensitizer solution. The separation layer keeps parts A and B separate until the separation layer is activated. When activated the separation layer becomes permeable to part A and/or part B. Part A then reacts with part B. The separation layer is optionally activated. Activation optionally includes dissolving in, and/or expanding in fluid at the treatment site (e.g., saliva, sweat, blood plasma, blood serum, interstitial fluid, mucous, urine, lymph, vaginal fluids, irrigation fluids (e.g., water, ringers lactate, saline). In certain embodiments pores in the separation layer expand when activated. The expanded pores are permeable by part A and/or part B.

Part A is optionally applied to the applicator. The applicator can then be dried. Part B, for example in fluid form with no aqueous base, is applied to the applicator. The cleaning system optionally has more than one applicator, for example applicator A and applicator B. Applicator A has part A but no part B. Applicator B has part B but no part A.

Applicator A has a backing sheet that is over the treatment site during use. The exposed surface of the sensitizer solution on applicators A and B is covered by a disposable cover sheet that prevents the sensitizer solution from being rubbed off or contaminated during storage and/or use. The backing sheet and cover sheet of applicator B are disposable sheets. Applicator A and applicator B are optionally packaged together or separately. Applicator A and B are designed, constructed, and packaged to prevent the sensitizer solution from being rubbed off and/or contaminated during storage and/or use.

The area of applicator A that is treated with the sensitizer solution is optionally a mirror image of the area of applicator B that is treated. When applicator A is placed onto applicator B, the two areas of sensitizer solution substantially align with and cover each other. The applicator is optionally configured to cover the teeth and gums.

The corresponding areas of the applicators that align with the teeth and gums optionally include a teeth region and a gum region, respectively. The teeth region has a first sensitizer compound. The gum region has a second sensitizer compound. The first sensitizer compound optionally has a bleaching agent, and/or penetrant or a different concentration of an agent (e.g., sensitizer, peroxide, penetrant, targeting moiety) and/or a different agent (e.g., sensitizer, peroxide, penetrant, targeting moiety) than the second sensitizer compound.

Cover sheets on applicator A and applicator B are removed. The cover sheets are optionally numbered, colored, or otherwise coded, for example, to designate the order in which the cover sheets should be removed.

In certain embodiments the applicator is activated, for example, by soaking in water or heating in a microwave. The activating dissolves the separation layer or hydrates the sensitizer solution. The applicator is activated for a period of time or until a noticeable event (e.g., a color change, evolution of bubbles) occurs.

In certain embodiments the sensitizer solution is applied to the treatment site. The applicator is then applied over the sensitizer solution. In certain embodiments the applicator is treated with a solution before being applied to the treatment site. In certain embodiments a bleaching agent and/or bleaching catalyst are applied to the teeth surfaces. In certain embodiments the applicator is coated with a sensitizer solution that has no bleaching agent but has a bactericide. In certain embodiments the applicator is applied to the teeth surfaces, and/or other oral surfaces.

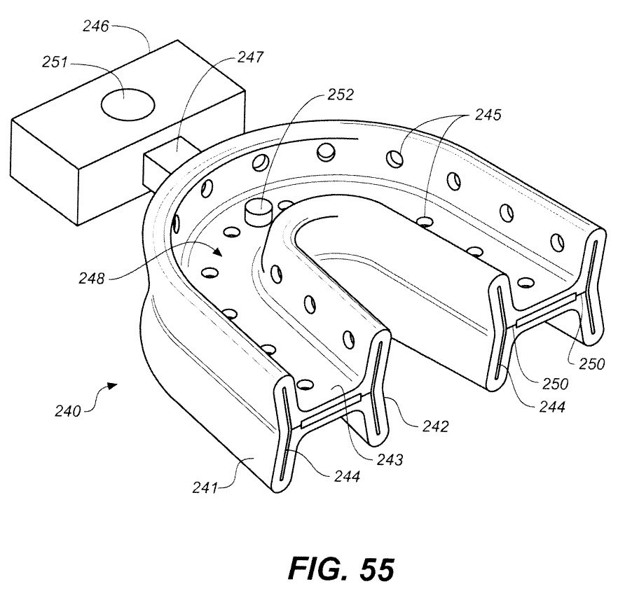

FIG. 55 illustrates that a mouthpiece 240 has a buccal sidewall 241, and/or a lingual sidewall 242, and/or a bite wafer or bite panel 243. In certain embodiments the buccal sidewall 241 and/or lingual sidewall 242 extend to only one side of the bite panel creating a single dental channel. The single dental channel is sized to fit over the upper or lower teeth.

The mouthpiece 240 is a part of the cleaning system. The mouthpiece 240 has one or more transducers 244 (having connections 250), for example light sources 245 and/or acoustic devices, and/or electrodes and/or coils and/or plates for creating electric and/or magnetic fields. The transducers 244 deliver energy to the oral cavity, for example light energy and/or acoustic energy (e.g., ultrasound), and/or create electric fields and/or magnetic fields. The transducers are configured to direct energy in any direction.

The mouthpiece 240 is in electrical communication with a power source 246. A power connector 247 attaches the mouthpiece 240 to the power source 246. The power source 246 includes one or more electrical cells (e.g., batteries) and/or a connection to an external power supply (e.g., an electrical wall outlet, an infinite bus supply). The power source 246 contains the illumination device. The power connector 247 and/or the power source 246 are removably attached to the mouthpiece 240. The power connector 247 is removably attached to the power source 246.

The mouthpiece 240 is sized to cover the upper teeth, and/or the lower teeth, as well as a portion or all of the gingiva and/or inner wall of the cheek. The mouthpiece comes in a number of standard sizes (e.g., extra small, small, medium, large, extra large). In certain embodiments the mouthpiece is customized to accommodate the hard and/or soft tissues of an individual's mouth. Such customization techniques are disclosed in U.S. Pat. No. 5,234,342.

Specific components, or the entire mouthpiece are also designed so that heating the mouthpiece, for example by immersion in hot water, makes one or more of the components of the mouthpiece pliable. Application of force to the pliable mouthpiece, for example by the user inserting the mouthpiece into his mouth and clamping his teeth down onto it, can cause deformation of the mouthpiece materials. Maintenance of this force during the period in which the mouthpiece cools makes the deformation permanent, thereby leading to a customization of the shape of the mouthpiece to the particular structures of the user's mouth.

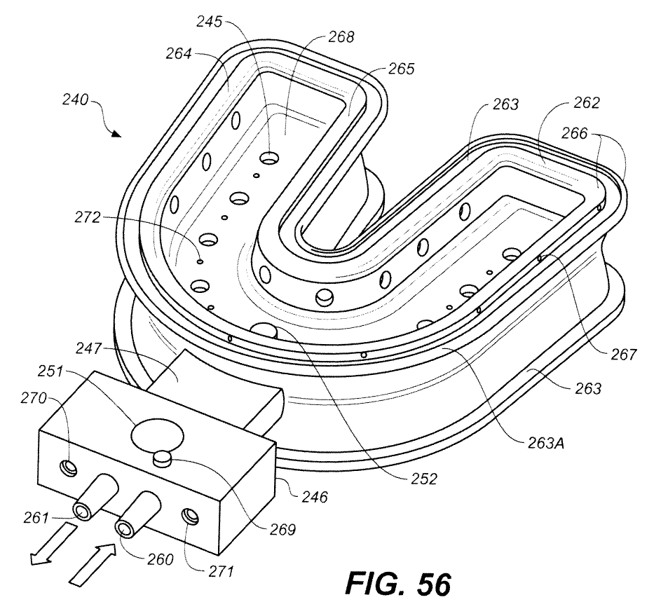

FIG. 56 illustrates that a mouthpiece has one or more fluid inlets 260 and/or a fluid outlets 261. The fluid inlets 260 and/or fluid outlets 261 are connected to one or more external fluid sources (not shown) and external vacuum sources (not shown), respectively. A fluid inlet 260 supplies a fluid, such as water and/or sensitizer solution and/or gas. The fluid inlet 260 is in fluid communication with one or more fluid ports 272 and one or more fluid sources (not shown). A fluid outlet 261 removes any fluid from in and/or around the treatment site. A fluid outlet 261 is in fluid communication with one or more vacuum ports 267 and one or more vacuum sources (not shown). The fluid inlet 260 and outlet 261 are optionally attached to a single connector (not shown). In certain embodiments the sensitizer solution is delivered and removed through a single fluid conduit, for example for tidal therapy. The solution is delivered and the energy applied. Then the vacuum is applied to the same delivery conduit, for example removing sensitizer solution, and/or saliva from the treatment site. The tidal therapy frequency and duration are optionally controlled by automatically (e.g., through sensor feedback) and/or manually. Automatic and/or manual control are optionally used to control the amount of fluid delivered, the rate of fluid delivery, the transducer's energy characteristics, the dwell time (i.e., the amount of time that the solution is in the treatment site before being removed), the fluid removal rate, the level of vacuum, and/or the time delay between cycles. Solution delivery and removal cycles are optionally for pretreatment, treatment, neutralization, rinsing, or combinations thereof. In certain embodiments the fluid source and the vacuum source are parts of a single system, for example an SDS. In certain embodiments a vacuum device, for example a suction tube, is inserted into the treatment site separately from the mouthpiece. In certain embodiments the mouthpiece 240 is constructed to deliver solutions to the treatment site and minimize the exposure of any area outside the treatment site to the solutions. The mouthpiece 240 surrounds the treatment site. The mouthpiece 240 optionally controls (e.g., direct, limit) the movement of fluids in and around the treatment site (e.g., with holes, channels, notches, grooves, protrusions). The mouthpiece optionally has endwall panels 262 that block the ends of the channels. The mouthpiece optionally has one or more sidewall panels 263 and 263A that extend away from the buccal sidewall 264 and/or lingual sidewall 265 and/or other sidewall panels 268. The sidewall panels are oriented vertically 263A, horizontally 268, or at an angle between vertical and horizontal. In certain embodiments the sidewall panels 264, 263A, and 265 are substantially perpendicular to the bite panel 268. In certain embodiments the sidewall panels 263, 264 and 265 extend from a sidewall surface at an angle that is substantially different than 90 degrees. The sidewall panels optionally have a planar structure, a curved structure, or combinations thereof. The mouthpiece has both vertical sidewall panels and horizontal sidewall panels. In certain embodiments the sidewall panels keep tissues in the surrounding area from impinging on the mouthpiece.

The sidewall panels 263 and 263A are part of a larger structure, for example a substantially horizontal sidewall panel 263 and a substantially vertical sidewall 263A extending from a buccal sidewall 264 to form a channel, for example a fluid collection channel 266. The fluid collection channel 266 acts as a barrier to any fluids entering or leaving the treatment site. The vacuum ports 267 are positioned along the channel to suck up any fluid that enters the channel. The channel concentrates (i.e., direct or focus) the vacuum around the perimeter of the treatment site. The sidewalls 263A, and 263 to 265 shield the transducers and/or fluid ports and/or vacuum ports from being covered and/or blocked by tissue structures (e.g., the tongue and/or cheeks). The sidewall, for example the vertical sidewall 264, has a flexible lip seal (not shown). The lip seal has a divot. A vacuum port is located in or near the divot, for example to drain fluid from the mouthpiece and/or treatment site. The vacuum strength is manually adjustable, not adjustable, or automatically controlled with or without feedback from sensors. In certain embodiments the fluid delivery rate from all sources is related to the vacuum strength. The vacuum level is optionally set so the fluid outlet has a higher flow rate than the flow rate of fluid inflow from all sources. The vacuum level is set low enough not to damage the tissues. In certain embodiments fluid is introduced at the front of the mouthpiece and the vacuum is applied at the back, for example to control fluid flow from the front to the back of the mouthpiece. The fluid outlets 272 are optionally in the bite panel 268. The vacuum ports 267 are optionally at or near the top and bottom of the sidewalls. In certain embodiments solution exits the buccal or lingual side of the mouthpiece, and enters on the opposite side of the mouthpiece 240. The bite panel 268 is optionally covered, for example, with a layer of foam. The mouthpiece 240 optionally has one or more power conduits 271. The power source 246 has a seal 270 around each power conduit entrance, for example an o-ring, to prevent fluid from coming in contact with the electrical conduit when in use.

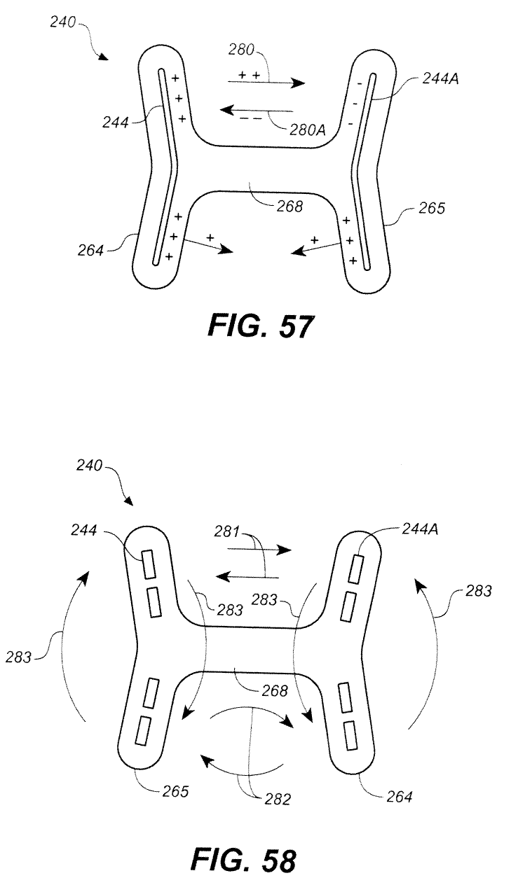

FIG. 57 illustrates that the mouthpiece 240 optionally includes one or more transducers 244 and 244A that create an electric field, such that any charged particles 280 and 280A will experience a force. The transducers 244 include positively and/or negatively charged electrodes 244 and 244A respectively.

Charged particles 280 and 280A that are mobile experience motion as a result of the force created by the electric field.

Power to create the electric field comes from a power source (not shown), for example an electrical cell (i.e., battery) or a connector to an external electrical supply (e.g., an electrical cord or wire and plug). The field is static (an electrostatic field), the field is variable (e.g., alternating, cyclical), or the overall field is created by a combination of electrostatic and variable electric fields. The characteristics of the electric field are optionally adjusted and/or controlled manually and/or through an automated control system. Feedback from sensors is optionally used to automatically adjust the characteristics (e.g., polarity, intensity, rate of change, duration, pulse rate) of the electric field, and/or to alert the user of recommended action.

The sensitizer solution and/or naturally occurring fluids at the treatment site contain particles that experience a force in an electric field (i.e., charged particles). Charged particles in the sensitizer solution are mobile (i.e., free to move under the force of the electric field). In certain embodiments the transducers are positioned, and the characteristics and timing of the electric fields are controlled to force any charged particles in the area of the electric field to become oriented (e.g., line up, align) and/or move in a particular direction and/or pattern, for example to circulate and/or to oscillate toward or away from the treatment site. The motion and/or orientation of the charged particles, due to the presence of an electric field, directly results in the motion and/or orientation of non-charged particles in the solution and/or other fluids at the treatment site due to the properties of the fluids (e.g., viscosity). The flow of solutions in the treatment site is optionally directed, and/or encouraged, and/or inhibited and/or otherwise controlled by features of the cleaning system (e.g., holes, channels, notches, grooves, protrusions) that allow or inhibit solution motion.

In certain embodiments the electric field generating device is separate from the solution delivery system and is used in combination with the solution delivery system and/or the sensitizer solution. The separate electric field generating device is, for example, in the form of a handpiece, and/or a stationary device used to apply an electric field externally, and/or a catheter designed to access and apply an electric field to an internal body surface and/or hollow body organ.

For viscous and/or thixotropic sensitizer solutions, (e.g., gels, foams, sols, suspensions, dispersions, pastes, etc.) movement of solution components as a result of the applied electric field greatly increases the penetration of the sensitizer solution, or some of its compounds (e.g., those that respond most strongly to the electric field), into small spaces (e.g., between teeth, between the teeth and gums, into the alveoli of the lungs, into rough and/or highly folded surfaces (e.g., tongue, stomach wall, intestine wall), into biofilms, into and/or through pores (e.g., pores in bacterial or other organism cell walls and/or membranes, pores in the skin or mucosal surfaces), into porous surfaces (e.g., tooth enamel, tooth dentin, finger and/or toe nails), under toe nails, into the base of hair follicles, into atherosclerotic materials (e.g., arterial plaque), and through cell layers and/or linings and/or membranes (e.g., endothelium, mesothelium, basil lamina, skin). The motion of solution components (e.g., sensitizer ions, compounds that are consumed in chemical reactions, oxygen, catalysts), as a result of the electric field results in higher concentrations of these compounds being located near and/or within the target sites and/or organisms.

The electrodes 244 and 244A are optionally constructed from differing materials in the galvanic series, thereby creating a battery and resultant electric potential difference when exposed to the sensitizer solution and/or fluids in the treatment site, which serve as the electrolyte for the resulting galvanic cell.

The overall efficiency of the sensitizer solution at producing RCS, (e.g., singlet oxygen and other ROS), termed quantum yield, is dependant on the efficiency with which the sensitizer absorbs energy (e.g., photons, ultrasonic energy) and then transfers this energy to compounds such as oxygen. The orientation of the sensitizer molecules to the energy source affects the likelihood that incoming energy will be absorbed and therefore affects the quantum yield. In certain embodiments the orientation, magnitude, and timing of the electric field is controlled to force charged sensitizer particles to orient themselves so as to increase their likelihood of absorbing incoming energy, and therefore increase their quantum yield of RCS.

FIG. 58 illustrates that the transducers 244 and 244A control the positioning, geometric orientation, flow pattern, characteristics, and activation timing of the sensitizer solution and/or components (e.g., fluids, charged and/paramagnetic particles) of the sensitizer solution.

A first pattern of flow reciprocates (e.g., tidal). The first pattern of flow reciprocates, as shown by arrows 281, between the buccal sidewall 264 and the lingual sidewall 265. The first pattern of flow 281 reciprocates between the bottom and top of the applicator 240. A second pattern of flow, as shown by arrows 282, is horizontal recirculating (e.g., circular or oval) flow. A third pattern of flow 283 is a vertical recirculating (e.g., circular or oval) flow.

The mouthpiece optionally has (not shown) holes, regions of permeable materials, channels, notches, grooves, or combinations thereof, for example to enhance and/or direct flow. The transducers cause any or all of the patterns of flow individually or in any combination. The flow includes charged, magnetic and neutral particles.

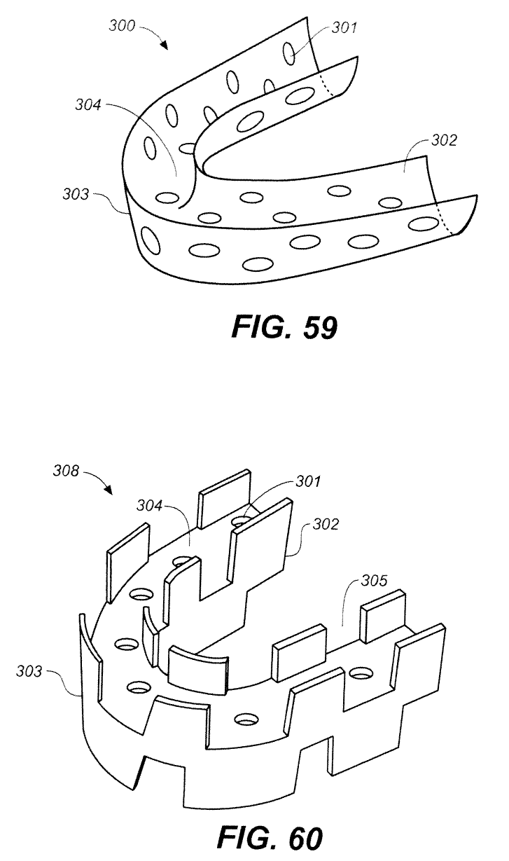

FIGS. 59 and 60 illustrate that the mouthpiece 300 has holes 301. The holes 301 are in the lingual sidewall 302, in the buccal sidewall 303, in the bite panel 304, or in combinations thereof. The holes 301 are from about 0.01 inches in diameter to about 0.2 inches in diameter, for example about 0.01 inches, or about 0.04 inches, or about 0.08 inches, or about 0.12 inches, or about 0.16 inches, or about 0.2 inches in diameter. In certain embodiments the mouthpiece 300 is constructed wholly or in part from one or more porous materials.

FIG. 60 illustrates that the mouthpiece 308 has a notch 305 in the buccal sidewall 303, in the lingual sidewall 302, or in combinations thereof. The bite panel 304 has holes 301 in combination with the notches 305 in the sidewalls 302 and/or 303. The mouthpiece 308 is optionally constructed wholly or in part from one or more porous materials.

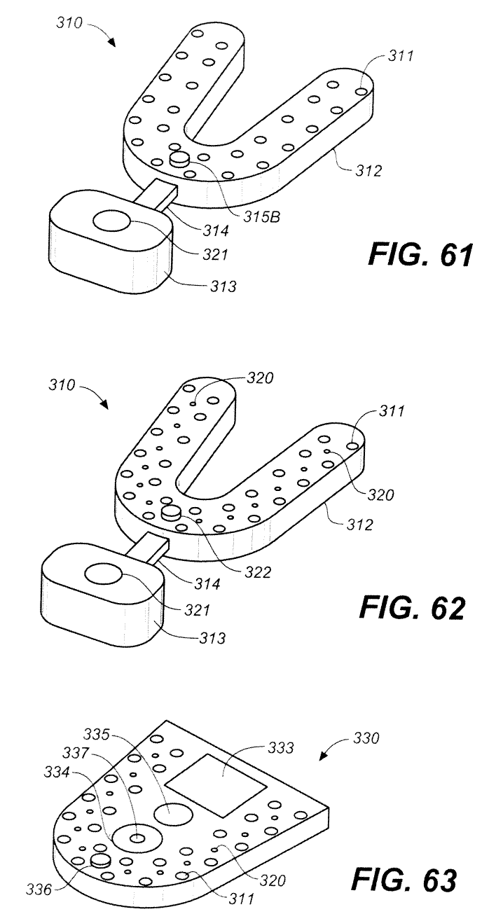

FIG. 61 illustrates that the mouthpiece 310 has light sources 311. The light sources 311 are in and/or on the bite panel 312. The mouthpiece 310 has a flat configuration. The mouthpiece 310 is absent of sidewalls 302 and/or 303 (as shown previously). The mouthpiece 310 is in electrical communication with a power source 313. A power connector 314 can attach the mouthpiece 310 to the power source 313. The power source 313 is optionally one or more electrical cells (e.g., batteries) and/or a connection to an external power supply (e.g., an electrical wall outlet, an infinite bus supply). The power source 313 optionally contains the illumination device (not shown). The power connector 314 and/or the power source 313 are removably attached to the bite panel 312. The power connector 314 is removably attached to the power source 313.

The power source 313 is configured to activate the light sources 311 to emit light energy. The power source 313 delivers power to the illumination device. The illumination device delivers energy, for example through optical fibers and/or wires, to the light sources 311. Each light source 311 has or is each illumination device. For example, each light source is optionally an LED. In certain embodiments the mouthpiece 310 has first and second light controls 321 and 315B. The power source 313 has the first light control 321. The bite panel has the second light control 315B. The second light control is configured to be activated by a user's lips, teeth and/or tongue.

FIG. 62 illustrates that the mouthpiece 310 has one or more fluid outlets 320 and/or light sources 311. The mouthpiece 310 has internal fluid passages in communication with the fluid outlets. The mouthpiece optionally has external channels (not shown) that aid in the distribution of the sensitizer solution over the treatment site. The fluid outlets 320 are in a staggered configuration with the light sources 311 along the mouthpiece 310. A power connector 314 attaches the mouthpiece 310 to the power source 313. The internal channels and fluid outlets 320 of the mouthpiece 310 are in fluid communication with the delivery conduit (not shown), for example through a connector (not shown) on the power source 313 and/or the power connector 314. The mouthpiece 310 is integral with, or fixedly or releasably attached to the delivery conduit. The mouthpiece is connected, through for example a delivery conduit (not shown), to one or more external fluid sources, for example a fluid cartridge and/or SDS (not shown). The mouthpiece is optionally connected directly to one or more external fluid sources, for example in the form of a syringe.

The mouthpiece 310 has the fluid control 321. The fluid control 321 is on the power source 313 (as shown) and/or the power connector 314 and/or the bite panel 312. The power source optionally contains a closed reservoir (not shown) of sensitizer solution, a pump (e.g., piezoelectric pump) and fluid controllers (e.g., valves, flow channels). The pump and optionally the fluid controllers are in fluid communication with the reservoir and with the fluid outlets.

The reservoir 334 in the mouthpiece 330 in FIG. 63 is in fluid communication with a seal 337 that has an aperture (not shown). The seal 337 is self-sealing. The aperture is self-sealing. The aperture is configured to receive a connector and/or a needle (not shown). The connector and/or needle are connected to a supply of sensitizer solution, for example a pressurized cartridge or syringe (not shown), which is used to fill the reservoir 334. The pressurized cartridge is optionally sized to enable multiple fillings of the mouthpiece reservoir 334. The reservoir 334 is filled with sensitizer solution via an injection through the aperture and/or the seal. The reservoir has a reservoir window (not shown). The reservoir window displays how much fluid is in the reservoir. The reservoir is in fluid communication with the fluid outlets 320.

FIG. 63 illustrates that the mouthpiece has a bite panel configured as a plane surface that is substantially or completely covered by light sources 311 and/or fluid outlets 320. The mouthpiece 330 has one or more power sources 335. The mouthpiece 330 is configured to fit across the mouth, including over the tongue, and/or across the hard and/or soft palate. The bite panel has the light control 336 and/or fluid control (not shown) and/or other control (not shown).

The mouthpiece 330 has a closed reservoir 334. The reservoir is in fluid communication with a seal 337 that has an aperture (not shown). The seal 337 is self-sealing. The aperture is self-sealing. The aperture is optionally configured to receive a connector and/or a needle. The connector and/or needle are connected to a supply of sensitizer solution, for example a pressurized cartridge. The pressurized cartridge is optionally sized to enable multiple fillings of the mouthpiece reservoir. In certain embodiments the reservoir 334 is filled with sensitizer solution via an injection through the aperture and/or the seal 337. The reservoir has a reservoir window (not shown). The reservoir window displays how much fluid is in the reservoir. The reservoir is in fluid communication with the fluid outlets 320. The fluid outlets 320 are configured to elute (e.g., slowly release) the sensitizer solution for example through a porous matrix (not shown) integral with and/or attached to the mouthpiece 330. The light control 336 optionally controls the release of the sensitizer solution.

The element shown as the light control 336 in FIG. 63 is instead or additionally a transducer 333 (e.g., light source, and/or an ultrasonic transducer, etc.).

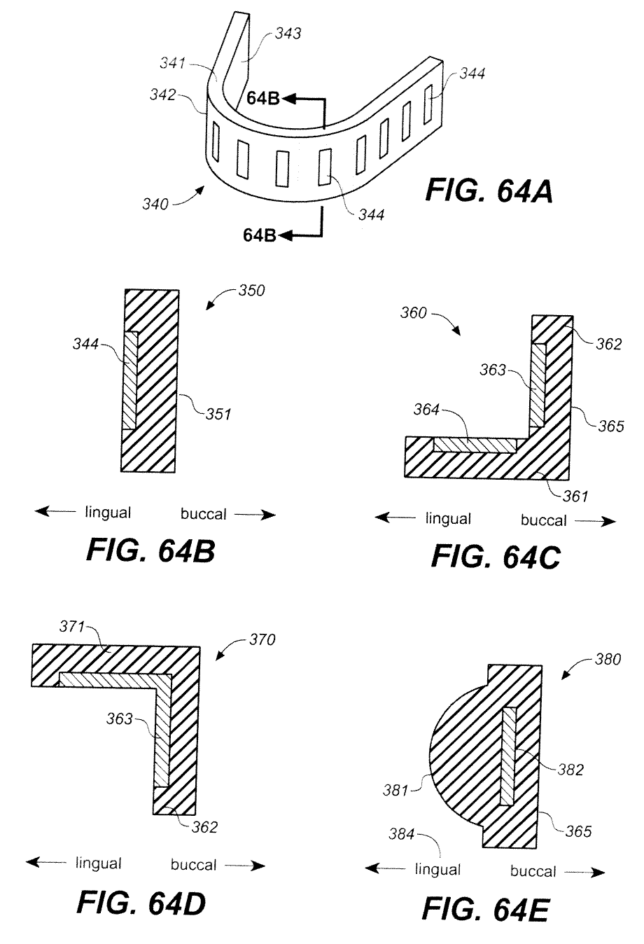

FIGS. 64A and 64E illustrate that in certain embodiments the mouthpiece 340 is configured as a sidewall 341 buccal 342 or lingual 343 with or without (as shown) a bite panel. In certain embodiments the mouthpiece 340 is transparent to the frequency of energy emitted by the light source 344 and/or within a range highly sensitive to the sensitizer solution (not shown). The light sources 344 are optionally on the buccal 342 or lingual 343 side of the mouthpiece 340. The mouthpiece 340 optionally has no bite panel.

FIG. 64B illustrates that the light source is on the lingual side of the mouthpiece 350. The mouthpiece 350 optionally has no bite panel. The mouthpiece 350 has a reflector 351. The side of the reflector 351 closer to the light source 344 is reflective to the frequency of energy emitted by the light source 344 and/or within a range highly sensitive to the sensitizer solution. The reflector 351 is optionally buccal to the light source 344. The reflector 351 is optionally on the opposite side of the light source 344 to the treatment site side.

FIGS. 64C and 64D illustrate a variation of mouthpiece 360 that has a bite panel 361 and a buccal sidewall 362. The buccal sidewall 362 has a first light source 363. The bite panel has a second light source 364. The buccal sidewall 362 and the bite panel 361 have the reflector 365. The reflector 365 is below the second light source 364 in the bite panel 361. FIG. 64D illustrates that the mouthpiece optionally has a single light source 363 that is in the buccal sidewall 362 and/or the bite panel 371.

FIG. 64E illustrates another variation of the mouthpiece 380 that has one or more diffusers 381. The diffusers 381 are geometric configurations designed to diffuse the energy emitted by the light source 382. The diffuser 381 optionally has a semi-circular or otherwise convex cross-section. The diffuser 381 is optionally aligned with the light source 382. The diffuser is on the lingual side 381 of the light source 382. The diffuser 381 is on the treatment site side of the light source 382.

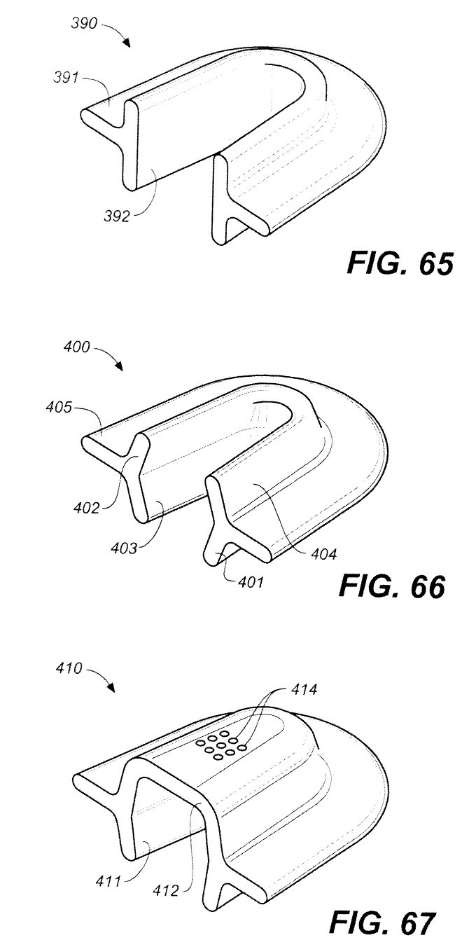

FIG. 65 illustrates that the mouthpiece 390 has a bite panel 391 and a single sidewall, lingual sidewall 392 shown. The bite panel 391 meets the sidewall 392 at a complete or substantial right angle. Optionally the mouthpieces shown in FIGS. 65 through 68 have light outlets and/or fluid outlets as shown herein.

FIG. 66 illustrates that the sidewall, lingual sidewall 403 shown, has a sidewall bottom 401 and/or a sidewall top 402. The buccal sidewall 403 optionally has the same characteristics, but is not shown for illustrative purposes, although the mouthpiece 400 has a bite panel 405 and/or the buccal sidewall 403 and/or the lingual sidewall 404 or no sidewalls. The sidewall bottom 401 forms a substantially non-zero angle with the sidewall top 402. For these embodiments the lingual sidewall 403 may be replaced by a buccal sidewall (not shown).

FIG. 67 illustrates that mouthpiece 410 and the lingual sidewall 411 are attached to a palate panel 412. The palate panel 412 has light sources and/or fluid outlets 414. In certain embodiments the mouthpiece and the transducers provide therapeutic coverage for all soft and hard tissues of the oral cavity (e.g., tongue, soft and/or hard palate, teeth, gums, cheeks, lips).

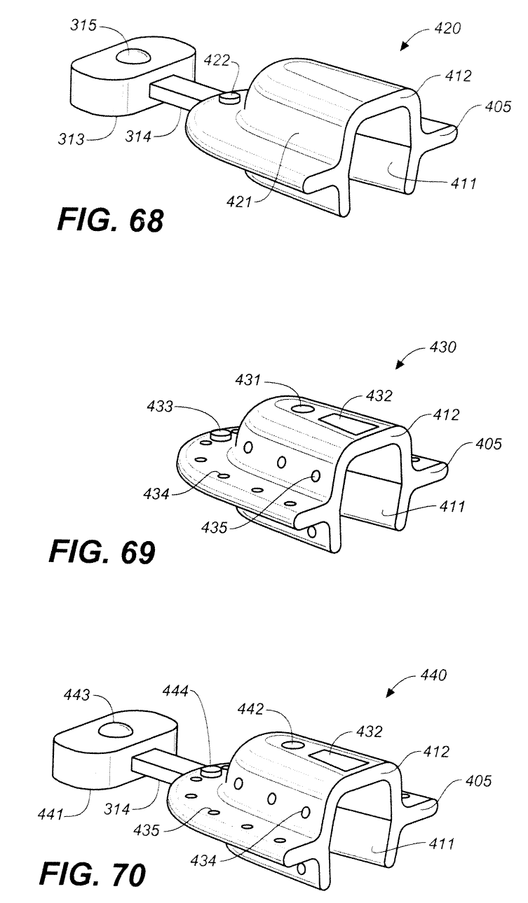

FIG. 68 illustrates that substantially the entire mouthpiece 420 (as shown), and/or substantially the entire lingual and/or buccal sidewall, and/or substantially the entire palate panel 412 are the light source 421. Substantially the entire mouthpiece 420 (as shown), and/or substantially the entire lingual and/or buccal sidewall, and/or substantially the entire palate panel 412 are made from a transparent or translucent material to the frequency of the energy emitted. In certain embodiments the illumination device and/or other originator of the energy are in the power source 313, and/or power connector 314, and/or the remainder of the mouthpiece 420. The power connector 314 and/or power source 313 are removably attached to the bite panel 405 and/or sidewalls 411 and/or palate panel 412. The power connector 314 is optionally removably attached to the power source 313. The mouthpiece 420 is optionally made from a material transparent and/or translucent to the frequency of the light energy.

In certain embodiments one or more portions of the mouthpiece 420 are made from and/or coated with a material (not shown) that does not allow the transmission of energy from one and/or more of the transducers to penetrate and/or pass through it. For example, regions of the mouthpiece are colored and/or reflective and or coated with a colored and/or reflective material that does not allow light from the light sources to penetrate and/or pass through it. This allows for activation of the sensitizer solution to be limited to certain areas (i.e., treatment sites).

FIG. 69 illustrates that the mouthpiece 430 optionally has a power source 431 and/or one or more transducers 432 in the palate panel 412. In certain embodiments the palate panel 412 is extended rearward (not shown) past the ends of the lingual sidewalls and/or bite panel 405 to cover more of the tongue and/or palate surfaces. The space between the lingual sidewalls 411 of the mouthpiece 430 is sized and shaped to comfortably receive the tongue. The lingual sidewall 411 optionally has one or more transducers with their energy emissions oriented toward the tongue, for example the sides of the tongue, or toward the teeth and gum surfaces. The bite panel 405 optionally has one or more transducers 435. The palate panel transducers are oriented to direct their energy emissions toward the tongue and/or palate (e.g., hard and soft).

In certain embodiments the bite panel, lingual sidewall, and palate panel transducers provide energy sufficient to activate the sensitizer solution over the entire upper and side surfaces of the tongue, surfaces of the teeth and gums, inner surfaces of the cheeks and/or lips, and over the entire hard and soft palate. The bite panel, lingual sidewalls and palate panel optionally contain light sources, ultrasonic energy sources, thermal sources, electric field sources, magnetic field sources, or combinations thereof. The ultrasonic energy distributes the sensitizer solution. The ultrasonic energy can penetrate the sensitizer solution into the textured surface of the tongue. The ultrasound energy can activate any sonosensitizing agent in the sensitizer solution. In certain embodiments the ultrasonic transducers also increase the level of fluid flow and mixing within the oral cavity, for example between the mouthpiece and the oral cavity structures, in the subgingival spaces, around and between the teeth, and deep into the textured surface of the tongue.

In certain embodiments the surface of the mouthpiece, in whole or in part, has features and/or materials that aid in the removal of biofilm (plaque) and/or in the distribution and/or activation of sensitizer solution. The surface of the mouthpiece optionally has soft polymer bristles, for example similar to those found on a toothbrush, and/or closed or open loop material, and/or polymer foam (e.g., open cell, closed cell), and/or a non-soluble gel. The surface features and/or materials are optionally transparent and/or transmissive and/or conductive to the energy emissions of the transducers, for example the polymer bristles can transmit light energy and mechanical energy. By further example, the surface has conductive polymer foam (not shown) that acts as an electrode. The light control 433 (as shown), and/or the fluid outlet 434; and/or the other control is in the bite panel 405. The reservoir is optionally in the palate panel 412, and/or in the bite panel 405, and/or in the lingual and/or buccal (not shown) sidewalls 411.

FIG. 70 illustrates that the mouthpiece 440 has a first power source 441 and a second power source 442. The mouthpiece 440 has a first light control 443 and a second light control 444. In certain embodiments the first light control 443 is a manually actuated pump (e.g., squeeze bulb), for example for delivering sensitizer solution from the reservoir (not shown). The first power source 441 is attached to the palate panel 412, and/or the bite panel 405 (as shown), and/or the lingual and/or buccal (not shown) sidewalls 411, for example, through a power connector 314. The first power source 441 is outside of the user's mouth during use. The second power source 442 is attached to or integral with the palate panel 412 (as shown), and/or the bite panel 405, and/or the lingual sidewall 411 and/or buccal (not shown) sidewalls. The second power source 442 is inside the user's mouth during use.

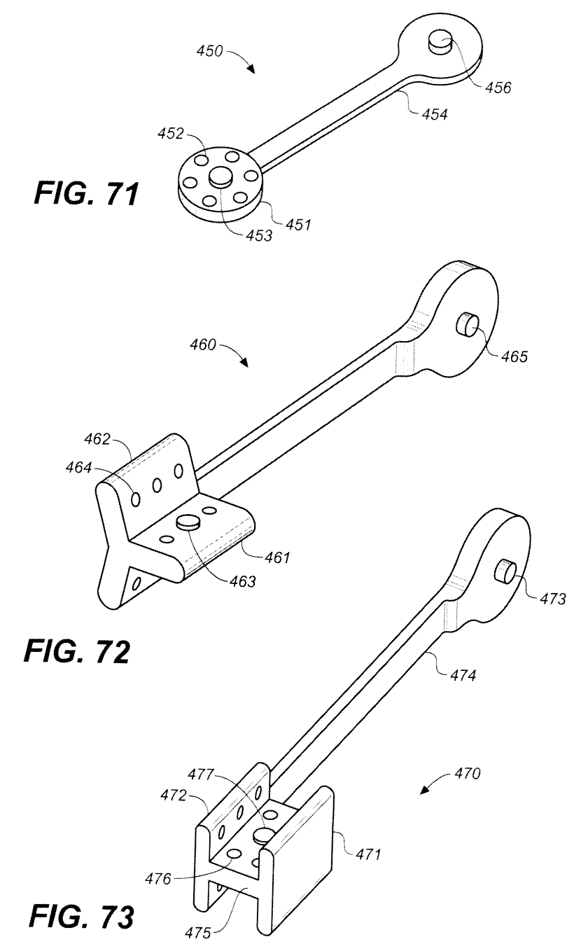

FIG. 71 illustrates that the mouthpiece 450 is optionally a bite block that has a bite panel 451. The bite panel 451 has a shape. The shape is regular (e.g., circle, polygon), or irregular, or customized to reflect the geometry of the individual users anatomy. The bite block 450 has light sources 452 and/or separate fluid outlets (not shown). The bite block 450 is optionally in electrical communication with a power source, for example, located in a handle 454. The handle 454 extends from the bite panel 451. The handle 454 optionally has a first light control 456. The bite panel optionally has a second light control 453. This second light control is optionally an interlock.

The second light control 453 is configured to be activated by the user's lips, and/or teeth and/or tongue. The bite block 450 is in fluid communication with the delivery conduit. The bite block 450 elutes the sensitizer solution, for example through a matrix integral with and/or attached to the bite block 450. The illumination device is placed in the handle 454. The illumination device is in energy communication with the light sources, for example via an optical fiber or conductive wire.

FIG. 72 illustrates that the bite block 460 has a bite panel 461 and a sidewall 462. The bite panel 461 optionally meets the sidewall 462 at a substantial or complete right angle. The sidewalls optionally meet the bite panel at an angle that is not a right angle, for example to improve the comfort of the device in the mouth of the user or to make the sidewall surface be more parallel to the treatment site surface, for example the inner and/or outer surface of the teeth. In certain embodiments the bite blocks have a longitudinal axis that is straight or substantially straight (as shown). In certain embodiments the bite blocks longitudinal axis is curved (not shown), for example to better match the curvature found in the anterior teeth. First light control 465 and second light control 463 are shown which control light source 464.

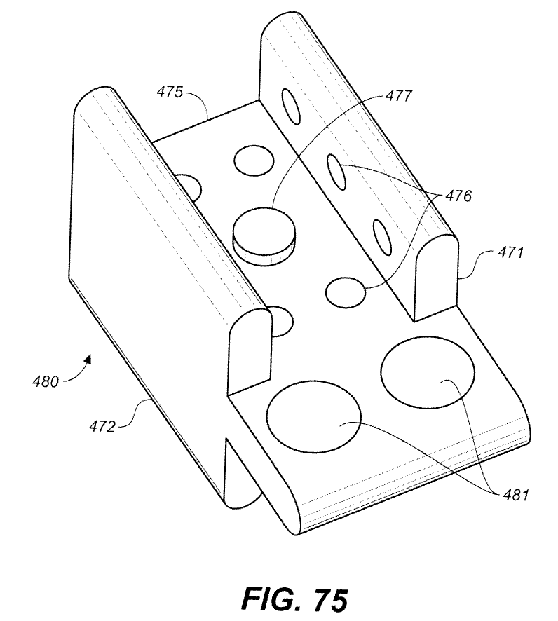

FIGS. 73 and 75 (without handle 474) illustrate that the bite block 470 or 480 is connected or is separate from the handle 474 and has a lingual sidewall 471 and a buccal sidewall 472. In certain embodiments the lingual and buccal sidewalls extend to only one side of the bite panel to give a geometry with a single channel (not shown).

In certain embodiments the transducer is configured to emit energy, for example every 30 minutes for about 30 seconds to about 5 minutes, and/or every hour for about 30 seconds to about 5 minutes.

The bite block 470 and/or 480 are made in whole or part from a material transparent and/or translucent to the frequency of the energy emitted.

The bite block 470 and/or 480 and/or a mouthpiece are made from, in whole or in part, or partially or wholly coated with an absorbable material, for example a polymer matrix. In certain embodiments the bite block 480 and/or a mouthpiece is soaked or otherwise filled with the photosensitizer solution (not shown). The bite block 470 and/or 480 and/or a mouthpiece elutes photosensitizer solution during use. The mouthpiece and/or bite block 470 and/or 480 are optionally made from materials that withstand repeated dishwasher washing, and are sealed such that sensitive components (e.g., a controller) are safe during repeated dishwasher washing.

In certain embodiments the mouthpiece and/or bite block 480 fit to the shape of the patient's teeth and/or gingiva and/or tongue and/or palate and/or oral cavity. In certain embodiments the mouthpiece and/or bite block 480 are configured to produce orthodontic therapy, for example, as described by Chisti et al. in U.S. Pat. Nos. 6,210,162 and 6,227,851, and by Phan et al. in U.S. Pat. No. 6,299,440.

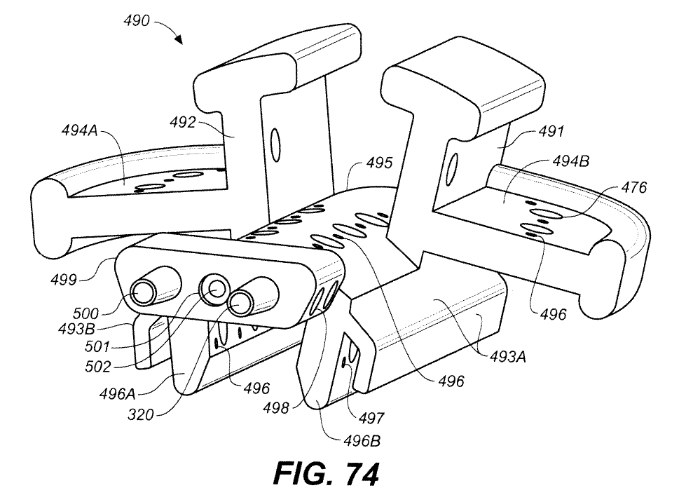

FIG. 74 illustrates that a bite block 490 is configured to apply force to structures of the oral cavity. The force, for example a retraction force, is used to retract tissue, for example from a treatment site. Friction from the compression of some elastomeric or other compressive material (not shown) on the inside of the bite block 490 attaches the bite block 490 to tissue. The compressive material is optionally transparent, translucent, and/or conductive to energy emitted by the transducer. The compressive material is optionally away from the treatment site. The user optionally bites on the bite block 490. The length between buccal and lingual sidewalls is smaller than the width of the teeth. (Lingual and buccal are interchangeable depending on the placement in the mouth). The bite block 490 has a high friction material on the inside of the sidewalls (not shown). The sidewalls 496A and 496B are rounded and/or tapered, for example to allow the device to be placed easily, for example without catching on the edges of the occlusal surfaces of the teeth. The sidewalls 491 and 492 above the bite panel 495 are squeezed together to increase the gap between the lower sidewalls 496A and 496B so that the teeth can fit between the sidewalls (e.g., clothes pin style). In another embodiment the buccal sidewall 491 and lingual sidewall 492 are hinged at the bite panel with spring elements trying to squeeze the bottom sidewalls 496A and 496B together. The upper sidewalls 491 and 492 have a flair to attach to a deployment tool (not shown). A texture or high friction material (not shown) are on the outside of the bite block 490. In certain embodiments the mouthpiece has one or more retraction sidewalls 494A, 494B, 493A and 493B, for example to hold tissue out of the treatment and/or operational site. The retraction sidewalls 494A and/or 494B have a flair. The flair is sized and positioned to contact tissue near the treatment site. The contact area between the flair and the tissue is larger than the contact area if the retraction sidewalls contacted the tissue directly. The retraction sidewalls 494A and/or 494B can be positioned adjacent to the treatment site. Other features include fluid inlet 320, fluid outlet 500, seal 501, power conduit 502, fluid ports 496, vacuum port 497, light source 498, power source 499, and light source 476.

The bite block 490 is optionally used during invasive oral surgery. The sidewalls and/or bite panel are optionally planar and/or curved, for example to allow them to better fit the anatomy, (e.g., the curved shape of the front teeth). The sidewalls 494A and 494B are optionally adjustable, for example to control how far the retraction sidewall extends away from the buccal 491 or lingual 492 sidewall. In certain embodiments the retraction sidewall is attached to the buccal 491 or lingual 492 sidewall through a hinged joint (not shown). The hinged joint optionally has a ratchet and pawl mechanism, high friction at the hinge, and/or a releasable hinge lock. Any of the bite block surfaces optionally has one or more transducers and/or fluid ports and/or vacuum ports. The transducers optionally emit white light.

FIGS. 76 and 77 illustrate an applicator 510 wherein the head 511 is square or rectangular. The head 511 is fixedly or releasably attached to or integral with the neck 512. The neck 512 is curved, deformable, flexible, or combinations thereof. All the controls 513A, 513B, 513C on the neck 512. The handle 514 optionally is a pressurized container. The head 511 is fixedly or releasably attached or integral with the pad 515. The pad 515 is a sponge. The pad includes pad holes. The pad holes are configured to align with the fluid outlets and/or transducers. The pad has no holes. The control 513C is configured to release the head from the neck or the neck from the handle. The neck 510 is snap fitted or screwed onto the handle.

The head 511 includes a valve, for example to control the release of the sensitizer solution.

The applicator is in one aspect in the form of a mop. The pad is on a separate device, for example a mop, and the remainder of the applicator is mounted onto the handle of the mop. Controls for the release of solution and/or activation illumination are near the top of the mop handle. The pad completely surrounds the applicator. The sponge applicator continuously emits the activation illumination and release sensitizer solution, for example when squeezing the sides of the sponge, or pushing down on the sponge.

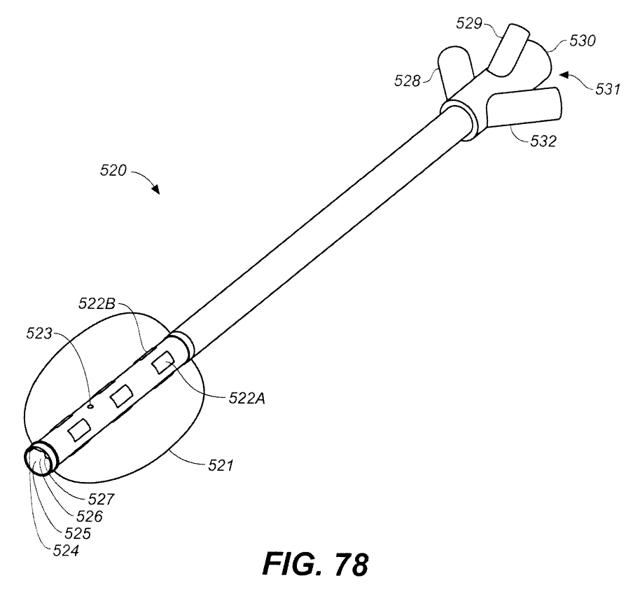

FIGS. 78, 79, and 80 illustrate that the applicator also takes the form of a catheter 520. The catheter 520 has one or more balloons. The catheter 520 has one or more transducers. The transducer is fixedly attached to and/or integral with the catheter. The catheter has polymeric layers. The transducer is laminated between the polymeric layers.

The catheter 520 has one or more lumens. The lumen is configured to transport fluids and/or gases along the length or a portion of the length of the catheter. The lumen contains one or more conductors (e.g., electrical, optical). One end, for example the distal end, or both ends of the lumen is closed, for example by filling the end with adhesive.

A break (e.g., hole, cut, skive) is created anywhere along the length of a lumen to create a connection between the exterior of the catheter and the interior of a lumen.

The catheter has a central lumen. The central lumen is configured to accept different devices (e.g., guide wire, introducer, second catheter) and/or to allow for the delivery of fluids, for example blood, distal to the distal balloon.

The cleaning system has a first and a second catheter. The second catheter is slidably attached within the central lumen of the first catheter.

The catheter is used concomitant with other catheter-based systems. Other catheter-based systems are diagnostic and/or therapeutic ultrasound, angioplasty, stents and/or stent delivery, thrombus removal, or combinations thereof.

The sensitizer solution is delivered into the circulatory or other systemic fluid system (e.g., the lymphatic system) independently of a catheter (e.g., injection, I.V. fluid, catheterization). The catheter is without a fluid source.

The catheter 520 has one or more connectors. The connectors are fixedly or removably attached to the catheter. The connector has a valve. The connector is in fluid and/or electrical communication with one or more lumens and/or one or more transducers and/or one or more energy sources (e.g., electrical, light, ultrasound) and/or one or more fluid and/or vacuum sources (e.g., SDS, wall suction, infusion pump, syringe, power injector, intra-venous bag).

The catheter 520 is configured to deliver the solution (e.g., sensitizer solution, saline, lactated ringers, contrast agent, drugs) and/or energy required to distribute and/or activate the sensitizer solution to a localized region, such as a hollow body organ, for example as disclosed in U.S. Pat. No. 6,159,236 by Biel, U.S. Pat. Nos. 6,176,842 and 6,527,759 by Tachibana et al., U.S. Pat. No. 6,290,689 by Delaney et al., U.S. Pat. No. 6,425,877 by Edwards, U.S. Pat. No. 6,527,979 by Constanz et al., U.S. Pat. No. 6,733,474 by Kusleika, U.S. Pat. No. 5,876,374 by Alba et al., which are all incorporated by reference herein in their entirety.

The sensitizer solution is delivered through the catheter 520 to a treatment site, for example a hollow body organ (e.g., blood vessel, stomach, esophagus, trachea, intestine). The solution is confined to the treatment site for example by the use of sealing structures, for example balloons. The balloon is constructed from a compliant material, for example an elastomer. The balloon is constructed from a flexible (i.e., compliant), or semi-compliant material, for example an elastomeric (e.g., silicone, silicone RTV, latex, vulcanized rubber, buna rubber, Viton®, neoprene, fluorosilicone rubber, EPDM rubber, nitrile rubber, polyurethane, Santoprene®), and/or polymeric (e.g., polyethylene (LDPE, LLDPE, HDPE), polypropylene, polyvinylchloride (PVC), polystyrene, nylon, polyester, mylar), and/or metal foil, and/or metallized polymeric and/or elastomeric material. The balloon is constructed, in whole or in part, from a microporous material. The balloon, in whole or in part, has pores that pass through the balloon material and connect the exterior of the balloon to the interior of the balloon. The pores range in size from about 0.1 microns to about 2 microns. The balloon has no pores. The sensitizer solution is delivered to the target site through the pores in the balloon. The surface and/or volume of the balloon material is coated and/or impregnated and/or saturated with compounds (e.g., sensitizer solution, sensitizer, production increasing compounds, (such as catalysts), peptides, activation compounds). The balloon is configured to deliver energy to the treatment site. The balloon is configured to be an electrode. The catheter is used for a therapeutic vascular treatment and/or diagnosis (e.g., angioplasty, coronary stent placement). The catheter elements and solutions used in conjunction with the catheter are biocompatible and/or sterile.

FIG. 78 illustrates that the catheter has a balloon 521. The balloon is located at the distal end of the catheter 520. The catheter includes one or more transducers 522A and 522B located in the region of the balloon, for example between the locations where the balloon is attached to or integrated with the catheter.

The balloon and/or transducers are deployed to the adjacent site. The balloon is expanded, for example by filling the balloon with the sensitizer solution. The sensitizer solution moves through pores in the balloon and contact the treatment site. The sensitizer solution is delivered at 523 to the balloon, for example, expanding or maintaining expansion of the balloon. The balloon is expanded from about 10 seconds to about 30 minutes. Energy from the transducers is applied before and/or during and/or after the balloon is expanded and/or re-expanded. The balloon is contracted (e.g., deflated), repositioned and re-expanded (e.g., re-inflated) in the same or a new location.

The balloon is covered by a sheath (not shown), for example, as disclosed in U.S. Pat. No. 5,876,374 by Alba et al., and U.S. Pat. No. 6,733,474 by Kusleika. A closed intra-sheath volume is formed between the exterior surface of the balloon and the interior surface of the sheath. The sheath is fixedly or removably or slidably attached to the catheter. The sheath is constructed from a flexible (i.e., compliant), or semi-compliant material, for example an elastomeric (e.g., silicone, silicone RTV, latex, vulcanized rubber, buna rubber, Viton®, neoprene, fluorosilicone rubber. EPDM rubber, nitrile rubber, polyurethane, Santoprene®), and/or polymeric (e.g., polyethylene (LDPE, LLDPE, HDPE), polypropylene, polyvinylchloride (PVC), polystyrene, nylon, polyester, mylar), and/or metal foil, and/or metallized polymeric and/or elastomeric material.

The sheath is made from a microporous material. The sheath has pores that can pass through the sheath material. The pores connect the exterior of the sheath to the interior of the sheath. The pores range in size from about 0.1 microns to about 2 microns. The sensitizer solution is delivered to the target site through the pores in the sheath. The surface and/or volume of the sheath material is coated and/or impregnated and/or saturated with compounds (e.g., sensitizer solution, sensitizer, production increasing compounds, (such as catalysts), peptides, activation compounds). The sheath is configured to deliver energy to the treatment site. The sheath can be configured to be an electrode.

The sheath extends from the distal end of the catheter to approximately even with the proximal end of the balloon. The sheath is fixedly attached to the proximal sleeve of the balloon, and/or the distal sleeve of the balloon. The sheath extends beyond the proximal end of the balloon, for example by a distance from about 0.1 in to about 2 inches. The sheath is fixedly attached directly to the catheter at a position proximal to the proximal end of the balloon or distal to the distal end of the balloon. The sheath extends from about the distal end of the balloon to near the distal end of the connector, for example the central lumen 530 connector. The distance between the proximal end of the sheath and the distal end of the connector is approximately equal to or greater than the length of the balloon.

The sheath is slidably attached to the catheter. The sheath slides axially along the catheter from a first position to a second position. In the first position the sheath completely or substantially covers the balloon. In the second position the sheath is proximal to the balloon. The balloon has no portion of the expandable portion of the balloon inside the sheath. The proximal end of the sheath contacts the distal end of the connector in the second position of the sheath.

The intra-sheath volume is filled with a solution (e.g., high pH sterile saline (e.g., greater than 9), low pH sterile saline (e.g., lower than 3), drug (such as heparin), sensitizer solution).

The catheter 520 has a non-porous balloon covered by a porous expandable sheath. The intra-sheath volume has (e.g., be filled with) the sensitizer solution. The sheath is deployed adjacent to the treatment site. The balloon is expanded, for example with air, sterile saline, or sterile water. Expansion of the balloon forces sensitizer solution through the pores of the sheath and forces the sensitizer solution and/or the sheath into contact with the adjacent treatment site.

The balloon is expanded to a diameter from about 0% to about 75% larger than the natural diameter of the hollow body in which the balloon is deployed. The balloon is deflated and the catheter removed from the patient.

The catheter 520 is configured with a non-porous balloon covered by a porous expandable sheath. The proximal and distal ends of the porous expandable sheath are fixedly attached to the catheter. The attachments (e.g., proximal and distal) between the porous expandable sheath and the catheter are fluid and/or air tight.

The intra-sheath volume is in fluid communication with a lumen in the catheter and a connector, for example through a skive in the catheter located between the proximal and distal sheath attachments but outside of the region of the catheter covered by the balloon. The sheath is deployed adjacent to the treatment site. Solution, for example, sensitizer solution, is delivered into the intra-sheath volume. The balloon is expanded, for example with air, sterile saline, or sterile water. Expansion of the balloon forces solution from the intra-sheath volume through the pores in the sheath and forces the solution and/or the sheath into contact with the treatment site. The balloon is deflated and the intra-sheath volume refilled with solution, for example sensitizer solution. A refilled catheter is used to treat the same or additional treatment sites. The intra-sheath volume is refilled with a solution that is different from the first solution. The first solution, for example, prepares the treatment site for the activity of the second solution. The first and second solutions are, for example, part A and part B respectively, of a two-part sensitizer solution. The balloon is deflated and the catheter removed from the patient.

The catheter has a non-porous balloon and a non-porous expandable sheath. The porous expandable sheath is configured to slidably move between a first position and a second position. In a first position the sheath completely or nearly completely covers the balloon. In a second position the balloon is completely or nearly completely outside of the sheath.

The balloon, in whole or in part, is coated with a solution, for example in a gel form. The solution substantially or completely fills the intra-sheath volume. The solution is applied to the catheter with the sheath in the first or second position. To prevent or limit the movement of solution along the catheter in a proximal direction, the inner diameter of the sheath just proximal to the intra-sheath volume is substantially equal (e.g., smaller by 0.003 inches or less, larger by 0.010 inches or less) to the outside diameter of the catheter.

The balloon and sheath materials and/or texture, and the solution composition are designed so that the solution adheres more tightly to the balloon than to the sheath. The balloon has a lightly textured surface. The solution has a bioadhesive that adheres to the surface of the balloon. The sheath is made from or lined or coated with a lubricious and/or low friction material (e.g., Teflon).

The solution remains in position when the sheath is moved from a first to a second position. The catheter, with the balloon deflated and the sheath in the first position, is positioned so that the sheath, in the region of the balloon, is adjacent to a treatment site. The sheath is moved from a first position to a second position. In the second position, the balloon surface is adjacent to the treatment site. The balloon is expanded to a diameter from about 0% to about 75% larger than the natural diameter of the hollow body in which the balloon is deployed. The balloon is deflated and the catheter removed. The solution is configured, for example through the incorporation of a bioadhesive to adhere to the tissue at the treatment site. Additional features include power conduit connectors 528 and 532, balloon inflation connector, 529, proximal connector 531, balloon inflation port 523, power conduit lumen, 524 and 527, central lumen 525, and balloon inflation lumen 526.

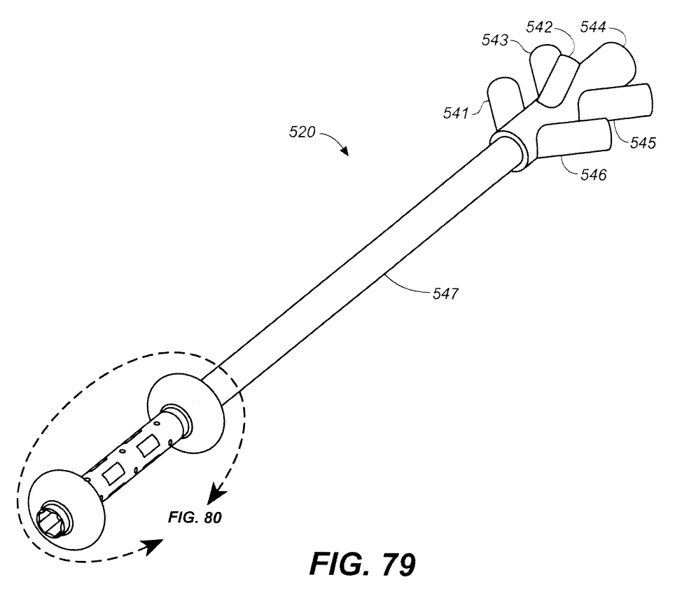

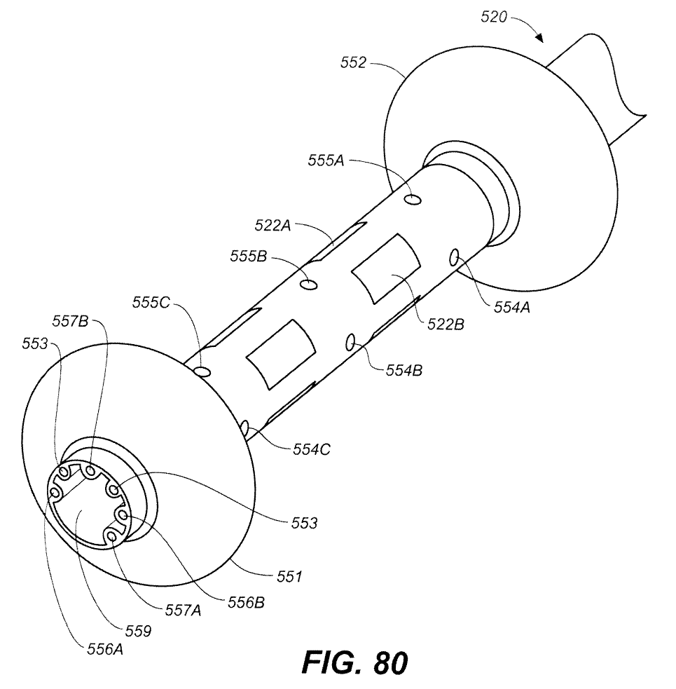

FIGS. 79 and 80 illustrate that the catheter has a first balloon 551 and a second balloon 552. The first balloon is distal to the second balloon by a balloon gap. The balloon gap is equal to a treatment section of the catheter. The treatment section includes one or more transducers 522A and 522B (e.g., light sources, ultrasound sources, electric field sources, magnetic field sources, heat sources, or combinations thereof).

The catheter 520 is inserted into a hollow body organ with the balloons deflated. The treatment section is deployed adjacent to the treatment site. When the catheter reaches the treatment site, the balloons 551 and 552 are inflated. The balloons are each in fluid communication with their own balloon inflation lumen and balloon inflation connector allowing them to be inflated individually. The proximal and distal balloons, for example the first and second balloon, are optionally in fluid communication with a single balloon inflation lumen 553 and balloon inflation connector 541 and 546 allowing them to be inflated simultaneously. The balloons 551 and 552 are inflated until they form a substantially or completely fluid tight seal with the tissue of the hollow body in which the catheter 520 is deployed. The area of the hollow body parallel with the treatment section is the treatment site. The exterior of the treatment section of the catheter is in fluid communication with fluid ports 554A, 554B, and 554C and vacuum ports 555A, 555B, and 555C. The fluid ports 554A-554C are in fluid communication with one or more solution delivery lumen 556A and 556B and one or more solution delivery connectors 545. Vacuum ports 555A-555C are in fluid communication with one or more vacuum lumens 557A and 557B and one or more vacuum connectors 543. Fluid is delivered to the treatment site through the fluid ports. Fluid is removed from the treatment site through the vacuum ports and/or the central lumen 559. Fluid is optionally delivered to the treatment site through the fluid ports at the same time that fluid is being removed from the treatment site through the vacuum ports. Additional features include power conduit connector 542, central lumen connector 544 and catheter shaft 547.

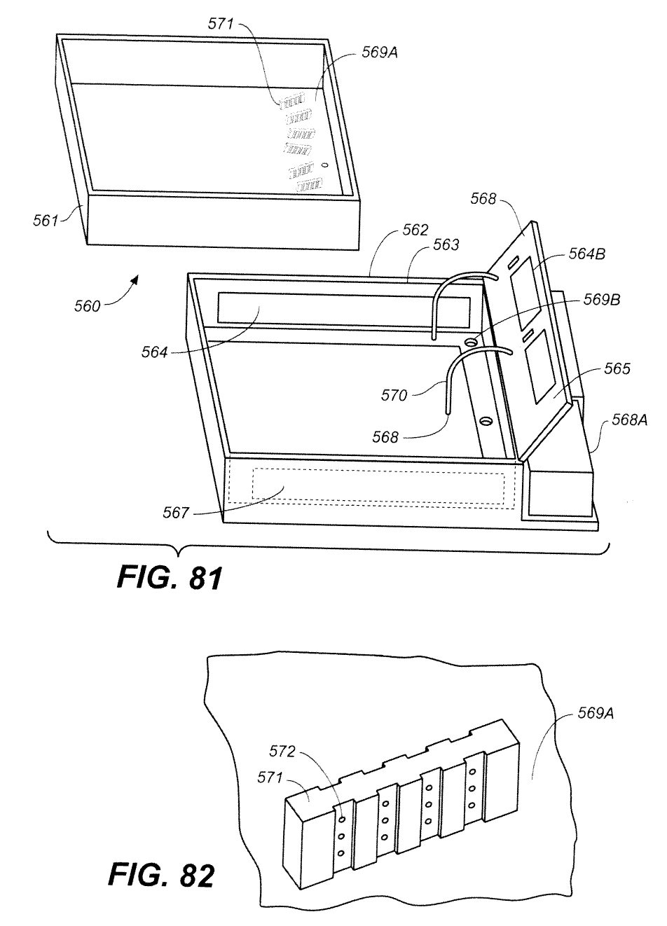

FIG. 81 illustrates that the cleaning system 560 is configured as a bath or soaking device. The applicator 561 is configured as a soaking tray that is removably attached or integral with the remainder of the cleaning system 560. The applicator 561 is transparent or translucent to the wavelength of energy emitted during use.

The applicator 561 is slidably received by an applicator cavity 562. The applicator snaps fit or is held solely by gravity in the applicator cavity 562. The applicator cavity 562 is formed in part or in whole by applicator cavity walls 563. The applicator cavity walls 563 and/or floor have one or more light sources 564 and/or illuminating devices 564B and 564C. The cleaning system 560 has a cleaning system plate 565. The cleaning system plate 565 is rotatably attached to one or more applicator cavity walls 563. The cleaning system plate 565 includes one or more light sources 564B and 564C and/or illuminating devices 564B and 564C. The cleaning system has one or more fluid inlets and/or outlets 568, 569A and 569B.

One or more first applicator fixators 569A on the applicator 561 are configured to attach to one or more second applicator fixators 569B on the applicator cavity 562. The applicator fixators 569A and 569B are configured to attach the applicator 561 to the applicator cavity 562. The applicator fixators 569A and 569B are configured to transmit sensitizer solution and/or power (e.g., electricity) and/or data (e.g., desired energy frequency, sensor signal) between the applicator 561 and the remainder of the cleaning system.

The cleaning system is designed to deliver heat into the sensitizer solution, for example through an electric heating coil. The heating coil is in the applicator, and/or in the base and/or applicator cavity walls, and/or in the SDS.

The delivery conduits 570 are configured to deliver sensitizer solution into the applicator 561 and/or the applicator cavity 562. The cleaning system 560 shown in FIG. 81 has fluid, and/or light and/or other controls (not shown).

The sensitizer is in the form of a solid (e.g., tablet, block, pellet(s), crystal), hereafter sensitizer tablet. The sensitizer tablet is placed in the applicator, and/or in the SDS, and/or in a fluid path so that it is exposed to the fluid in the applicator. A fluid or combination of fluids in which the sensitizer tablet is soluble (e.g., water, or hydrogen peroxide, or isopropyl alcohol, or combinations thereof) is added directly to the applicator and/or delivered from the SDS, for example a fluid reservoir in and/or on the SDS. The SDS circulates the fluid and thereby increases the rate at which the sensitizer tablet dissolves. The cleaning system is designed so that the tablet and/or the fluid in the applicator changes color when a sufficient amount of the sensitizer has dissolved for the cleaning system to be effective.

The applicator 561 has one or more tips 571, for example, protruding from the applicator 561. The tips 571 are in fluid and/or energy communication with the first applicator fixators 569A.

FIG. 82 illustrates that the tip 571 has one or more fluid outlets 572. The tips have light sources also 572. The tips transmit and/or are transparent to the energy emitted from the light source(s) and/or illuminating devices. The tips are positioned along side all or a part of the treatment site, for example the tips are positioned between the fingers and or toes of the user.

The user's body, hands, feet, personal artifacts, clothes, dishware or combinations thereof having the treatment site(s) are placed in the applicator 561. The sensitizer solution is then applied, for example by manually application, and/or by spraying from the delivery conduit(s) and/or the fluid outlets, and/or by soaking, to the treatment site(s). The energy is then emitted from the light source(s) and/or illuminating devices.

The applicator is a mouthpiece. The applicator is a wand. The applicator is a catheter. The applicator is a bath or soaking tray.

The applicator is an aerosol mister. The sensitizer solution is delivered into the respiratory tract.

The transducer is placed in direct contact with the treatment site.

The cleaning system has a pressure regulator, for example to control the pressure at which the solution is released.

The cleaning system 560 is used to alter the color of teeth or dentures, or personal artifacts, or clothing, for example for tooth or denture whitening. The sensitizer solution is in a form that is flowable, for example, an aqueous or non-aqueous solution, suspension, or dispersion such as a liquid or solid aerosol, foam, gel, emulsion (e.g., oil-in-water, water-in-oil), paste, powder, micelle, liquid crystal, liposome, niosomes, sols, sol gel, semisolid or macrosolid suspension or combinations thereof. The sensitizer solution is in a non-flowable form, for example a solid, or crystal. The sensitizer solution is made from a sensitizer mixed with a pharmaceutically acceptable aqueous carrier, for example water such as distilled water, demineralized water, pyrogen-free water, sterile water, or water having combinations of the aforementioned characteristics. “Pharmaceutically acceptable” is acceptable to be included as a component of a composition that comes in contact with a living organism.

Microprocessors are used in any embodiment to control the energy emission profile and its change as a function of time, (e.g., power levels, time activated, fluid flow rates, fluid pulsation pattern, fluid pulsation rate, fluid pulsation duration, energy amplitude, energy intensity, energy frequency, type (e.g., acoustic, thermal, electromagnetic, magnetic field, potential gradient, electric field) of energy emitted, energy pulsation pattern, energy pulsation rate, energy pulsation duration), battery save modes, warnings and other communication with the user, and combinations thereof. Feedback from sensors is used to automatically adjust the transducers energy emission profile, and/or to alert the user of recommended actions, for example through the sounding of a tone and/or the flashing of a light. The cleaning system is configured to allow the user to choose if the energy emission profile is adjusted manually or automatically through, for example, a switch.

One or more solution containers and/or pressurized cartridges are incorporated into existing equipment to provide delivery of the sensitizer solution to a treatment site (e.g., dental equipment (e.g., oral irrigation equipment, rinse equipment, drills, ultrasonic scalers, probes), wound care equipment/devices (e.g., wound irrigation devices), laparoscopic and/or arthroscopic surgical devices (e.g., irrigation devices), liquid ventilators (e.g., ventilators used for total liquid ventilation of the lungs), mechanical gas ventilators (e.g., ventilators used for gas and/or partial liquid ventilation of the lungs), drug delivery devices, for example transdermal delivery devices. The method of liquid ventilation therapy (e.g., total liquid ventilation, partial liquid ventilation (PLV)) is known in the art, for example for the treatment of Acute Respiratory Distress Syndrome (ARDS) and Acute Lung Injury (ALI). The cleaning system is used to treat a patient with liquid ventilation that includes delivering the sensitizer solution to the lung of the patient through total and or partial liquid ventilation of the lung. After a delay, activation energy, for example light, is applied to a target site in the lung. The light has a wavelength that sufficiently penetrates the patient and/or tissues of the target site and activates the administered sensitizer.

### Method of Making

The solutions disclosed herein are manufactured using processes for the manufacture of an aqueous or non-aqueous solution, suspension, or dispersion such as a liquid or solid aerosol, foam, gel, emulsion (e.g., oil-in-water, water-in-oil), paste, powder, solid, crystal, micelle, liquid crystal, liposome, niosome, sols, sol gel, semisolid or macrosolid suspension, microencapsulated forms (e.g., alginate beads or agar gel beads, particles (e.g., macro, micro and/or nano scale particles and/or spheres (e.g., microspheres (e.g., albumin microspheres), and/or crystals and/or other form in which a boundary layer is formed to surround the sensitizer and/or other components of the sensitizer solution, and/or the like), or combinations thereof, that are well known to those skilled in the art.

### Method of Using

The sensitizer solution 12 is applied (i.e., delivered) to a treatment site in a single application and energy of an appropriate energy emission profile (i.e., energy type, and/or intensity, and/or frequency, and/or repetition rate) is applied (i.e., delivered) to the treatment site for a period of time. The energy is applied during and/or immediately after solution delivery and/or after the sensitizer solution has been in contact with the treatment site for a period of time (i.e., a contact period). The contact period is between 0 seconds (i.e., simultaneous application of the sensitizer solution and exposure to the activating energy emission profile) and about 48 hours. For example, the contact period is about 30 seconds, or about 1 minute, or about 2 minutes, or about 5 minutes, or about 10 minutes, or about 15 minutes, or about 30 minutes, or about 60 minutes, or about 2 hours, or about 4 hours, or about 8 hours, or about 12 hours, or about 16 hours, or about 24 hours, or about 36 hours, or about 48 hours. The sensitizer solution 12 is repeatedly applied, for example on a daily or multiple times per day basis, to the treatment site. The sensitizer solution 12 is applied directly to the treatment site and or to an applicator (e.g., mouthpiece, flexible applicator, bite block, toothbrush) that is then used to apply the solution to the treatment site.

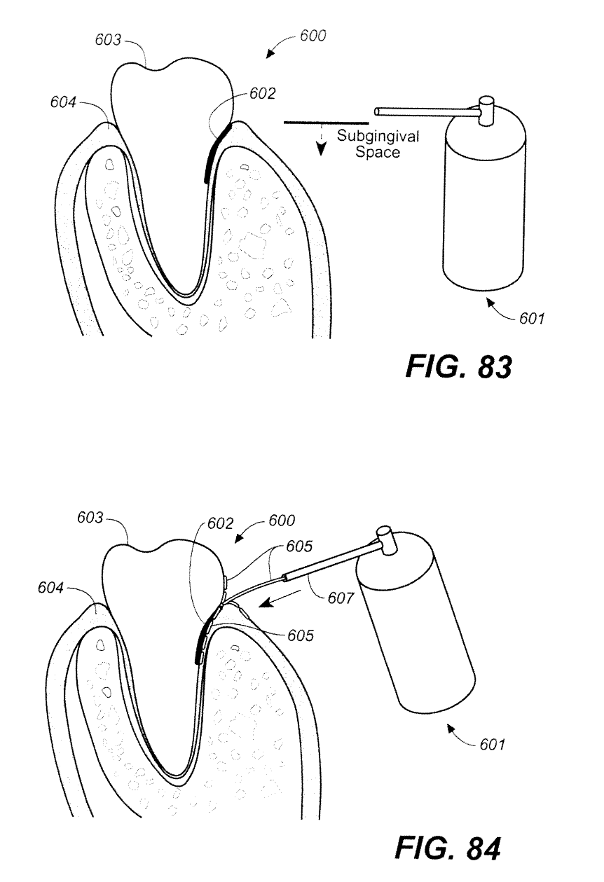

FIG. 83 illustrates a method of using the cleaning system 601 to clean a treatment site 600. The treatment site 600 includes an internal and/or external body surface or tissue, for example an oral cavity or surface, an organ surface such as the digestive tract (e.g., oral cavity, pharynx, esophagus, stomach, small intestine, large intestine, anus), urinary tract (e.g., renal pelvis, ureter, urethra, bladder), male reproductive tract (e.g., vas deferens, prostate, epididymis, testes), female reproductive tract (e.g., vagina, cervix, uterus, fallopian tubes, ovaries), respiratory tract (e.g., nose, sinus, pharynx, larynx, trachea, lungs), outer ear, auditory canal, middle ear, inner ear, eye (e.g., retina, vitreous, lens, cornea), circulatory system (e.g., blood vessel, arterial and/or venous, heart), lymphatic system, skin, arm pit, groin, mucosal surface, cerebrospinal system (e.g., brain, spinal chord, nerve fiber, cerebrospinal fluid, meninges, subarachnoid space), joints, bursa sack, bone marrow, cartilage, ligament, tendon, potential spaces (e.g., between layers of fascia), abdominal cavity, pericardial sac, and thoracic cavity. The treatment site includes a non-surface in the body, for example in the blood stream, cerebrospinal fluid, lymphatic fluid, in the body, or in a tissue such as in the gingiva, musculature, bone, teeth, or combinations thereof. The treatment site is an inanimate item, for example, floors and/or flooring materials (e.g., Linoleum, tile, carpet, wood, paint, etc.), walls and/or wall covering materials (e.g., paint, wall paper, Formica, tile, etc.) windows and/or window materials (glass, Plexiglas, polycarbonate, etc.), water delivery system components (e.g., pumps, pipes, reservoirs), structural and/or cosmetic building materials (e.g., concrete, masonry, stucco, steel, stainless steel, aluminum, copper, nickel, cast iron, plastic, fiberglass, carbon fiber, Kevlar) counters, furniture, clothes, dishes, or combinations thereof.

Cleaning includes the act of antisepsis, anti-necrosis, anti-inflammatory, removal of plaque, biofilm, and/or accretions, removal, and/or dilution, and/or inactivation of inflammatory agents, removal of extracellular material, other debris removal, destruction of biofilms, disinfecting, prophylaxis, antibiosis, mechanical removal or destruction, killing of biological organisms (e.g., microorganisms, insects), killing of cells (e.g., body cells, cancer cells, diseased cells), removal and/or bleaching of colored and/or discolored compounds and/or materials, or combinations thereof. The cleaning system is used to treat a patient suffering from a sepsis and/or cancer, for example by systemically administering a therapeutically effective amount of sensitizer solution to the patient wherein the sensitizer and/or a component therein has a high specificity for the targeted microorganisms and/or cancerous cells. After a delay, energy, for example light energy, is applied to a target site in and/or on the patient. The light has a wavelength that sufficiently penetrates target site and/or the patient and activates the administered sensitizer.

The cleaning system 601 is used to diagnose a patient with a sepsis or cancer. The sensitizer solution is delivered (e.g., oral, parenterally, including by injection, or topically) to the patient. The sensitizer has a high specificity for the targeted microorganisms and/or cancerous cells. After a time delay, the sensitizer is activated to emit a “wavelength” of light, for example by delivering an activating wavelength of light to a photosensitizer. The photosensitizer's emitted light is then detected.

The particular energy emission profile used during method of diagnosis is the same or different from the energy emission profile used to activate the toxic effects of the sensitizer. The transducers used to produce the diagnostic energy emission is in the cleaning system or separate from the cleaning system.

Application of the solutions and methods disclosed herein enable the prevention, treatment and/or diagnosis of a wide variety of diseases and/or conditions, for example diseases and or conditions caused and/or exacerbated by microorganisms (e.g., bacteria, including multiply-antibiotic resistant strains of bacteria, and/or viruses, fungi, protozoa), insects, and/or autologous cells (e.g., cancer, immune cells). The sensitizer solutions and methods disclosed herein are used in the prevention, treatment, and/or diagnosis of the following non-exclusive list of diseases and conditions and/or their symptoms in a patient: adenoma of the prostate gland, transplant rejections (e.g., using sensitizers to kill immune cells), benign prostatic hypertrophy, chronic prostatitis, otorhinolaryngologic diseases, (e.g., sinusitis, frontitis, polyposis), neovascular ophthalmic diseases (e.g., wet AMD, diabetic retinopathy, neovascular retinal diseases, central retinal vein occlusion, rubeosis iridis, herpes simplex, keratitis, trachoma, ptenygium histoplasmosis, subfoveal choroidal neovascularization), atherosclerotic plaques (e.g., photoangioplasty), canker sores, periodontitis, chronic and acute gingivitis (e.g., acute necrotizing ulcerative gingivitis, acute membranous gingivitis, ftisospirillary gingivitis, fusospirillosis, fusospirochetal gingivitis, necrotizing gingivitis, phagedenic gingivitis, ulcerative gingivitis, Vincent's gingivitis, Vincent's infection, Vincent's stomatitis), halitosis, tuberculosis, pneumonia, alveolitis, athletes foot, jock itch, ring worm, tape worms, candidiasis (e.g., oral candidiasis), mastitis, autoimmune diseases (e.g., using sensitizers to kill immune cells that can cause multiple sclerosis, rheumatoid arthritis), septicemia, bacterial infections, yeast infections, viral and inflammatory diseases, cervicitis, endometriosis, uterine fibroids, genital verucca, warts, pelvic inflammatory disease, Chlamydia disease, pre-malignant, carcinoma in situ of the cervix, acne, rosacea, psoriasis, herpes, papillomas, suppurative wounds, ulcers (e.g., of the skin, respiratory tract, digestive tract (e.g., oral, esophageal, stomach (for example those caused by the bacteria helicobacter pylori), intestine, rectum), herpes zoster, seborrheac dermatitis, leucoplakia, histoplasmosis, coccidiomycosis, hair removal, mole removal, keloid scars, tattoos, diseases of the joints (e.g., rheumatoid arthritis, osteomyelitis), hormone deficiency, mental depression, veterinary diseases (e.g., cancer, suppurative wounds, ulcers), viral infections (e.g., human immunodeficiency virus type I, herpes simplex virus type I/II, human cytomegalovirus, measles, simian virus, papilloma virus) and leukemia. The prevention of infection in people who are prone to infection due to an underlying condition (e.g., naturally occurring (e.g., genetic) immune system deficiency, diabetes, certain mitral valve disorders, A.I.D.S patients) and/or therapy (e.g., chemotherapy and/or radiation therapy, such as for the treatment of cancer, immunosuppressants therapy (e.g., organ transplant patients)), or for whom an infection could be a serious, for example life threatening, condition, (e.g., recent surgical patients, patients who have weakened or failing organs).

The sepsis 602, infection, other debris, microorganisms, or discolorations at a treatment site to be cleaned are on a tooth 603 and/or gingiva 604 and/or mucosal and/or epidermal surface, for example, the surface of the tooth 603 and/or gingiva 604 and/or nasal cavity and/or epidermal surface and/or in the subgingival space.

FIG. 84 illustrates that the cleaning system 601 delivers sensitizer solution 605, as shown by arrow, to the sepsis 602. The sensitizer solution 605 is delivered under pressure. The sensitizer solution is delivered in a continuous stream. The sensitizer solution stream has a delivery pressure as it exits the fluid outlet. The sensitizer solution stream delivery pressure is from about 0.1 psig to about 100 psig for example about 0.5 psig, or about 1 psig, or about 2 psig, or about 5 psig, or about 10 psig, or about 20 psig, or about 40 psig, or about 60 psig, or about 80 psig, or about 100 psig. The velocity of the sensitizer stream is between about 0.01 r/sec and 20 m/sec, for example, about 0.5 m/sec.

The sensitizer stream has a diameter at the exit of the delivery conduit 607, for example the applicator and/or the delivery conduit, from about 0.005 in (0.125 mm) to about 0.16 in (4 mm) for example about 0.06 in (1.5 mm) The sensitizer solution has a flow rate. The flow rate of the sensitizer solution is from about 0.5 ml/min to about 1000 ml/min, for example from about 1 ml/min, or about 5 ml/min, or about 10 ml/min, or about 20 ml/min, or about 40 ml/min, or about 80 ml/min, or about 200 ml/min, or about 400 ml/min, or about 600 ml/min, or about 800 ml/min. The sensitizer solution 605 is delivered to the subgingival space along the tooth 603 and/or gingiva 604 surfaces. The sensitizer solution 605 covers and/or penetrates into the sepsis 602. The photosensitizer solution 605 covers and/or penetrates tissue around the sepsis 602.

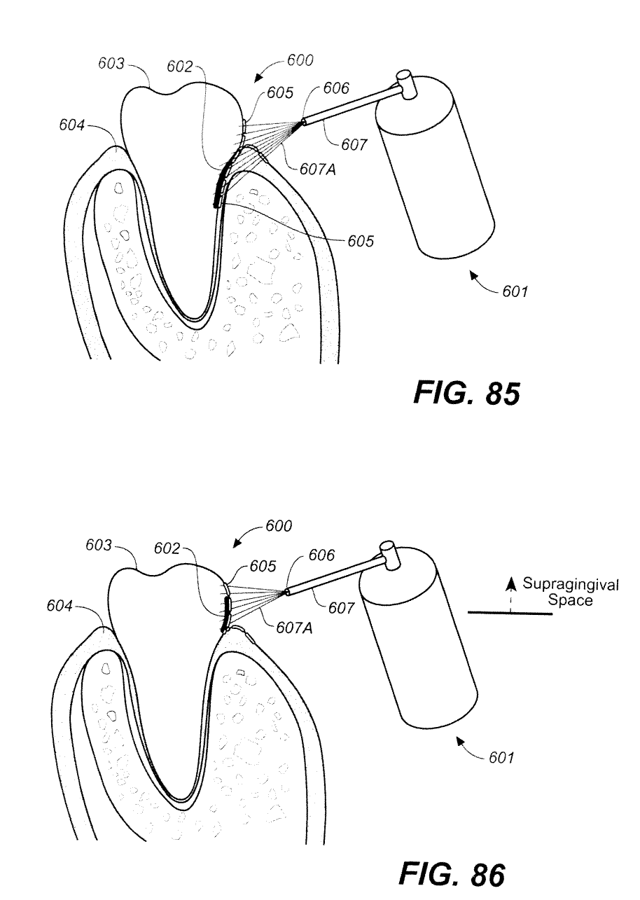

FIG. 85 illustrates that the illuminating device 606 emits the light energy 607A directly at a sepsis 602 that is located on, and/or within and/or below and/or behind a tissue structure. This location is invisible through direct line of site but fluidly connected to the external environment, for example in the subgingival space. The light energy 607A penetrates the tissues in the treatment site 600, for example the gingiva 604 and/or the tooth 603, and activate sensitizer solution 605 that is in locations, for example the subgingival space, that is difficult and/or impossible to visualize and/or access through other non-invasive and/or non-traumatic methods. Penetrating is defined as passing through a thickness of tissue while maintaining enough intensity to substantially activate the sensitizer solution. The light has a frequency between about 700 nm and about 1000 nm (e.g., far red, near-infrared, infrared).

The light energy 607A is absorbed by the sensitizer 605. The sensitizer is activated by the light energy 607A. The activated sensitizer reacts with free oxygen to produce singlet oxygen. Singlet oxygen has direct toxic effects on microorganisms and/or bleaching effects on colored compounds, and/or can undergo further non-photolytic reactions, for example chemical reactions, to produce other toxic reactive oxygen species (ROS), for example hydroxyl radical, superoxide anion, peroxides (e.g., (H₂O)), and hypochlorous acid (HOCl), which themselves have a toxic effect on microorganisms and/or bleaching effects on colored compounds.

FIG. 86 illustrates that the sepsis 602 is on a surface, for example the surface of a tooth 603, and/or the gingiva 604, and/or in a supragingival space, for example that formed by an ulcer or wound.

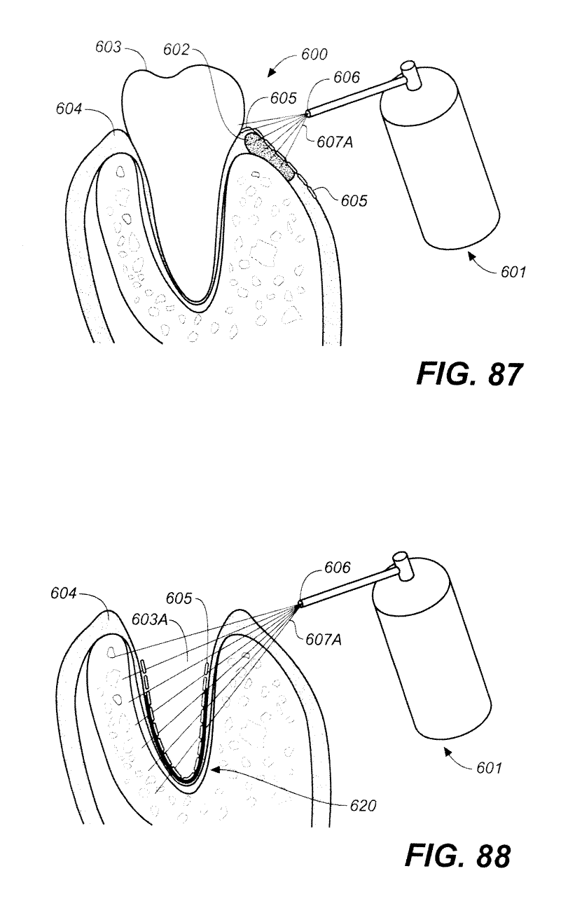

FIG. 87 illustrates that the sepsis 602 is in and/or on the gingiva 604 or tooth 603. The sensitizer solution 605 penetrates the tissue, and reaches sepsis 602 that is in the tissue. The sensitizer solution 605 penetrates into cracks, fissures, openings, pores, gaps, cavities, ulcerations, fistulas, abrasions, cuts and/or other openings, spaces, defects and/or breaks in the surface of the tooth and/or gingiva and/or mucosal and/or epidermal surface to reach any infective and/or inflammatory agents and/or inflammatory compounds that is located there. The sensitizer solution 605 is applied to any man made defect in the teeth and/or gingiva and/or mucosal and/or epidermal surface formed during, for example, dental and or medical procedures including surgical procedures (e.g., prophylaxis, caries treatment, amalgam installation and repair, root canal, veneer, inlay, onlay, crown, core buildup, pulp cap, pulpotomy, pulpal therapy, Endodontic procedures, apicoectomy/periraducular surgery, Periodontic procedures, gingivectomy, gingivoplasty, gingival flap procedure, surgical gingival curettage, osseous surgery, periodontal scaling and root plane, Prosthodontic procedures, tooth replacement, implant installation and/or maintenance and/or repair and/or replacement, alveoplasty, tooth extraction, surgical tooth extraction, orthodontia and dentofacial orthopedic installation and/or maintenance and/or repair and/or replacement, root extraction, removal of tumors and/or cysts and/or neoplasms, cosmetic and/or reconstructive and/or oral and maxillofacial surgery, intubation). The cleaning system is used to disinfect the treatment site 600, for example the oral cavity, in whole or in part before and/or during and/or after a dental and or medical procedure, including a surgical procedure. The cleaning system is used to prevent infection of the treatment site, for example the oral cavity, in whole or in part, by a microorganism. The cleaning system is used to treat the symptoms and/or underlying infection of a treatment site, for example the oral cavity, in whole or in part, by a microorganism.

FIG. 88 illustrates a method of cleaning a treatment site 620. The treatment site includes a wound or cavity, such as a wound or cavity formed by a trauma, or a dental and/or medical procedure, such as a tooth extraction 603A, root canal, removal of dental caries. The treatment site is in or on soft (e.g., gingival 604, skin, muscle) and/or hard (e.g., bone, tooth) tissue. The treatment site is in or on bone marrow. The cleaning system 601 delivers the sensitizer solution 605 to the treatment site 620. The cleaning system provides mechanical force, through the flow of the sensitizer solution, to aid in the penetration and distribution of the sensitizer solution as well as the removal of food debris, microorganisms, blood and/or blood components, necrotic tissue, biofilm, interstitial fluids, inflammatory compounds, and/or saliva. The cleaning system is used repeatedly to treat and/or prevent the infection of a treatment site 620, for example the system is used on a treatment site 620 after showering, or on a treatment site 620 in the oral cavity after eating and/or drinking. The cleaning system is used to accelerate the rate of healing of a treatment site 620, for example by preventing infection and/or inflammation (e.g., destroying and/or inhibiting microorganisms, removing and/or inhibiting inflammatory compounds) and/or by stimulating the natural healing responses (e.g., immune system, production of anti-inflammatory compounds) of the user. The cleaning system is used to increase the partial pressure of oxygen in the treatment site 620. The illumination energy of certain embodiments of the cleaning system also induces changes and/or reactions in the user that lead to healing, pain reduction, increased rate of cellular attachment to implants, and/or destruction of bacteria, cancer, or viruses, as is well known to those skilled in the art, for example those skilled in the use of light energy alone for therapeutic purposes, for example LLLT or LLLB.

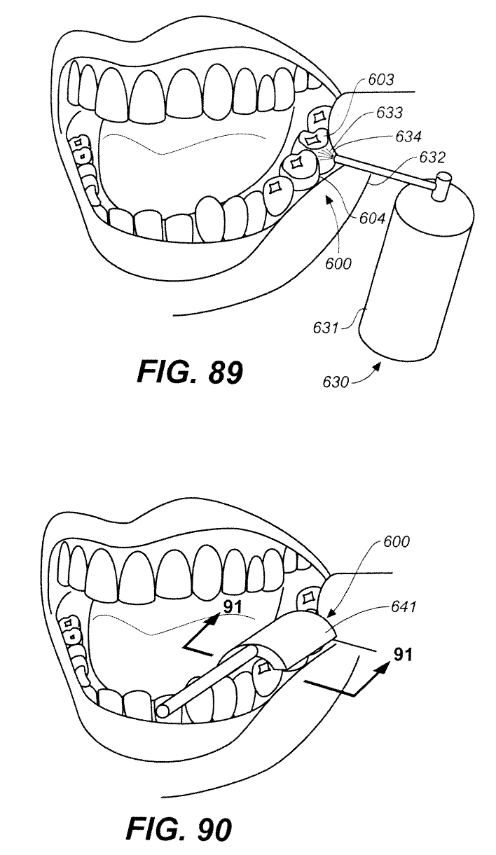

FIG. 89 illustrates a method of cleaning that includes the cleaning system 630 that has a fluid container 631. The delivery conduit 607 and/or fluid container 631 directly applies the sensitizer solution 633 to the desired sepsis, and/or tooth 603, and/or gingiva 604, and/or other oral surface. The illuminating device 634 emits the light energy 633 directly, and/or transgingivally, and/or transdentally to the sepsis and/or tooth 603, and/or gingiva 604, and/or other oral surface.

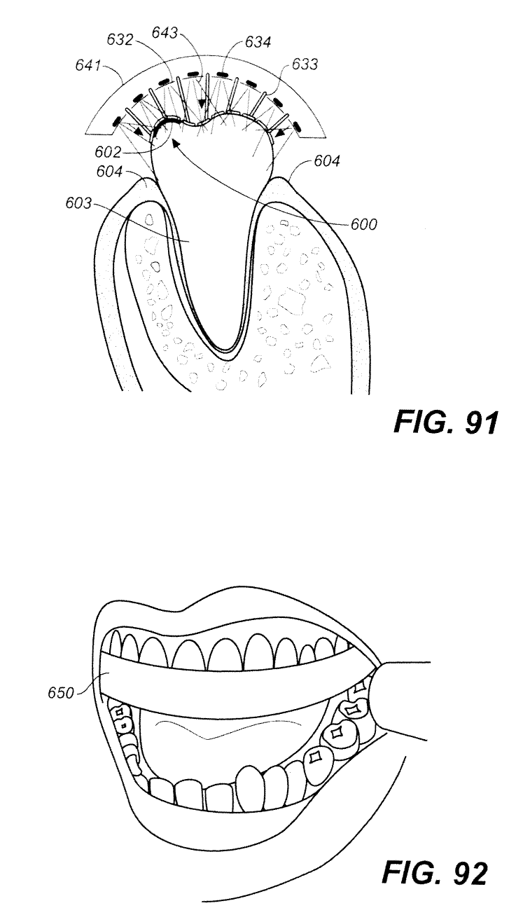

FIGS. 90 and 91 illustrate a method of cleaning that includes a cleaning system that has an applicator 641 (for illustrative purposes, the applicator 641 shown in FIG. 45). The applicator 641 is placed adjacent to the treatment site 600, for example a tooth 603, and/or gingiva 604, and/or other oral surface. The applicator 641 delivers the sensitizer solution 633 through the fluid outlets 632. The photosensitizer solution 633 is applied to the treatment site 600, for example manually and/or by a fluid delivery system, before the applicator 641 is placed adjacent to the treatment site 600. The applicator 641 emits the light energy 643 from the light sources 634.

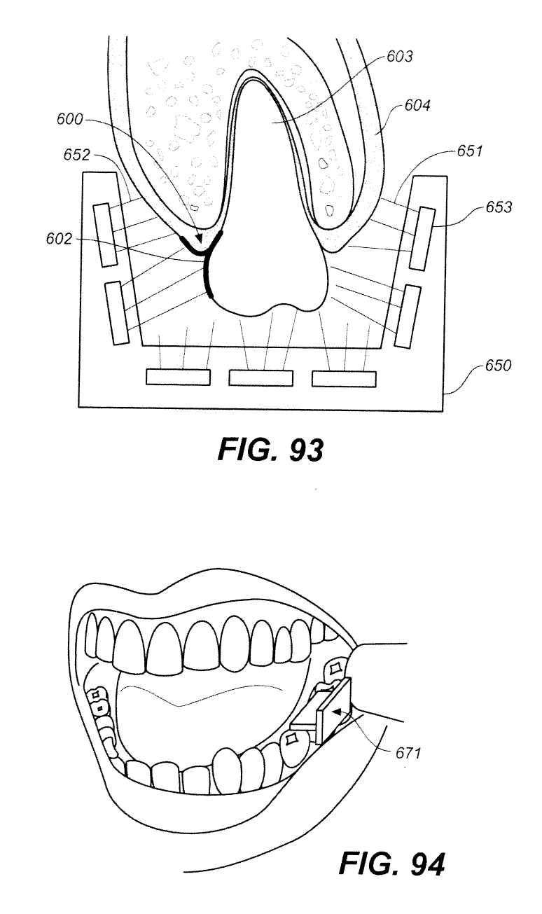

FIGS. 92 and 93 illustrate a method of cleaning that includes wearing a mouthpiece 650. The sensitizer solution can be delivered to the desired sepsis 602, and/or tooth 603, and/or gingiva 604, and/or other oral surface before the mouthpiece 650 is worn, and/or by applying the sensitizer solution to the mouthpiece before the mouthpiece is worn, and/or by eluting the sensitizer solution through the mouthpiece, and/or by delivering the sensitizer solution by another method described herein around and/or through the mouthpiece 650. The mouthpiece 650 has holes through which the sensitizer solution is delivered. The mouthpiece 650 is saturated and/or coated with sensitizer solution. The mouthpiece 650 emits light energy 651 and 652 from the illuminating devices 653 in and/or on the mouthpiece 650.

FIG. 94 illustrates a method of cleaning that includes inserting all or a portion of a mouthpiece, for example a bite block 671 in the mouth. The bite block 671 emits light energy from the illuminating devices in and/or on the bite block 671. Part or all of the bite block's 671 and the mouthpiece's sidewalls is in contact with or adjacent to the gingiva and/or one or more teeth, and or all or part of the hard and/or soft palate, and/or all or a part of the tongue, and/or all or a part of other oral surfaces (e.g., lips, mucosal surfaces).

The target organism, by way of example, is selected from a microorganism, e.g., a Bacteria, Archaea, Eukarya, virus, retrovirus, or bacteriaphage. The target organism is an insect. Further examples of Eukarya include, a fungal cell, a protozoan cell, a cell of Pneumocystis carinii, a parasitic helminth, or an arthropod. Further examples of Bacteria and Archaea include bacterial cells. Where the cell is a bacterial cell, the bacterial cell is a Gram positive or Gram negative bacterial cell, a Spirochete, Staphylococcus, Streptococcus, Enterococcus, Mycobacterium, Pseudomonas, Salmonella, Shigella, Escherichia, Erwinia, Klebsiella, Borrelia, Treponema, Campylobacter, Helicobacter, Bordetella, Neisseria, Legionella, Leptospira, Serpulina, Mycoplasma, Bacteroides, Klebsiella, Yersinia, Chlamydia, Vibrio, Actinobacillus, Porphyria, Hemophilus, Pasteurella, Peptostreptococcus, Listeria, Propionibacterium, Mycobacterium, Corynebacterium or Dermatophilus cell. The bacteria is one capable of living in the oral cavity, examples of which are Streptococcus mutans, Streptococcus sobrinus, Lactobacillus spp., Actinomyces spp., Bacteroides spp., Porphyromonas gingivalis, Prevotella intermedia, Actinobacillus actinomycetemcomitans, Fusobacterium nucleatum, Bacterioides forsythus, Streptococcus sanguis, Streptococcus mitis, Streptococcus oralis, Capnocytophaga spp., Wolinella recta, and Eikenella corrodens and combinations thereof.

Where the cell is a fungal cell, the cell is a Dermatophyte, a Candida or an Aspergillus cell.

The microorganism is Pneumocystis carinii.

Where the target organism is a protozoan cell, the cell is an Entamoeba, a Toxoplasma, a Giardia, a Leishmania, a Crytosporidium, or a Schistosoma.

Where the target organism is a virus, the virus is HIV, an HTLV, a hepatitis virus, an influenza virus, a rhinovirus, a papilloma virus, a measles virus, a Herpes virus, a rotavirus, a parvovirus, a psittacosis virus, Marburg virus, or an Ebola virus. The virus is a plant virus.

Where the target organism is an arthropod, the arthropod is a parasitic mite. Where the target organism is a helminth, the helminth is a nematode or a trematode. The arthropod is any member of the subphyla, classes, subclasses, orders, or species of the arthropoda phylum. (e.g., spiders, scorpions, centipedes, millipedes, insecta (e.g., cockroaches, termites, mantids, earwigs, flies, stoneflies, grasshoppers, locusts, walking sticks, plasmatodea, lice, thrips, mosquitoes, nats, mites, aphids, beetles, weevils)).

The terms target site and treatment site are synonymous and used interchangeably herein. The terms sidewall and sidewall panel are synonymous and used interchangeably herein.

### EXPERIMENTAL

### General

The following examples are provided to be illustrative and descriptive only. They are not to be construed to be limiting in any manner or fashion.

The components, sensitizers, solvents, oxygen sources, etc. are available from commercially available sources, e.g., see dental supply houses, and Chemical Sources USA published annually by Directories Research, Clemson, S.C., or Sigma/Aldrich Chemical Co., Milwaukee, Wis. The chemical components are used without purification unless otherwise noted.

### Example 1

### Teeth Whitener (ROS) Packaging System

(a) A commercial packaging system of FIG. 3 including an external delivery conduit and an internal delivery conduit of FIG. 4 is provided. The bladder of the bladder can, with polypropylene inner bladder lining, is filled to a volume of 80% with a commercially available hydrogen peroxide gel (e.g., Ultradent (about 9% hydrogen peroxide) and then pressurized with industrial grade oxygen to a pressure of 125 psi. A benefit in stability of the hydrogen peroxide based teeth whitening gel is realized. This benefit is increased as the concentration of peroxide in the gel is increased. The container can be sized for multiple uses.

The teeth whitening gel is dispensed as needed from the packaging system into a single use disposable applicator and applied to the dental arch desired to be whitened. Alternatively the gel is added to the channel(s) of a single or double sided illuminated applicator of FIG. 55 fitted with white LED based light sources. The teeth whitener loaded applicator is inserted into the users mouth and the light sources turned on (as indicated by an externally visible green LED) using the external control switch. A single activation of the control switch sets an internal timer for 10 minutes and sounds a single tone. A second activation of the control switch within 30 seconds of the first sets the internal timer for a total of 20 minutes sounds a double tone. When the cycle timer countdown reaches 10 minutes a single tone is generated. A third activation within 30 seconds of the second sets the internal timer for a total of 30 minutes and generates 3 tones. A tone is generated after each 10 minute period. When the timer reaches zero, from any set time, all LED's are turned off and a rapid series of 5 tones is generated. The applicator is rinsed and reused as desired.

A commercial packaging system of FIG. 7 with a cartridge constructed of polypropylene is filled with the commercially available hydrogen peroxide based gel (e.g., about 18% hydrogen peroxide) to a level of 90% and then pressurized to 125 psi using industrial grade oxygen gas. Professional application of this product is required. Before use of the product a dental professional protects the patients gingival tissues using techniques well known in the industry for such purposes. At the time of use the dental professional removes a protective seal from the valve of the cartridge and inserts the cartridge into the head of the packaging system engaging the cartridge valve. Force is then applied to the cartridge in the direction indicated in FIG. 7 to dispense the peroxide based gel.

The gel is dispensed into a single use disposable applicator (e.g., Flexible styrofoam tray) and inserted into the users mouth over the arch to be whitened.

The gel is dispensed into a cold sterilized re-usable applicator of FIG. 55 and used as indicated previously.
A) A lip/mouth retractor, well known for this purpose, is applied to the patients oral structures to prevent the soft tissues of the lips and cheeks from contacting the teeth desirous of whitening. The gel is dispensed directly onto the surfaces of the patients teeth desirous of whitening and allowed to remain there for a period of 30 minutes. The gel is cleaned from the teeth of the patient and the protecting devices removed.
B) Alternatively, after cleaning away the gel a second application of the gel is applied to the patients teeth desirous of whitening and the process repeated.
C) Alternatively, a commercially available external light source well known for the purposes of teeth whitening (e.g., BriteSmile) is used in conjunction with the whitening gel.
   (b) Similarly, the system of Example 1(a) includes a sensitizer, e.g., Toluidine Blue and optionally water.
   (c) Similarly, when Example 1 (b) is repeated except that the aqueous portion in the Toluidine Blue and any optional water is replaced with a stoichiometric or volumetric equivalent of oxidative reductive potential water solution as described in the incorporated by reference Oculus patent publications, then a corresponding whitening effect is observed.
   (d) Similarly, when Example 1 (a) or 1 (b) or 1 (c) is repeated except that the aqueous portion in Toluidine Blue and any option water is replaced with a stoichiometric or volumetric equivalent of oxidative reductive potential water solution as described in the incorporated by reference Oculus patent publications and a perfluorocarbon, e.g., perfluorodecalin is optionally present, then a corresponding whitening effect is observed.
   (e) Similarly, when Example 1 (a), (b), (c) or 1(d) is repeated except that the aqueous portion in Toluidine Blue and any optional water is replaced with a stoichiometric or volumetric equivalent of oxidative reductive potential water solution as described in the incorporated by reference Oculus patent publications and the oxygen pressure is 100 psi, then a corresponding whitening effect is observed.
   (f) Similarly, when Example 1 (b) is repeated except that the aqueous portion in Toluidine Blue and any optional water is replaced with a stoichiometric or volumetric equivalent of oxidative reductive potential water solution as described in the incorporated by reference Oculus patent publications and the photosensitizer and electromagnetic radiation treatment is not present, then a corresponding whitening effect is observed.
(g) Similarly, when Example 1 (a) is repeated except that any optional water is replaced with a stoichiometric or volumetric equivalent of oxidative reductive potential water solution as described in the incorporated by reference Oculus patent publications and the gel is optionally, in whole or in part, an emulsion as described in any of the incorporated by reference patents or patent publications of TherOx listed above, then a corresponding whitening effect is observed.
(h) Similarly, when Example 1 (b), (c), (d), (e), (f), is repeated except that the aqueous portion of Toluidine Blue and any optional water is replaced with a stoichiometric or volumetric equivalent of oxidative reductive potential water solution as described in the incorporated by reference Oculus patent publications and the gel is optionally, in whole or in part, an emulsion as described in any of the incorporated by reference patents or patent publications of TherOx listed above, then a corresponding whitening effect is observed.

### Example 2

### Wound Healing and Bacterial/Infection Control for Skin Ulcer

(a) A 68-year-old female is diagnosed with a long-term diabetic ulcer and infection about 2 in. in diameter just above the ankle. Previous multiple antibiotic treatment to shrink the infection have not been successful.

Treatment by use of this invention includes using a photosensitizing solution (e.g., as an emulsion comprising 15 micrograms/ml of Toluidine Blue O, 20% to 60% by wt. perfluorodecalin, and optionally other ingredients including water, and an emulsifying agent (e.g., lecithin) (total volume 400 cc) in a pressurized 500 cc bladder can of FIG. 3, including an internal delivery conduit of FIG. 4, at 125 psig with 100% oxygen.

After lavage of the treatment area via sterile saline, the oxygen enriched photosensitizing emulsion is sprayed directly onto the infected ulcer and the surrounding area (i.e., the treatment area), and allowed to remain in contact for a period of 2 to 30 minutes as decided by the practitioner (e.g., based on the level of tissue necrosis, patient history, patient comfort). An applicator similar to that in FIG. 48, fitted with commercial LED sources with a central wavelength of around 637 nm, is activated and positioned over the treatment area and allowed to remain for a period of 5 to 30 minutes. The applicator is then removed and the wound lavaged and covered in a generally accepted manner. This treatment significantly reduces the level of viable microorganisms, viruses and/or pathogens in the treatment area.

The patient is sent home with a treatment kit with instructions to read and understand the instruction manual and is then instructed to treat the infection twice a day as is described above. Within three days the patient reports that the infection is reduced and continues treatment. Observation by a profession after 7 days of treatment confirms that the infection area has been reduced by half and continued treatment as described results in the disappearance of the infection within one month.

(b) Similarly when Example 7(a) is repeated except that the 100% oxygen is replaced with a mixture of 80% oxygen and 20% ozone, then a corresponding reduction of the infection condition is observed.

(c) Similarly, when Example 2 (a) is repeated except that the aqueous portion in Toluidine Blue and any optional water is replaced with a stoichiometric or volumetric equivalent of oxidative reductive potential water solution as described in the incorporated by reference Oculus patent publications, then a corresponding reduction in the infection condition is observed.

(d) Similarly, when Example 2 (a) is repeated except that the aqueous portion in Toluidine Blue and any option water is replaced with a stoichiometric or volumetric equivalent of oxidative reductive potential water solution as described in the incorporated by reference Oculus patent publications and the perfluorodecalin is not present, then a corresponding reduction in the infection condition is observed.

(e) Similarly, when Example 2 (a) is repeated except that the aqueous portion in Toluidine Blue and any optional water is replaced with a stoichiometric or volumetric equivalent of oxidative reductive potential water solution as described in the incorporated by reference Oculus patent publications and the oxygen pressure is 100 psi, then a corresponding reduction in the infection condition is observed.

(f) Similarly, when Example 2 (a) is repeated except that the aqueous portion in Toluidine Blue and any optional water is replaced with a stoichiometric or volumetric equivalent of oxidative reductive potential water solution as described in the incorporated by reference Oculus patent publications and the photosensitizer and electromagnetic radiation treatment is not present, then a corresponding reduction in the infection condition is observed.

(g) Similarly, when Example 2 (a) is repeated except that the aqueous portion in Toluidine Blue and any optional water is replaced with a stoichiometric or volumetric equivalent of oxidative reductive potential water solution as described in the incorporated by reference Oculus patent publications and the gel is optionally, in whole or in part, an emulsion as described in any of the incorporated by reference patents or patent publications of TherOx listed above, then a corresponding reduction in the infection condition is observed.

(h) Similarly, when Example 2 (b), (c), (d) (e) or (f) is repeated except that the aqueous portion of Toluidine Blue and any optional water is replaced with a stoichiometric or volumetric equivalent of oxidative reductive potential water solution as described in the incorporated by reference Oculus patent publications and the gel is optionally, in whole or in part, an emulsion as described in any of the incorporated by reference patents or patent publications of TherOx listed above, then a corresponding reduction in the infection condition is observed.

### Example 3

### Prophylactic, Acute, Long-Term Oral Bacterial Control

(a) A 50-year-old man with a history of diabetes presents with gingival inflammation, sensitivity and recession. Routine examination results in a diagnosis of generalized class III periodontal disease, with measured subgingival pockets of up to 6 mm. The patient is prepared for scale and root plane on the left upper and lower dental quadrants according to standard practice. Based on a history of required AHA prophylactic antibiotics the patient is treated with the cleaning system prior to scale and root plane, in addition to after, which is the more normal course of therapy. The cleaning system, equipped with a pressurized canister of sensitizer solution (comprising 50 micrograms/ml Toluidine Blue O, perfluorodecalin, 10% to 60% by wt., and optionally other ingredients including water, emulsifier, RCS, e.g., oxygen and/or ozone, and flavorant(s)) is incorporated into the ultrasonic scaler, having separate water jet capability, used by the dental professional. Sensitizer solution is delivered as a fluid jet into the subgingival treatment area and the surrounding supragingival areas (e.g., 0.5 to 3 cc's per tooth with a suction tube used to remove excess sensitizer solution. The sensitizer solution is allowed to remain in contact with the treatment area for between 2 and 10 minutes as decided by the dental professional. An LED light wand, with a central wavelength of around 637 nm, similar to FIG. 44, optionally equipped with an optical fiber for delivery of light deep into a tooth sulcus, is used to activate the sensitizer in the deepest pockets, as decided by the dental professional, for a period of 30 seconds to 2 minutes. Additionally, or in place of the light wand, a whole mouth illuminator similar to that of FIG. 55 is used to activate the sensitizer. This treatment significantly reduces the level of viable microorganisms, viruses and/or pathogens in the treatment area.

The scale and root plane treatment is performed according to standard practice. Upon completion the cleaning therapy as described above can be repeated as decided by the dental professional (e.g., based on the specific needs of the patient).

The patient is given sample 50 ml pressurized canister of sensitizer solution (comprising 15 micrograms/ml Toluidine Blue O, perfluorodecalin, 10% to 60% by wt., and optionally other ingredients including water, emulsifier, RCS, e.g., oxygen and/or ozone, and flavorant(s)), a prescription for an applicator, similar to that of FIG. 26, and an appropriate amount of additional sensitizer, based on their needs as decided by the dental professional. They are given verbal and written instructions for the administration of the therapy in a non-professional environment. The patient returns for application of the therapy to the right side of their oral cavity as described above. Examination of the previously treated left side reveals acceptable recovery of the treated tissues. The patient returns after 3 months for follow up examination revealing reduced pain and sensitivity as well as average reduction of pocket depth by ⅓^{rd} to a maximum of 4 mm's. A prescription is given for additional sensitizer solution and the patient is instructed to continue therapy at home. At the 6 month recall appointment further stabilization and improvement are observed and the patient is reclassified as a class II periodontal patient with no pocket depths in excess of 4 mm.

(b) Similarly when example 3 (a) is repeated with the exception that no perfluorocarbon (perfluorodecalin) is present similar improvement is observed.

(c) Similarly, when Example 3 (a) is repeated except that the aqueous portion in Toluidine Blue and any optional water is replaced with a stoichiometric or volumetric equivalent of oxidative reductive potential water solution as described in the incorporated by reference Oculus patent publications, then a corresponding improvement is observed.

(d) Similarly, when Example 3 (a) is repeated except that the aqueous portion in Toluidine Blue and any option water is replaced with a stoichiometric or volumetric equivalent of oxidative reductive potential water solution as described in the incorporated by reference Oculus patent publications and the perfluorodecalin is not present, then a corresponding improvement is observed.

(e) Similarly, when Example 3 (a) is repeated except that the aqueous portion in Toluidine Blue and any optional water is replaced with a stoichiometric or volumetric equivalent of oxidative reductive potential water solution as described in the incorporated by reference Oculus patent publications and the oxygen pressure is 100 psi, then a corresponding is observed.

(f) Similarly, when Example 3 (a) is repeated except that the aqueous portion in Toluidine Blue and any optional water is replaced with a stoichiometric or volumetric equivalent of oxidative reductive potential water solution as described in the incorporated by reference Oculus patent publications and the photosensitizer and the electromagnetic radiation treatment is not present, then a corresponding improvement is observed.

(g) Similarly, when Example 3 (a) is repeated except that the aqueous portion in Toluidine Blue and any optional water is replaced with a stoichiometric or volumetric equivalent of oxidative reductive potential water solution as described in the incorporated by reference Oculus patent publications and the gel is optionally, in whole or in part, an emulsion as described in any of the incorporated by reference patents or patent publications of TherOx listed above, then a corresponding improvement is observed.

(h) Similarly, when Example 3 (a), (c), (d) (e) or (f) is repeated except that the aqueous portion of Toluidine Blue and any optional water is replaced with a stoichiometric or volumetric equivalent of oxidative reductive potential water solution as described in the incorporated by reference Oculus patent publications and the gel is optionally, in whole or in part, an emulsion as described in any of the incorporated by reference patents or patent publications of TherOx listed above, then a corresponding improvement is observed.

It is apparent to one skilled in the art that various changes and modifications are made to this disclosure, and equivalents employed, without departing from the spirit and scope of the invention. Elements shown with any embodiment are exemplary for the specific embodiment and are used on other embodiments within this disclosure. For example, a vibrating device, acoustic source, ultrasonic energy source, illuminating device, electromagnetic energy source (e.g., light source), electric energy source, magnetic energy source, and thermal energy source are substituted for each other throughout this disclosure. Also for example, fluid cartridges, cartridges, reservoirs, and fluid containers are substituted for each other throughout this disclosure. Also, for example the sensitizer solution is substituted with the sonosensitizer solution and/or the photosensitizer solution, and the sonosensitizer solution is substituted with the photosensitizer solution and vice versa, for example, along with substituting the acoustic transducer with the light source or illuminating device, and vice versa. Any species of transducer listed herein is substituted for any other species of transducer, for example along with substituting the appropriate species of sensitizer solution.

I claim:

1. A treatment system comprising:
a delivery system configured to dispense a treatment solution at a treatment site, the dispensed treatment solution comprising a carrier including a sensitizer, a chemical species, and a solvent within which the chemical species is soluble; and
an activation device configured to deliver energy to the treatment solution at the treatment site, where the delivered energy is within a range appropriate to cause the sensitizer to promote the formation of a reactive chemical species from the chemical species.

2. The treatment system of claim 1 wherein the delivery system comprises a pressurized container.

3. The treatment system of claim 2 wherein the pressurized container is pressurized with ozone.

4. The treatment system of claim 1 wherein the sensitizer comprises toluidine blue.

5. The treatment system of claim 1 wherein the sensitizer comprises a photosensitizer and the activation device includes a light source.

6. The treatment system of claim 5 wherein the light source is configured to emit visible light.

7. The treatment system of claim 5 wherein the light source is configured to emit IR light.

8. The treatment system of claim 1 wherein the sensitizer comprises a sonosensitizer and the activation device includes an acoustic energy source.

9. The treatment system of claim 1 wherein the chemical species comprises molecular oxygen and the reactive chemical species comprises singlet oxygen.

10. The treatment system of claim 1 wherein the delivery system stores the sensitizer and the chemical species separately and is configured to mix the sensitizer and the chemical species to form the treatment solution.

11. The treatment system of claim 10 wherein the delivery system is configured to mix the sensitizer and the chemical species to form the treatment solution at the treatment site.

12. The treatment system of claim 10 wherein the delivery system is configured to mix the sensitizer and the chemical species to form the treatment solution before dispensing the treatment solution at the treatment site.

13. The treatment system of claim 1 wherein the carrier comprises water.

14. The treatment system of claim 1 wherein the carrier comprises a gel.

15. The treatment system of claim 1 wherein the carrier comprises a cream.

16. The treatment system of claim 1 wherein the solvent comprises a perfluorocarbon.

17. The treatment system of claim 1 wherein the solvent comprises perfluorodecalin.

18. The treatment system of claim 1 wherein the delivery system comprises an applicator.

19. The treatment system of claim 1 wherein the activation device is integral with the delivery system.

20. A treatment method comprising:
dispensing at a treatment site a treatment solution comprising a sensitizer, a chemical species, and a solvent within which the chemical species is soluble; and
forming a reactive chemical species by delivering energy to the treatment solution at the treatment site, where the delivered energy is within a range appropriate to cause the sensitizer to promote the formation of the reactive chemical species from the chemical species.

21. The treatment method of claim 20 wherein dispensing the treatment solution includes forming the treatment solution by mixing the sensitizer and the chemical species.

22. The treatment method of claim 21 wherein the mixing is performed at the treatment site.

23. The treatment method of claim 20 wherein delivering energy to the treatment solution at the treatment site includes delivering visible light to the treatment site.

24. The treatment method of claim 20 wherein delivering energy to the treatment solution at the treatment site includes delivering IR light to the treatment site.

25. The treatment method of claim 20 wherein delivering energy to the treatment solution at the treatment site includes delivering acoustic energy to the treatment site.

26. The treatment method of claim 20 wherein the treatment site comprises a non-biological surface.

27. The treatment method of claim 20 wherein the treatment site comprises a surface within an oral cavity.

28. The treatment method of claim 20 wherein the treatment site comprises diseased tissue.

29. The treatment method of claim 20 wherein the sensitizer is coupled to a targeting moiety characterized by an affinity to target such that the reactive chemical species is formed in proximity to the target.

30. The treatment method of claim 29 wherein the target comprises a microorganism.

31. The treatment method of claim 29 wherein the target comprises diseased tissue.
